(19) United States
(12) Patent Application Publication
(10) Pub. No.: US 2024/0062906 A1
(43) Pub. Date: Feb. 22, 2024

(54) SYSTEMS AND METHODS FOR TEST DEVICE ANALYSIS

(71) Applicant: Regeneron Pharmaceuticals, Inc., Tarrytown, NY (US)

(72) Inventor: Matthew F. WIPPERMAN, Brooklyn, NY (US)

(73) Assignee: Regeneron Pharmaceuticals, Inc., Tarrytown, NY (US)

(21) Appl. No.: 18/448,318

(22) Filed: Aug. 11, 2023

Related U.S. Application Data
(60) Provisional application No. 63/371,159, filed on Aug. 11, 2022.

(51) Int. Cl. G16H 50/20; G16H 10/20; G16H 20/30

(52) U.S. Cl. CPC G16H 50/20; G16H 10/20; G16H 20/30

(57) ABSTRACT

Embodiments disclosed herein are directed to systems and methods for validating a test device using machine learning models generated based on a production device. The test device may be a simpler or more updated device in reference to a production device. Aspects of validating a model based on subsets of clinical data are also disclosed. Aspects of identifying features to determine individual signatures are also disclosed. Aspects of an example gait analysis device are also disclosed.

### CROSS-REFERENCED TO RELATED APPLICATIONS

This application claims the benefit of priority to U.S. Provisional Application No. 63/371,159, filed Aug. 11, 2022, which is incorporated by reference herein in its entirety.

### TECHNICAL FIELD

Embodiments disclosed herein are directed to systems and methods for validating a test device using machine learning models generated based on a production device. The test device may be a simpler or more updated device in reference to a production device. Aspects of validating a model based on subsets of clinical data are also disclosed. Aspects of identifying features to determine individual signatures are also disclosed. Aspects of an example gait analysis device are also disclosed.

### INTRODUCTION

Traditional analysis for detecting a condition (e.g., a medical condition) is often conducted using complex devices in clinical settings. Such traditional analysis often requires large devices, one or more medical professionals to assist with conducting a test, and/or requires an individual to visit a clinical site to perform the testing. Simplified devices may be used to substitute for such traditional analysis. However, such simplified devices need to be tested to confirm their capabilities and models need to be generated for such testing.

For example, gait assessment plays several roles in clinical practice and research for neurological and musculoskeletal diseases: diagnostic workup; guiding treatment selection and measuring response; assessment of gait and balance pathophysiology. Traditional gait is assessed in-clinic under the supervision of a physician, typically in a specialized gait lab with a force platform and/or motion tracking system. Gait labs use equipment that enables the creation of extensive models of human movement. Such equipment may include, but is not limited to, force plates to measure ground reaction force (GRF), camera-based video analysis to enable mapping of an individual skeletal architecture, and/or electromyography to measure muscle activation during movement. While applications of gait labs are diverse, in the context of a clinical trial setting for endpoint development, these detailed models of an individual's gait are likely not required, and stand-alone components of the gait lab such as force plates may provide sufficient disease-relevant information.

### SUMMARY OF THE DISCLOSURE

Aspects of the present disclosure relate to validating a test device using a trained machine learning model generated based on a production device. In one aspect, the present disclosure is directed to receiving sensed data from the production device for a control group, receiving sensed data from the production device for a target group having a target condition, training a machine learning model to identify a difference between the sensed data for the control group and the sensed data from the target group to generate the trained machine learning model, providing test sensed data from the test device for a test group comprising a plurality of individuals to the trained machine learning model, the plurality of individuals comprising first individuals having the target condition and second individuals not having the target condition, receiving a machine learning output from the trained machine learning model, the machine learning output categorizing the plurality of individuals as third individuals having the target condition or fourth individuals not having the target condition, comparing at least one of the first individuals to the third individuals or the second individuals to the fourth individuals to determine a match value, and validating the test device if the match value exceeds a match threshold.

Other aspects of the present disclosure relate to validating a test device using a machine learning model generated based on a production device. In one aspect, the present disclosure is directed to receiving a machine learning model trained to identify a difference between sensed data from the production device for a control group and sensed data from the production device for a target group, the target group having a target condition, providing test sensed data from the test device for a test group comprising a plurality of individuals to the trained machine learning model, the plurality of individuals comprising first individuals having the target condition and second individuals not having the target condition, receiving a machine learning output from the trained machine learning model, the machine learning output categorizing the plurality of individuals as third individuals having the target condition or fourth individuals not having the target condition, comparing at least one of the first individuals to the third individuals or the second individuals to the fourth individuals to determine a match value, and validating the test device if the match value exceeds a match threshold.

Other aspects of the present disclosure relate to validating a test device using a trained machine learning model generated using a production device. In one aspect, the present disclosure is directed to receiving sensed data from the production device for a control group, generating control analyzed data based on the sensed data from the production device for the control group, receiving sensed data from the production device for a target group having a target condition, generating target analyzed data based on the sensed data from the production device for the target group, training a machine learning model to identify a difference between the control analyzed data and the target analyzed data to generate the trained machine learning model, providing test sensed data from the test device for a test group comprising a plurality of individuals to the trained machine learning model, the plurality of individuals comprising first individuals having the target condition and second individuals as not having the target condition, receiving a machine learning output from the trained machine learning model, the machine learning output categorizing the plurality of individuals as third individuals having the target condition or fourth individuals not having the target condition, comparing at least one of the first individuals to the third individuals or the second individuals to the fourth individuals to determine a match value, and validating the test device if the match value exceeds a match threshold.

Other aspects of the present disclosure relate to validating a trained machine learning model. In one aspect, the present disclosure is directed to receiving sensed data for a first subset of individuals marked as being in a control group, receiving sensed data for a first subset of individuals marked as being in a target group having a target condition, training a machine learning model to identify a difference between the sensed data for the first subset of individuals marked as being in the control group and the sensed data for the first subset of individuals marked as being in the target group, to generate the trained machine learning model, providing unmarked test sensed data for a test group of individuals to the machine learning model, the test group of individuals comprising a second subset of individuals known to be in the control group and a second subset of individuals known to be in the target group, receiving a machine learning output from the trained machine learning model, the machine learning output categorizing each of the test group of individuals as being in the control group or being in the target group, comparing at least one of the test group of individuals categorized as being in the control group to the second subset of individuals known to be in the control group or the test group of individuals categorized as being in the target group to the second subset of individuals known to be in the target group to determine a match value, and validating the trained machine learning model if the match value exceeds a match threshold.

Other aspects of the present disclosure relate to validating a machine learning model. In one aspect, the present disclosure is directed to receiving a machine learning model trained to identify a difference between sensed data for a first subset of individuals marked as being in a control group and sensed data for a first subset of individuals marked as being in a target group, providing unmarked test sensed data for a test group of individuals to the machine learning model, the test group of individuals comprising a second subset of individuals known to be in the control group and a second subset of individuals known to be in the target group, receiving a machine learning output from the machine learning model, the machine learning output categorizing each of the test group of individuals as being in the control group or being in the target group, comparing at least one of the test group of individuals categorized as being in the control group to the second subset of individuals known to be in the control group or the test group of individuals categorized as being in the target group to the second subset of individuals known to be in the target group to determine a match value, and validating the machine learning model if the match value exceeds a match threshold.

Other aspects of the present disclosure relate to extracting features using a machine learning model. In one aspect, the present disclosure is directed to receiving sensed data for a first set of individuals, training a machine learning model to identify features that distinguish each individual in the first set of individuals from each other individual in the first set of individuals, to generate a trained machine learning model, and extracting the features from the trained machine learning model.

Other aspects of the present disclosure relate to characterizing unique individuals using a machine learning model. In one aspect, the present disclosure is directed to receiving sensed data for a first set of individuals, training a machine learning model to identify features that distinguish each individual in the first set of individuals from each other individual in the first set of individuals, to generate a trained machine learning model, receiving sensed data for a second set of individuals, providing the sensed data for the second set of individuals to the trained machine learning model, and receiving a machine learning output characterizing each individual of the second set of individuals as unique individuals based on the features.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1A is a system environment for validating a test device, in accordance with aspects of the present disclosure.

FIG. 1B is a flow chart for validating a test device, in accordance with aspects of the present disclosure.

FIG. 2A is a system block diagram for validating a machine learning model, in accordance with aspects of the present disclosure.

FIG. 2B is a flow chart for validating a machine learning model, in accordance with aspects of the present disclosure.

FIG. 3A is a flow chart for extracting features, in accordance with aspects of the present disclosure.

FIG. 3B is a flow chart characterizing individuals, in accordance with aspects of the present disclosure.

FIG. 4A shows force plate data sets, in accordance with aspects of the present disclosure.

FIG. 4B shows production device and test device data, in accordance with aspects of the present disclosure.

FIG. 4C also shows production device and test device data, in accordance with aspects of the present disclosure.

FIG. 4D shows two analytical methods, in accordance with aspects of the present disclosure.

FIG. 5A shows vertical ground reaction force (vGRF) curves from a production device and test device, in accordance with aspects of the present disclosure.

FIG. 5B shows a heat map based on vGRF curves, in accordance with aspects of the present disclosure.

FIG. 5C shows a Uniform Manifold Approximation and Projection (UMAP) dimensionality reduction, in accordance with aspects of the present disclosure.

FIG. 5D shows a schematic of a machine learning model, in accordance with aspects of the present disclosure.

FIG. 5E a receiver operating characteristic (ROC), in accordance with aspects of the present disclosure.

FIG. 5F shows a precision-recall curve for an XGBoost classification, in accordance with aspects of the present disclosure.

FIG. 6A shows a schematic of raw sensor time series data, according to an embodiment of the present disclosure.

FIG. 6B shows a correlation matrix of derived gait characteristics, according to an embodiment of the present disclosure.

FIG. 6C shows a heat map representation, according to an embodiment of the present disclosure.

FIG. 7A shows principal component analysis (PCA) dimensionality reduction of vGRF data, according to an embodiment of the present disclosure.

FIG. 7B shows a PCA dimensionality reduction of derived gait characteristics, according to an embodiment of the present disclosure.

FIG. 7C shows a PCA dimensionality reduction of raw sensor time series data, according to an embodiment of the present disclosure.

FIG. 7D shows ROC curves for target knee osteoarthritis (OA) versus control prediction, according to an embodiment of the present disclosure.

FIG. 7E shows precision-recall curves, according to an embodiment of the present disclosure.

FIG. 7F shows a heat map of OA verses control data at a comfortable speed, according to an embodiment of the present disclosure.

FIGS. 8A-8D show time series analyses of a test wearable insole device data, according to an embodiment of the present disclosure.

FIGS. 9A-9C show results based on training using raw sensor time series data from two days, according to an embodiment of the present disclosure.

FIG. 10 shows charts with data based on class, comparison metrics, methods, and comparisons, according to an embodiment of the present disclosure.

FIGS. 11A-11C show convolutional neural network (CNN) model results based on training across multiple days, according to an embodiment of the present disclosure.

FIG. 12 shows an overview of populations, according to an embodiment of the present disclosure.

FIGS. 13A-13F show vGRF data measured by production force plates and test wearable devices, according to an embodiment of the present disclosure.

FIG. 14 shows vGRF curves from control and knee injury populations, according to an embodiment of the present disclosure.

FIG. 15 shows z-scored vGRF curves on a per subject basis, according to an embodiment of the present disclosure.

FIG. 16 shows a UMAP generated using vGRF data, according to an embodiment of the present disclosure.

FIG. 17 shows a machine learning model, according to an embodiment of the present disclosure.

FIG. 18 shows predictive performance diagrams, according to an embodiment of the present disclosure.

FIG. 19 shows diagrams of an overview of types of data generated using a wearable device, according to an embodiment of the present disclosure.

FIG. 20 shows a heat map of distinct patterns, according to an embodiment of the present disclosure.

FIG. 21 shows a heat map showing correlations within and between categories of summary gait parameters, according to an embodiment of the present disclosure.

FIG. 22 show charts of vGRF data, of summary gait parameters, and of time series data, according to an embodiment of the present disclosure.

FIG. 23 shows charts of data points plotted based on available summary parameters and without walking speed included as a parameter, according to an embodiment of the present disclosure.

FIG. 24 shows charts showing that summary parameters outperform vGRF and time series data, according to an embodiment of the present disclosure.

FIG. 25 shows a chart and heat map of classification accuracy, according to an embodiment of the present disclosure.

FIG. 26 shows a heat map of intermediate activations extracted from a CNN model, according to an embodiment of the present disclosure.

FIG. 27 shows a CNN architecture, according to an embodiment of the present disclosure.

FIG. 28 shows CNN latent features, according to an embodiment of the present disclosure.

FIG. 29 shows analysis of latent CNN representations, according to an embodiment of the present disclosure.

FIG. 30 shows diagrams of CNN consistency models, according to an embodiment of the present disclosure.

FIG. 31 shows box charts based on training from one or two days, according to an embodiment of the present disclosure.

FIG. 32 shows UMAPs of latent features from a CNN model, according to an embodiment of the present disclosure.

FIG. 33 shows box charts of correlation data for participants, according to an embodiment of the present disclosure.

FIG. 34 shows a chart with correlation of CNN latent values, according to an embodiment of the present disclosure.

FIG. 35 shows another chart with correlation of CNN latent values, according to an embodiment of the present disclosure.

FIG. 36 includes a heat map of left and right foot GaitRec data, according to an embodiment of the present disclosure.

FIG. 37 shows charts and heat maps of right foot parameters, according to an embodiment of the present disclosure.

FIG. 38 shows data and analysis associated with a one or more speeds, according to an embodiment of the present disclosure.

FIG. 39 shows plots with WOMAC data and plots with Kellgren and Lawrence (KL) data, according to an embodiment of the present disclosure.

FIG. 40 shows plots of model predictions at comfortable speeds verses disease severity, according to an embodiment of the present disclosure.

FIG. 41 shows tables for per class binary classification metrics, auROC, auPR, and F1 scores for two datasets, according to an embodiment of the present disclosure.

FIG. 42 shows a diagram of a vGRF plot based on a gait cycle, according to an embodiment of the present disclosure.

FIG. 43 shows an example wearable insole device and sensor data, according to an embodiment of the present disclosure.

FIG. 44 shows sensor data for a wearable insole device, according to an embodiment of the present disclosure.

FIG. 45A-45D show wearable plate and wearable insole device based data, according to an embodiment of the present disclosure.

FIG. 46 shows a diagram of time series data, according to an embodiment of the present disclosure.

FIG. 47A-47F show vGRF force data measured by force plates and a wearable insole device, according to an embodiment of the present disclosure.

FIG. 48 shows a data flow for training a machine learning model, according to one or more embodiments.

FIG. 49 shows an example diagram of a computing device, according to one or more embodiments.

FIG. 50A shows a heatmap representation, according to an embodiment of the present disclosure.

FIG. 50B shows linear models, according to an embodiment of the present disclosure.

FIGS. 51A-51C show a schematic machine learning model and a ROC curve, according to an embodiment of the present disclosure.

FIGS. 52A-52E show PCA analyses and classification performances, according to an embodiment of the present disclosure.

FIG. 53 shows example heatmaps of walks and strides, according to an embodiment of the present disclosure.

FIG. 54 shows example boxplots of strides, according to an embodiment of the present disclosure.

FIG. 55 shows a chart of data points plotted based on max force and mean COP velocity, according to an embodiment of the present disclosure.

FIG. 56A shows examples boxplots of parameters including knee OA, control slow, comfortable, and fast walking speeds, according to an embodiment of the present disclosure.

FIG. 56B show charts of data points plotted based on (healthy control versus OA, according to an embodiment of the present disclosure.

FIGS. 57A-57B show example Spearman K-L correlations, according to an embodiment of the present disclosure.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

Aspects of the disclosed subject matter are directed to receiving signals (e.g., biometric signals) generated based on a body component of an individual. The signals may be or may be generated based on electrical activity, physical activity, biometric data, movement data, or any attribute of an individual's body, an action associated with the individual's body, reaction of the individual's body, or the like. The signals may be generated by a production device that may capture the signals using one or more sensors. For example, aspects of the disclosed subject matter are directed to methods for conducting gait assessment using a gait lab and/or force plates for generating, among other data, ground reaction force (GRF) data. As discussed herein, biosensor data collected by wearable devices (e.g., smart or digital insoles) may be comparable to lab-based clinical assessments and may be used to identify subject-specific gait patterns. In other examples, a lab-based gold standard may be used to identify subject-specific gait patterns.

Aspects of the disclosed subject matter are further directed to receiving signals generated using a test device. The signals generated using a test device may be similar to the signals generated using the production device, or may be signals generated to conduct analysis similar to analysis conducted using the production device. Analyzed data may be generated by applying a continuous function line based on sensed data and generating a stance phase based on the continuous function.

A production device may be one or more devices or systems that are known in a given industry as a gold standard device. As discussed herein, a gold standard device may be a device used to conduct a gold standard test. A gold standard test may be a diagnostic test or benchmark that is the best available under reasonable conditions. A gold standard device may be one that has been tested and has a reputation in the field as a reliable method. For example, for gait analysis a gold standard may include, but is not limited to, a gait lab including one or more force plates, sensors, cameras, or the like. A gait lab may use equipment that enables the creation of extensive models of human movement, including force plates to measure ground reaction force (GRF), video analysis to enable mapping of an individual skeletal architecture, and/or electromyography to measure muscle activation during movement.

A test device may be a non-gold standard device that may be used to generate results or analysis similar to a production device. A test device may be a simpler, newer, and/or unverified version of a production device. A test device may have a number of sensors. The number of sensors in or associated with the test device may be less than a corresponding production device. The sensors in or associated with the test device may be less dense than a corresponding production device. A test device may require validation to confirm that results provided by and/or analysis conducted using data output by the test device provides comparable performance (e.g., meets a threshold performance) to a production device. Test devices may be novel digital health technologies (DHTs) that require validation before being deployed. For example, for gait analysis, as discussed herein, a test device may be a wearable insole device that may be used to calculate vertical ground reaction forces (vGRF).

According to implementations of the disclosed subject matter a test device may be validated based on a machine learning model trained using a production device. For example, a wearable insole device may be validated based on a machine learning model trained using data generated at or related to a gait lab. As discussed herein, sensed data for a control group may be received from or generated at a production device. The sensed data may be output by one or more sensors associated with the production device. For example, the production device may correspond to a gait lab having one or more force sensor plates, cameras, etc. A user may use the gait lab and the force sensor plates, cameras, etc. may output sensed data.

The production device and test device may each be configured to output data that can be used to identify a given condition. The given condition may be a medical condition, a physical condition, or the like. For example, the given condition may be a disorder such as Parkinson's disease, progressive supranuclear palsy, multiple sclerosis, osteoarthritis (OA), or the like. The production device may be configured to sense data (e.g., vGRF data) that may be analyzed to determine whether a given individual has a given condition, based on the sensed data. The control group may include a group of individuals that are know not to have and/or exhibit the given condition.

Production sensed data for a target group with individuals having the given condition (e.g., a target condition) may be received from or generated at the production device. For example, the production device may first sense data for a control group of individuals. Accordingly, the production device may be used to generate or provided both sensed data for a control group and a target group, where the target group includes individuals having a given condition. Additionally, according to an implementation, production sensed data from a production device for the control group may be used to generate control analyzed data. Similarly, production sensed data from the production device for the target group may be used to generate target analyzed data. Production sensed data may be data detected by one or more sensors of the production device (e.g., a production system such as a gait lab). The one or more sensors may include, but are not limited to, pressure sensors, motion sensors, cameras, biometric sensors, environment sensors, weight sensors, accelerometers, gyroscopes, or the like. Additionally, according to an implementation, production sensed data from a production device for the target group may be used to generate target analyzed data.

A machine learning model may be trained to identify a difference between the sensed data for the control group and the sensed data for the target group. A trained machine learning model may be generated based on the training. Different techniques to train machine learning models are disclosed herein. For example, supervised machine learning may be used to train the machine learning model such that, during training, the sensed data for the control group is marked as such and sensed data for the target group is marked as such. Accordingly, the machine learning model may be trained to identify the differences between the sensed data for the control group verses the sensed data for the target group, based on the markings.

Test sensed data sensed using a test device may be generated. The test data may be sensed for a test group of individuals that includes both individuals without the given condition and users that have the given condition. For example, the test device may be different than the production device and may be used by a group of individuals to generate the test sensed data. Whether an individual in the test group has the given condition or does not have the given condition may be known, though the test sensed data may not be marked to indicate whether a given user has or does not have the condition.

The test sensed data may be provided to the trained machine learning model, trained using the production sensed data. The trained machine learning model may receive the test sensed data and may generate a machine learning output based on the test sensed data. The machine learning output may categorize each or a subset of individuals in the test group as either having the given condition or as not having the given condition. The machine learning output categorizations may be compared to the known categorization of each respective individual. The comparison may be a determination of whether the individuals categorized by the machine learning model as having the given condition are known to have the given condition and/or whether the individuals categorized by the machine learning model as not having the given condition are known to not have the given condition. A match value may be determined based on the comparison and may quantify or qualify the degree to which the machine learning model correctly categorizes the test group. The match value may be compared to a match threshold, and if the match value meets or exceeds the match threshold, then the test device may be validated. Validation may mean that the test device performs at least as well as the production device to categorize individuals, as dictated by the match threshold.

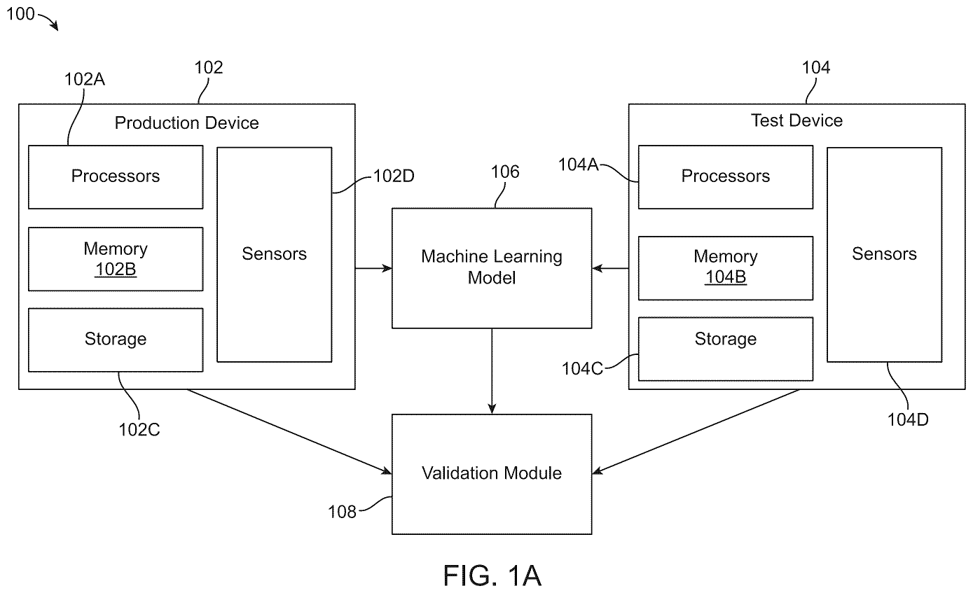

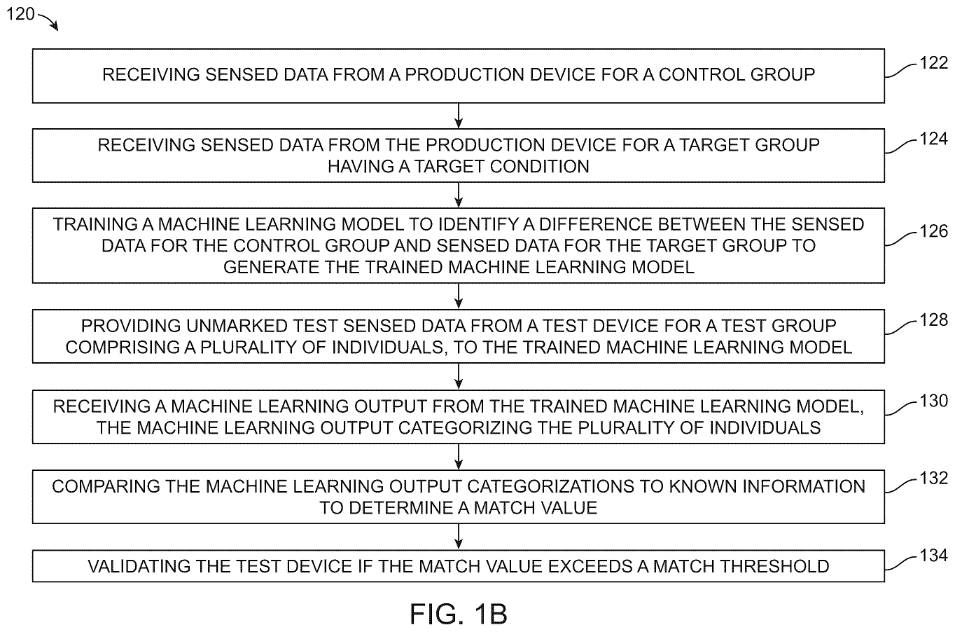

FIG. 1A shows a system environment 100 for validating a test device in accordance with the subject matter disclosed herein. As shown, a production device 102 may include one or more processors 102A, memories 102B, storage 102C, and/or sensors 102D. In some implementations, processors 102A may include one or more microprocessors, microchips, or application-specific integrated circuits. Memory 102B may include one or more types of random-access memory (RAM), read-only memory (ROM), and cache memory employed during execution of program instructions. Storage 102C may include one or more databases, cloud components, servers, or the like. Storage 102C may include a computer-readable, non-volatile hardware storage device that stores information and program instructions. Sensors 102D may be any sensors applicable to production device 102 and may include, but are not limited to, pressure sensors, motion sensors, cameras, biometric sensors, environment sensors, weight sensors, accelerometers, gyroscopes, or the like. Processors 102A may use data buses to communicate with memory 102B, storage 102C, and/or sensors 102D.

As also shown, a test device 104 may include one or more processors 104A, memories 104B, storage 104C, and/or sensors 104D. In some implementations, processors 104A may include one or more microprocessors, microchips, or application-specific integrated circuits. Memory 104B may include one or more types of random-access memory (RAM), read-only memory (ROM), and cache memory employed during execution of program instructions. Storage 104C may include one or more databases, cloud components, servers, or the like. Storage 104C may include a computer-readable, non-volatile hardware storage device that stores information and program instructions. Sensors 104D may be any sensors applicable to production device 102 and may include, but are not limited to, pressure sensors, motion sensors, cameras, biometric sensors, environment sensors, weight sensors, accelerometers, gyroscopes, or the like. Processors 104A may use data buses to communicate with memory 104B, storage 104C, and/or sensors 104D.

As shown in system environment 100, production device 102 and/or test device 104 may communicate with a machine learning model 106. Machine learning model 106 may be a standalone component or may be a part of production device 102 and/or test device 104. For example, production device 102 and/or test device 104 may communicate with machine learning model 106 over a network such that machine learning model 106 is a cloud component or stored at a cloud component. Machine learning model 106 may be implemented using one or more processors, memory, storage, or the like. According to an implementation, machine learning model 106 may receive data generated using sensors 102D and/or sensors 104D. Machine learning model 106 may receive the data directly from production device 102 and/or test device 104 (e.g., over a network) or may receive the data through a different component that receives the data from production device 102 and/or test device 104.

Validation module 108 may communicate with machine learning model 106, production device 102, and/or test device 104. Validation module 108 may receive a machine learning output (e.g., categorizations) from machine learning model 106 and may compare the output to known information (e.g., from production device 102 and/or test device 104). Validation module 108 may generate a match value based on the comparison and may compare the match value against a match threshold to validate test device 104.

FIG. 1B shows a flowchart 120 for validating a test device, in accordance with the subject matter disclosed herein. At step 122, sensed data from a production device for a control group may be received. The sensed data may be generated at one or more sensors 102D that may be part of a device or a system. The sensed data may be provided by processors 102A and may be stored at memory 102B and/or storage 102C such that processors 102A may retrieve the sensed data from memory 102B and/or storage 102C. The sensed data may be in the format output by one or more sensors 102D or may be in a different format. For example, processors 102A may receive the sensed data in a first format and may convert the sensed data to a second format. As further discussed herein processors 102A and/or one or more other components may generate analyzed data based on the sensed data. The control group may include individuals that are known not to have a given condition, as disclosed herein.

At step 124, sensed data from the production device for a target group may be received. The sensed data may be generated, provided, and/or formatted as disclosed in reference to the sensed data at step 122. The target group may include individuals that are known to have the given condition, as disclosed herein.

At step 126, a machine learning model may be trained to identify one or more differences between the sensed data for the control group (step 122) and the sensed data for the target group (step 124). A trained machine learning model may be generated based on the training. Training the machine learning model may include modifying one or more weights, biases, layers, nodes, or the like of the machine learning model based on the sensed data and/or differences in the sensed data for the control group and target group. Accordingly, the trained machine learning model may be configured to receive new sensed data (e.g., test sensed data as further discussed herein) to categorize an individual, to whom the new sensed data corresponds to, as either having the given condition or as not having the given condition. Techniques for training the machine learning model are further disclosed herein.

At step 128, unmarked test data from a test device for a test group may be provided to the trained machine learning model. The test group may include some individuals known to have the given condition and some individuals known not to have the given condition. The test sensed data may be unmarked such that the unmarked test data input to the machine learning model may not include a marker or other indication of whether a given individual who is part of the test group has or does not have the given condition. For example, the test group may include first individuals known to have the target condition and second individuals known to not have the target condition. Accordingly, the trained machine learning model may not receive a marker or other indication about whether each individual in the test group has or does not have the given condition. The unmarked test data may be processed by the trained machine learning model based on one or more of the weights, biases, layers, nodes, or the like of the trained machine learning model.

At step 130, a machine learning output may be received from the trained machine learning model. The machine learning model may categorize each of the plurality of individuals in the test group as respectively either having the given condition or not having the given condition. Accordingly, the trained machine learning model may independently determine whether a given individual is categorized as having the given condition or as not having the given condition, without prior input or knowledge of the same. For example, the machine learning output may categorize some of the individuals in the input test group as third individuals having the given condition or fourth individuals not having the given condition. It will be understood that some individuals from the test group may not be categorized as having the given condition or not having the given condition. For example, the unmarked test data for a given individual may be ambiguous such that the trained machine learning model may not categorize that given individual with a required level of certainty.

At step 132, the machine learning output categorizations may be compared to the known information about each individual in the test group, to determine a match value. For example, the first individuals (known to have the given condition) may be compared to the third individuals categorized by the machine learning output as having the given condition. Alternatively, or in addition, second individuals (known to not have the given condition) may be compared to the fourth individuals categorized by the machine learning output as not having the given condition. Accordingly, at step 132, a comparison of the known information for each respective individual may be compared to the categorization determined by the machine learning model.

A match value may be determine based on the comparison at step 132. The match value may be a quantitative or qualitative comparison and may indicate the degree to which the machine learning output correctly categorized individuals in the test group as either having or not having the given condition. The match value may be a numerical score, a correlation value, an overlap value, a percentage, a tier, or the like that indicates the success of the machine learning output in correctly categorizing the individuals in the test group as having or not having the given condition.

At step 134, a validation component (e.g., validation module 208) and/or the test device may determine if the match value meets or exceeds a match threshold. If the match value meets or exceeds the match threshold, then the test device may be validated. A match threshold format may be comparable to the match value format such that the match value can be compared to the match threshold. The match threshold may be predetermined, may be set (e.g., via user input), or may be dynamically determined. A dynamically determined match threshold may be dynamically determined using a match threshold machine learning model and/or an algorithm or other computation mechanism. A dynamic match threshold may be determined based on the given condition, the production device, the test device, the test group, or the like.

Accordingly, a validated test device may be a device that can be used to categorize individuals as having a given condition or not having a given condition, as tested against a publication device. A validated test device may be approved for use to determine presence of the given condition in a manner similar to determining the presence of the given condition using the production device. However, it will be understood that the test device may use different components (e.g., sensors) than the production device. For example, the test device may include simpler or different components than the production device, yet may be validated to perform the same test(s) as the production device.

The production device and/or the test device may each generate sensed data based on respective components (e.g., sensors). Accordingly, the sensed data from the production device may be in a different format, may be calibrated differently, may be categorized and/or stored differently, or the like, than the sensed device from the test device. For example, sensed data from a gait lab may include force plate data for number of sensors in one or more force plates at the gait lab and may also include camera data, motion data, etc. Sensed data from a wearable insole device may be pressure data detected by sensors contained within the insole. Accordingly, the sensed data from a production device may not provide a one-to-one comparison to sensed data from a test device. As a result, a machine learning model trained using production sensed data may not be configured to provide an applicable machine learning output based on test sensed data.

According to implementations of the disclosed subject matter, control analyzed data may be generated based on the control group production sensed data and target analyzed data may be generated based on the target group production sensed data. Similarly, test analyzed data may be generated based on the test sensed data. For example, production sensed data from a gait lab may be used to determine a control vGRF for each individual in the control group. Production sensed data from the gait lab may be used to determine a target vGRF for each individual in the target group. Similarly, test sensed data from the wearable insole device may be used to determine a test vGRF for each individual in the test group. Accordingly, each of the control analyzed data, the target analyzed data, and the test analyzed data may have a one-to-one correlation such that although the underlying sensed data may be incomparable for each of the control, target, and test groups, the analyzed data may be comparable.

The machine learning model may be trained based on the control analyzed data and the target analyzed data. Subsequently, test analyzed data may be provided to the trained machine learning model and a machine learning output may be generated based on the test analyzed data. In this implementation, the machine learning model may be trained using the same format or type of data as the machine learning model uses to generate a machine learning output. The machine learning output may categorize each or a subset of individuals in the test group as either having the given condition or as not having the given condition, based on their respective test analyzed data (e.g., test vGRF plots). The machine learning output categorizations may be compared to the known categorization of each respective individual in the test group. The comparison may be a determination of whether the individuals categorized by the machine learning model as having the given condition are known to have the given condition and/or whether the individuals categorized by the machine learning model as not having the given condition are known to not have the given condition. A match value may be determined based on the comparison and may quantify or qualify the degree to which the machine learning model correctly categorizes the test group. The match value may be compared to a match threshold, and if the match value meets or exceeds the match threshold, then the test device may be validated. Validation may mean that the test device performs at least as well as the production device to categorize individuals, as dictated by the match threshold.

According to an implementation of the disclosed subject matter, a trained machine learning model may be validated based a control group. Production sensed data for a first subset of a control group with individuals not having a given condition (e.g., a target condition) may be received from or generated at the production device. Similarly, production sensed data for a first subset of a target group with individuals having the given condition may be received from or generated at the production device. Accordingly, the production device may be used to generate or provided both sensed data for a first subset of the control group and a first subset of the target group, where the target group includes individuals having a given condition. Production sensed data may be data detected by one or more sensors of the production device (e.g., a production system such as a gait lab).

A machine learning model may be trained to identify a difference between the sensed data for the first subset of the control group and the sensed data for the first subset of the target group. A trained machine learning model may be generated based on the training. Different techniques to train machine learning models are disclosed herein. For example, supervised machine learning may be used to train the machine learning model such that, during training, the sensed data for the first subset of the control group is marked as such and sensed data for the first subset of the target group is marked as such. Accordingly, the machine learning model may be trained to identify the differences between the sensed data for the first subset of the control group verses the sensed data for the first subset of the target group, based on the markings.

A verification group may include a second subset of the control group with individuals known to not have the given condition and also a second subset of the target group with individuals known to not have the given condition. Production sensed data for the second subset of the control group with individuals known to not have the given condition may be received from or generated at the production device. Similarly, production sensed data for the second subset of the target group with individuals known to have the given condition may be received from or generated at the production device. The sensed data for the second subset of the control group and the second subset of the target group may not be marked. Unmarked verification sensed data may correspond to the sensed data for the second subset of the control group and the second subset of the target group (the verification group).

The unmarked verification sensed data for the verification group may be provided to the trained machine learning model. The trained machine learning model may receive the unmarked verification sensed data and may generate a machine learning output based on the same. The machine learning output may categorize each or a subset of individuals in the unmarked verification sensed data as either having the given condition or as not having the given condition. The machine learning output categorizations may be compared to the known categorization of each respective individual. The comparison may be a determination of whether the individuals categorized by the trained machine learning model as having the given condition are known to have the given condition (i.e., are part of the second subset of the control group) and/or whether the individuals categorized by the machine learning model as not having the given condition are known to not have the given condition (i.e., are part of the second subset of the target group). A match value may be determined based on the comparison and may quantify or qualify the degree to which the trained machine learning model correctly categorizes the test group. The match value may be compared to a match threshold, and if the match value meets or exceeds the match threshold, then the trained machine learning model may be validated. Validation may mean that the trained machine learning model is configured to categorize individuals as having or not having the given condition with a level of certainty, as dictated by the match threshold.

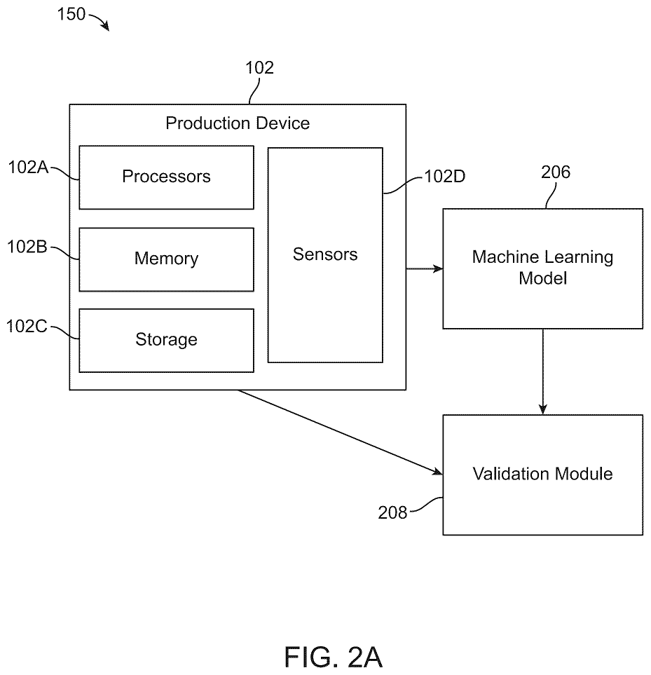

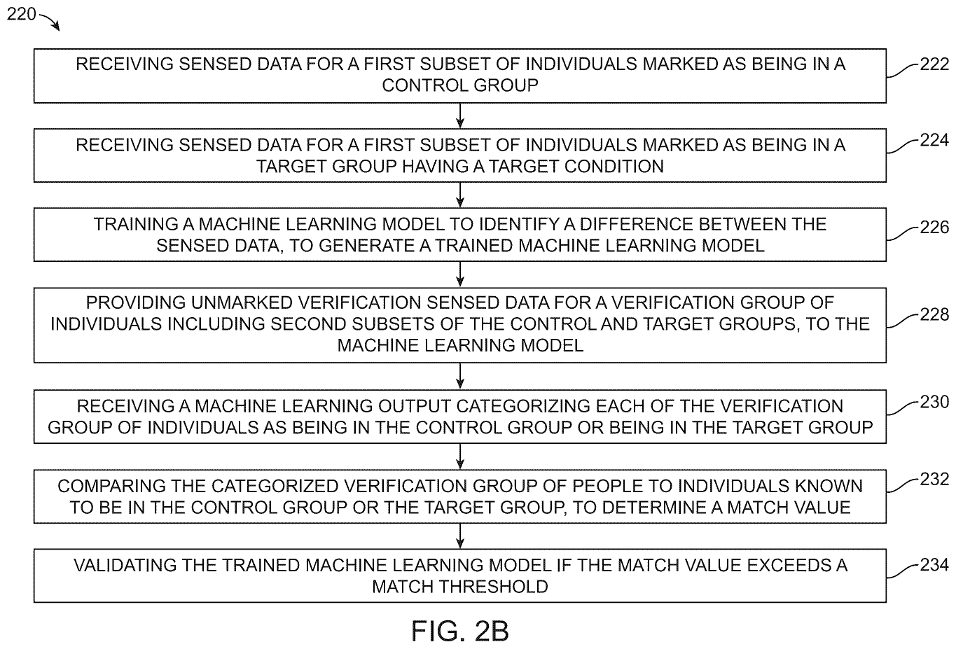

FIG. 2A shows a system environment 150 for validating a machine learning model in accordance with the subject matter disclosed herein. As shown, system environment 150 may include some components that are the same as or similar to the components of system environment 100 of FIG. 1A. Accordingly, such components are not described again for brevity. As shown, system environment 150 may include production device 102, processors 102A, memory 102B, storage 102C, and sensors 102D. Production device 102 and/or one or more of its components may communicate with machine learning model 206 which may be similar to or different than machine learning model 106 of FIG. 1A. Machine learning model 206 is further discussed herein.

Validation module 208 may communicate with machine learning model 206 and/or production device 102. Validation module 208 may receive a machine learning output (e.g., categorizations) from machine learning model 206 and may compare the output to known information (e.g., from production device 102). Validation module 208 may generate a match value based on the comparison and may compare the match value against a match threshold to validate test device 104.

FIG. 2B shows a flowchart 220 for validating a machine learning, in accordance with the subject matter disclosed herein. At step 222 sensed data from a production device for a first set of individuals marked as being in a control group may be received. The sensed data may be generated at one or more sensors 102D that may be part of a device or a system. The sensed data may be provided by processors 102A and may be stored at memory 102B and/or storage 102C such that processors 102A may retrieve the sensed data from memory 102B and/or storage 102C. The sensed data may be in the format output by one or more sensors 102D or may be in a different format. For example, processors 102A may receive the sensed data in a first format and may convert the sensed data to a second format. As further discussed herein processors 102A and/or one or more other components may generate analyzed data based on the sensed data. The control group may include individuals that are known not to have a given condition, as disclosed herein.

At step 224, sensed data from the production device for a first set of individuals marked as being in a target group may be received in a manner similar to that discussed for step 222. The target group may include individuals that are known to have the given condition, as disclosed herein.

At step 226, a machine learning model may be trained to identify one or more differences between the sensed data for the first subset of the control group (step 222) and the sensed data for the first subset of the target group (step 224). A trained machine learning model may be generated based on the training. Training the machine learning model may include modifying one or more weights, biases, layers, nodes, or the like of the machine learning model based on the sensed data and/or differences in the sensed data for the first subset of the control group and the first subset of the target group. Accordingly, the trained machine learning model may be configured to receive verification sensed data (e.g., sensed data for a verification group having second subsets of the control group and/or the target group, as further discussed herein) to categorize an individual, to whom the verification sensed data corresponds to, as either having the given condition or as not having the given condition. Techniques for training the machine learning model are further disclosed herein.

At step 228, unmarked verification sensed data from the production device for a verification group may be provided to the trained machine learning model. The verification group may include a second subset of the control group not having the given condition and a second subset of the target group having the given condition. The verification sensed data may be unmarked such that the unmarked verification sensed data input to the machine learning model may not include a marker or other indication of whether a given individual who is part of the verification group has or does not have the given condition. Accordingly, the trained machine learning model may not receive a marker or other indication about whether each individual in the verification group has or does not have the given condition. The unmarked verification sensed data may be processed by the trained machine learning model based on one or more of the weights, biases, layers, nodes, or the like of the trained machine learning model.

At step 230, a machine learning output may be received from the trained machine learning model. The machine learning model may categorize each of the plurality of individuals in the verification group as respectively either having the given condition or not having the given condition. Accordingly, the trained machine learning model may independently determine whether a given individual in the verification group is categorized as having the given condition or as not having the given condition, without prior input or knowledge of the same. It will be understood that some individuals from the verification group may not be categorized as having the given condition or not having the given condition. For example, the unmarked verification data for a given individual may be ambiguous such that the trained machine learning model may not categorize that given individual with a required level of certainty.

At step 232, the machine learning output categorizations may be compared to the known information about each individual in the verification group, to determine a match value. For example, the individuals in the second subset of the control group (known to have the given condition) may be compared to the individuals categorized by the machine learning output as having the given condition. Alternatively, or in addition, individuals in the second subset of the target group (known to not have the given condition) may be compared to the individuals categorized by the machine learning output as not having the given condition. Accordingly, at step 232, a comparison of the known information for each respective individual may be compared to the categorization determined by the machine learning model.

A match value may be determine based on the comparison at step 232. The match value may be a quantitative or qualitative comparison and may indicate the degree to which the machine learning output correctly categorized individuals in the verification group as either having or not having the given condition. The match value may be a numerical score, a correlation value, an overlap value, a percentage, a tier, or the like that indicates the success of the machine learning output in correctly categorizing the individuals in the verification group as having or not having the given condition.

At step 234, a validation component (e.g., validation module 208) may determine if the match value meets or exceeds a match threshold. If the match value meets or exceeds the match threshold, then the machine learning model trained at step 226 may be validated. A match threshold format may be comparable to the match value format such that the match value can be compared to the match threshold. The match threshold may be predetermined, may be set (e.g., via user input), or may be dynamically determined. A dynamically determined match threshold may be dynamically determined using a match threshold machine learning model and/or an algorithm or other computation mechanism. A dynamic match threshold may be determined based on the given condition, the production device, the test device, the test group, or the like.

Accordingly, a validated machine learning model may be a model that can be used to categorize individuals as having a given condition or not having a given condition, as tested against subsets of control and target individuals. A validated machine learning model may be approved for use to, for example, determine if a test device (e.g., as described in FIGS. 1A and 1B) is validated. For example, an untrained or previously trained version of a validated machine learning model may be trained at step 126 of FIG. 1B.

In a manner similar to that described above, the machine learning model may be trained based on control analyzed data and target analyzed data. The trained machine learning model may receive verification analyzed data and may generate a machine learning output based on the verification analyzed data.

According to an implementation of the disclosed subject matter, sensed data may be analyzed to determine if one or more features of the sensed data can be used to identify unique individuals. The one or more features may be used to generate a signature for a given signature such that the signature may be unique to that individual when compared to one or more other individuals.

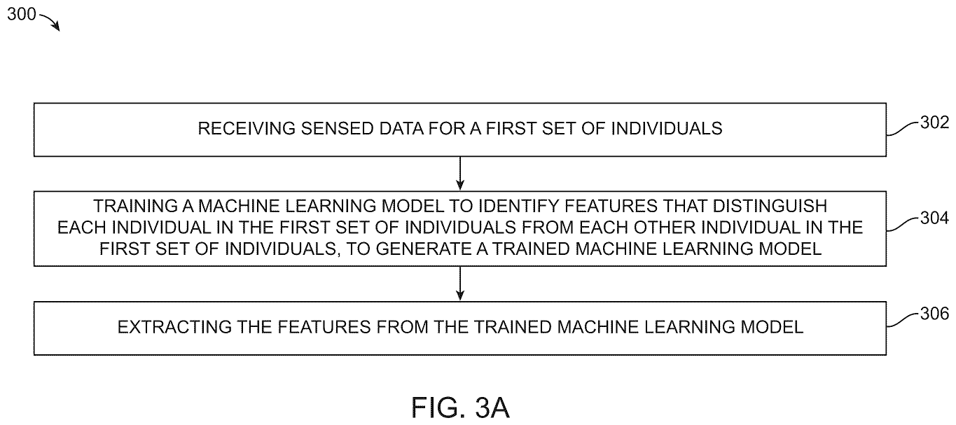

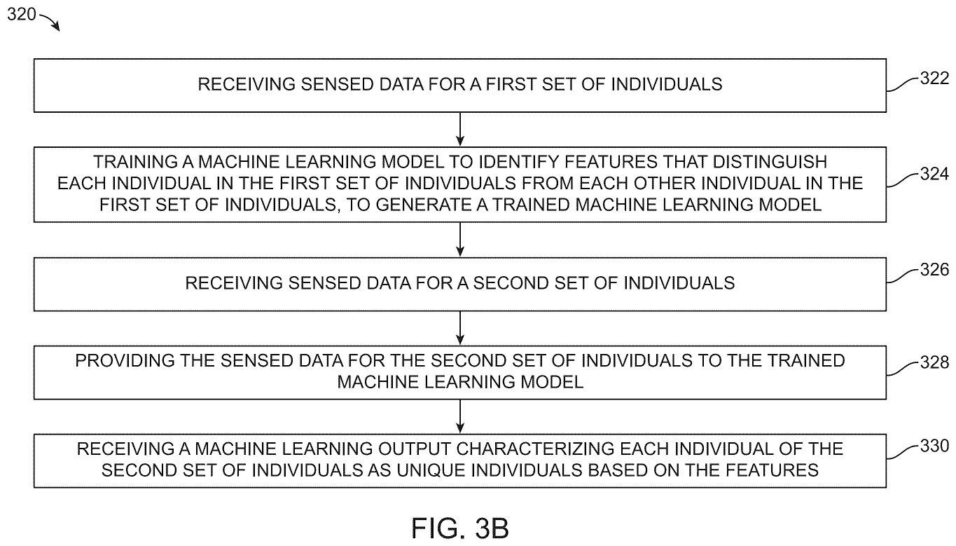

According to an implementation, the features used to identify unique individuals may be extracted from a machine learning model. FIG. 3A shows a flowchart 300 for extracting features from a machine learning model. At step 302, sensed data may be received for a first set of individuals. The sensed data may be sensed by any applicable sensing device with one or more sensors, such as the production devices or test devices disclosed herein. For example, the sensed data may be sensed using a wearable insole device. The sensed data may be raw data output by one or more sensors of the given sensing device. It will be understood that the sensing device may be a single component (e.g., having a single housing) or may be part of a system (e.g., a gait lab) that includes multiple components (e.g., having multiple housings). The raw data may be data that is not manipulated, filtered, or otherwise analyzed such that it may retain signal properties as output by the one or more sensors of the sensing device.

The sensed data may be sensed while each individual in the first set of individuals performs a sensing activity. The sensing activity may be any applicable action, lack of action, or the like that may be performed by each respective individual. For example, the sensing activity may be a walk, a step, a run, a jog, a movement, a reaction, or the like.

At step 304, a machine learning model may be trained to identify features that distinguish each individual in the first set of individuals from each other. A trained machine learning model may be generated based on the features. For example, the sensed data may be collected while each of the first set of individuals perform a walk while wearing a wearable insole device. Accordingly, the sensed data may include attributes about each individual while performing the walk and may include, for example, pressure data, acceleration data, variation in pressure during the walk, points where pressure is applied during the walk, and/or the like, as sensed by a plurality of sensors within the wearable insole device. The machine learning model may be, for example, a neural network based model (e.g., a convolutional neural network) configured to identify features in data, and may include further architecture, e.g., connected layer(s), neural network(s), weight(s), bias, node(s), etc., configured to determine a relationship between the identified features.

Accordingly, the features that distinguish each individual in the first set of individuals may be incorporated in the respective layers, networks, weights, biases, nodes, etc. of the trained machine learning model. The features may be or may be based on components or differences in the sensed data for each individual. For example, the features may be properties of a given signal or combination of signals (e.g., a combination of force data from multiple sensors over time, acceleration values, force distribution values, or the like). The properties of a given signal or combination of signals may be, for example, frequency, amplitudes, wavelengths, correlations between signals, correlations over time, patterns, or the like of the signals or one or more transformations of the signals. For example, the features may be components or differences in analyzed (e.g., transformed, filtered, amplified, etc.) signals derived based on the sensed signals.

At step 306, features identified by training the machine learning model may be extracted from the machine learning model. The features may be extracted by generating one or more outputs based on one or more trained machine learning model components such as connected layer(s), neural network(s), weight(s), bias, node(s), etc., of the trained machine learning model. For example, the configurations of the machine learning model components, as determined based on training the machine learning model, may be extracted from the machine learning model. The extraction may be processed by one or more processors, software, firmware, or the like that may have access to the machine learning model components. For example, the machine learning model components may be stored in a memory or storage and a processor or other component may access the memory or storage to extract the configurations. Accordingly, the configurations may be used to determine the features to identify how the machine learning model was trained to identify unique individual signatures.

According to implementations of the disclosed subject matter, the extracted features may be validated based on a second set of individuals. Sensed data for the second set of individuals may be received from a sensing device. The sensing device may be the same as or similar to the sensing device used to receive sensed data for the first set of individuals at step 302. The sensed data for the second set of individuals may be provided to the trained machine learning model. The trained machine learning model may generate a machine learning output based on the sensed data for the second set of individuals and the features used to train the machine learning model. The machine learning output may be received (e.g., at a processor) and may include a categorization of each individual in the second set of individuals based on the features. Accordingly, the machine learning output may distinguish each individual based on respective attributes related to the features for each individual.

According to an implementation, a characterization score may be determined based on the extent to which each individual in the second set of individuals is characterized as a unique individual based on the features. For example, if the characterization score may be relatively lower if the machine learning output includes overlap between two or more individuals' feature attributes and may be relatively higher if the machine learning output includes no overlap between any of the individuals' feature attributes. The characterization score may be compared to a pre-determined, input, or dynamically determined characterization threshold. If the characterization score meets or exceeds the characterization threshold, then the features may be validated as features that can be used to identify unique individual signatures.

FIG. 3B shows a flowchart 320 for characterizing individuals as unique individuals based on machine learned features. At step 322, sensed data for a first set of individuals may be received. At step 324, a machine learning model may be trained to identify features that distinguish each individual in the first set of individuals from each other individual in the first set of individuals. A trained machine learning model may be generated based on the training. At step 326, sensed data for a second set of individuals may be received. At 328, the sensed data for the second set of individuals may be provided to the trained machine learning model. At 330, a machine learning output may be received that may characterize each individual of the second set of individuals as unique individuals based on the features. Accordingly, the machine learning output may distinguish each individual in the second set of individuals, based on respective attributes related to the features for each individual.

Each block in the flow diagram of FIG. 1A or 2A, or flowcharts of FIGS. 1B, 2B, 3A, and 3B can represent a module, segment, or portion of program instructions, which includes one or more computer executable instructions for implementing the illustrated functions and operations. In some alternative implementations, the functions and/or operations illustrated in a particular block of a flow diagram or flowchart can occur out of the order shown in the respective figure.

For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

In various implementations disclosed herein, systems and methods are described for using machine learning to validate a test device and/or for validation of a machine learning model. By training a machine learning model, e.g., via supervised or semi-supervised learning, to learn associations between training data and ground truth data, the trained machine learning model may be used to validate one or more test devices.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, extreme gradient boosting (XGBoost), random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

As discussed herein, machine learning techniques adapted to validate a model and/or validate a test device, may include one or more aspects according to this disclosure, e.g., a particular selection of training data, a particular training process for the machine learning model, operation of a particular device suitable for use with the trained machine learning model, operation of the machine learning model in conjunction with particular data, modification of such particular data by the machine learning model, etc., and/or other aspects that may be apparent to one of ordinary skill in the art based on this disclosure.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine learning model, e.g., compare the output of the trained model with the ground truth for that portion of the training data to evaluate an accuracy of the trained model. The training of the machine learning model may be configured to cause the machine learning model to learn associations between training data and ground truth data, such that the trained machine learning model is configured to determine an output in response to the input data based on the learned associations.

In various implementations, the variables of a machine learning model may be interrelated in any suitable arrangement in order to generate the output. For example, in some embodiments, the machine learning model may include image-processing architecture that is configured to identify, isolate, and/or extract features, geometry, and or structure in one or more of the medical imaging data and/or the non-optical in vivo image data. For example, the machine learning model may include one or more convolutional neural network (“CNN”) configured to identify features in data, and may include further architecture, e.g., a connected layer, neural network, etc., configured to determine a relationship between the identified features in order to determine a location in the data.

In some instances, different samples of training data and/or input data may not be independent. Thus, in some embodiments, the machine learning model may be configured to account for and/or determine relationships between multiple samples.

For example, in some embodiments, the machine learning models described in FIGS. 1A-2B may include a Recurrent Neural Network (“RNN”). Generally, RNNs are a class of feed-forward neural networks that may be well adapted to processing a sequence of inputs. In some embodiments, the machine learning model may include a Long Shor Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of non-optical in vivo images as input, and generate a sequence of locations, e.g., a path, in the medical imaging data as output.

According to implementations of the disclosed subject matter, a wearable insole devices (e.g., a test device) may identify disease patterns comparable to gait measurements obtained in a traditional clinical laboratory (e.g. a production device) and may provide additional in-depth data types (e.g., features) that may identify subject-specific gait patterns. Gait analysis and related implementations are further disclosed herein. However, it will be understood that although gait analysis and related disclosure are provided as examples, the techniques disclosed herein (e.g., those related to gait analysis) are not limited to a single type of analysis, condition, device, or the like. For example, the implementations disclosed herein related to gait analysis and related discourse may be applied to or using any other analysis, condition, device, such as heart conditions, heart devices, biometric devices, biometric sensors, motion sensing devices, muscle related devices and/or conditions, bone related devices and/or conditions, organ related devices and/or condition, neurological related devices and/or conditions, electricity based devices and/or conditions, sensory based devices and/or conditions, or the like or a combination thereof.

As further discussed herein, biomechanical gait analysis can inform research and clinical questions such as detecting gait-related injury or disease and monitoring patient-specific recovery patterns. However, there are major limitations with such analysis conducted at gait labs, which require on-site patient assessments, trained specialists, and collect force and video data requiring specialized analytical techniques to fully interpret. Wearable insole devices may offer patient-centric solutions to this problem. A wearable insole device may measure disease-specific gait signatures virtually identically to the clinical, gait-lab gold standard of force plates. As further disclosed herein, a machine learning model, trained only on force plate data, is highly predictive not only on knee injury and control subjects force plate data (auROC=0.86; auPR=0.90) but also on a separate, independent wearable insole device dataset containing control and knee osteoarthritis subjects (auROC=0.83; auPR=0.86). Accordingly, the machine learning model trained based on a production device (e.g., a gait lab) can be used to validate a test device (e.g., a wearable insole device), as discussed herein.

Additionally, other data types provided by wearable insole devices, such as derived gait characteristics and raw sensor time series data may be analyzed. These different data types may inform key biological and endpoint-relevant questions about derivation of gait disease signatures, and, as further discussed herein, a single stride of raw sensor time series data may be used identify an individual's walk (e.g., an individual's signature as discussed in reference to FIGS. 3A and 3B). Accordingly, implementations of the disclosed subject matter provide alternatives to production devices and related tests such as traditional gait analysis methods, provide additional uses based on wearable insole device data analysis pipelines, and support clinical development of test devices such as via at home gait assessments, with the potential to improve the ease, frequency, and depth of patient monitoring.

Biomechanical gait analysis may inform clinical practice and research by linking characteristics of movement or gait with neurological or musculoskeletal injury or disease. However, there are limitations to the analyses conducted at gait labs as they require onerous construction of force plates into laboratories mimicking the lived environment, on-site patient assessments, as well as requiring specialist technicians to operate. Wearable devices, such as digital insoles, may offer patient-centric solutions to these challenges. As discussed herein, a digital insole may be used to measure osteoarthritis-specific gait signatures. The gait signature results using the digital insole may be similar results to the clinical gait-lab standard. A machine learning model may be trained on force plate data collected in participants with knee arthropathy and healthy controls. The model may be predictive of force plate data from a validation dataset (e.g., approximate values of area under the receiver operating characteristics curve (auROC)=0.86; area under the precision-recall curve (auPR)=0.90) and of a separate, independent digital insole dataset including control and knee osteoarthritis subjects (e.g., approximate values of auROC=0.83; auPR=0.86). Additionally, a single stride of raw sensor time series data may be accurately assigned to each subject such that, using digital insoles, individuals (e.g., including healthy individuals) may be identified by their gait characteristics. Although analysis related to gait analysis is generally described herein, it will be understood that the gold standard verses wearable device analysis discussed herein may be applicable to any assessment and is not limited to gait analysis.

As discussed herein, gait assessment plays several roles in clinical practice and research for neurological and musculoskeletal diseases: diagnostic workup; guiding treatment selection and measuring response; assessment of gait and balance pathophysiology, and/or the like. Traditional gait using a production device setting may be assessed in-clinic under the supervision of a physician, typically in a specialized gait lab with a force platform and/or motion tracking system. Gait labs use equipment that enables the creation of extensive models of human movement. Such equipment may include force plates to measure ground reaction force (GRF), cameras to generate video analysis for enabling mapping of an individual skeletal architecture, and electromyography sensors to measure muscle activity during movement. While applications of gait labs are diverse, in the context of a clinical trial setting for endpoint development, these detailed models of an individual's gait may not be required, and stand-alone components of the gait lab such as force plates may provide sufficient disease-relevant information.

A force plate(s) (e.g., gait mats) produces electrical signals that can be processed into three components of force (vertical, anterior-posterior, and medio-lateral), as well as the derived characteristic, center of pressure (COP) in the x/y direction. These signals provide information on gait characteristics, postural stability as well as direction, strength, duration of stance phase, and duration of motor activities during motion. Clinically, the force plate component of a gait lab is useful as it can provide insight into a patient's neuromuscular function and can guide diagnosis for disorders such as Parkinson's disease or progressive supranuclear palsy, can provide insight into disease progression and severity as shown with multiple sclerosis and/or osteoarthritis patients, and can identify patients with elevated falls risk by examining gait variability and balance. Drawbacks of force plate analysis include a requirement for specialists to interpret the results, inconvenience for patients in populations where required infrastructure is lacking, insurance and other monetary considerations for a doctor visit, operational costs of maintaining a staffed gait laboratory, and lack of ability for passive monitoring to capture patients' everyday activities. Other instrumentation like a force-instrumented treadmill can measure both GRF and kinematic data, however these tools suffer from similar limitations. However, such production devices and related systems can inform understanding of biology. For example, they may be used to determine that GRF is not correlated with kinematic tibial load metrics, which may indicate that a wearable sensor may be used for analysis similar to the production devices.

As discussed herein, test devices may be one or more wearable insole devices that can assess gait characteristics in controlled and free-living environments. Validated wearable insole devices may provide relevant aspects of the gait lab without the need of a clinical setting. Gait measurements with test devices may be used to provide or generate information that is comparable to a production device such as a gait lab, in a more user-friendly and patient centric manner. As discussed herein, a wearable insole device may be a smart or digital insole device that can quantitatively characterize gait and motion, to determine device usability, data quality, and ability to detect disease signals. An example single wearable insole device may include 25 vertical plantar pressure sensors that assess force, an accelerometer (e.g., a trail-axial accelerometer) that measures acceleration, and/or a gyroscope that measures orientation and angular velocity, for a total of 25 measurements on each foot. Each sensor may capture data at 100 Hz, and a variety of clinically relevant spatial and temporal gait characteristics may be calculated based on the sensed data. A digital insole may compute GRF in a similar manner as a force plate, generating comparable data outputs. Collectively, the data generated from these insoles may provide rich gait phenotyping information to characterize gait in patients with a broad range of neurological and musculoskeletal diseases. For example, in addition to the GRF data, derived gait characteristics summarizing an individual subject's walk and raw sensor time series data may be obtained using the digital insole wearable device disclosed herein. It will be understood that the wearable insole device described above is an example only, and that a given test device may be configured in any applicable manner and may include any applicable number and/or types of sensors.

The use of wearable insole devices for clinical uses presents a set of analytical challenges. Such use may improve diagnosis and monitoring of treatment responses, as well as support the development of meaningful digital biomarkers. Production devices used in current clinical practice, such as force plates, suffer from limitations such as containing dense raw sensor time series data with non-linear relationships that make analysis and interpretation challenging. Test device based analysis such as wearable sensor data analysis pipelines suffer from even greater knowledge gaps as there are a lack of well-established analytical methods for this field compared to other biomarker data types. Given that test devices (e.g., DHTs) intend to both complement and/or enhance gold standard approaches, analytical techniques disclosed herein both compare test devices (e.g., wearable insole devices) to their production device (e.g., gold standard device) counterparts and expand upon gold standards by providing greater biological intuition and medical interpretation of diagnosis or disease which may be used for digital endpoint development. Techniques disclosed herein allow understanding of optimal analytical approaches for intended clinical questions.

According to implementations of the disclosed subject matter, machine learning may be used as a tool to evaluate the digital biomarker quality and consistency, as well as how well data generated from test devices can be used to answer clinical questions. Techniques disclosed herein are directed to selection of appropriate modeling modalities for a particular clinical question. For example, when training classical machine learning models, a bias-variance trade-off may be considered. For bias-variance trade-off, a goal may be to include data that is rich enough to capture underlying prognostic or disease signals yet simple enough avoid overfitting such that these signals do not reproduce in an unseen disease population. An advantage of deep learning modeling techniques disclosed herein is that they can remain data-rich while avoiding overfitting. The selection of classical machine learning versus deep learning methods disclosed herein can be influenced by the structure and size of the data. For example, deep learning models are better suited to handle complex data types, such as raw sensor time series, but typically require larger datasets than classical statistical or machine learning methods. Finally, clinical test device (e.g., wearable insole device) data may be collected over several seconds to minutes, and longer in passive monitoring settings. Therefore appropriate application of large dataset processing, as disclosed herein, is important. Collectively, data type, size, and model selection are key components of a comprehensive test data analysis (e.g., wearable sensor data analysis) pipelines. Techniques disclosed herein for clinical research pipelines that generate enormous amounts of heterogeneous data (clinical, biomarker, and digital) are implemented in view of such issues.

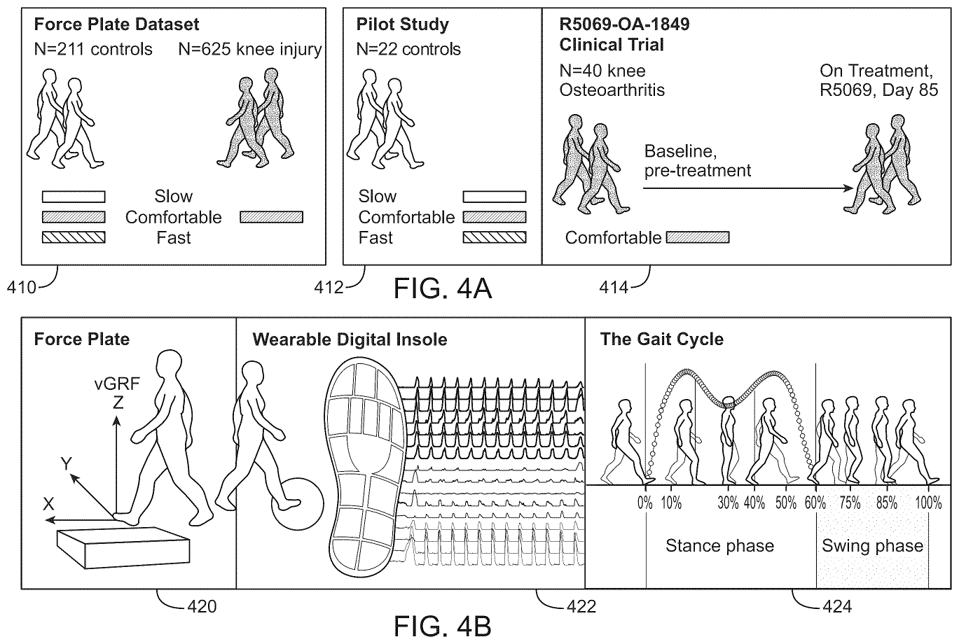

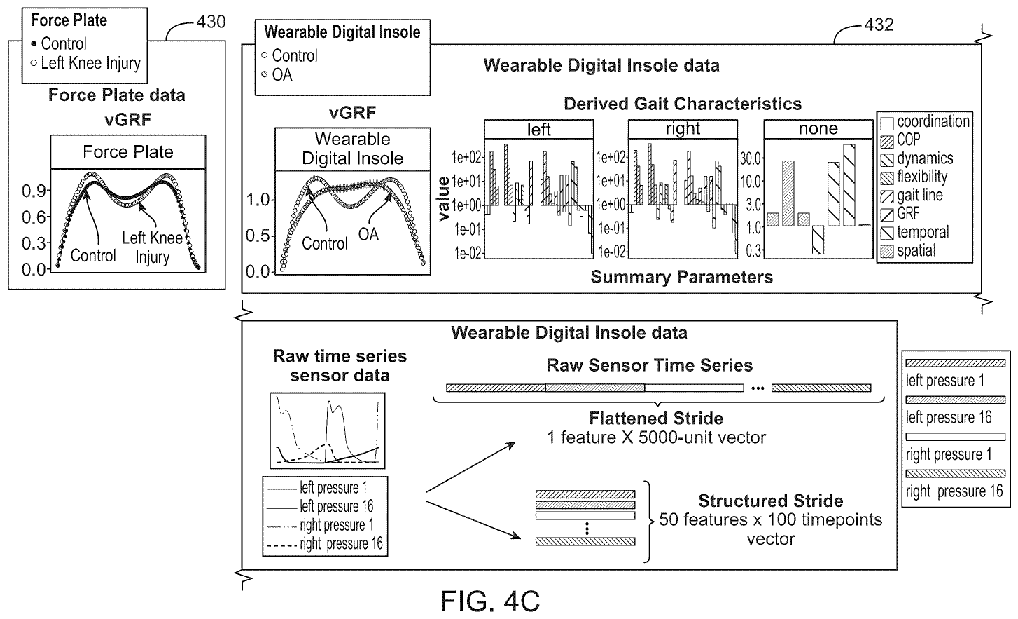

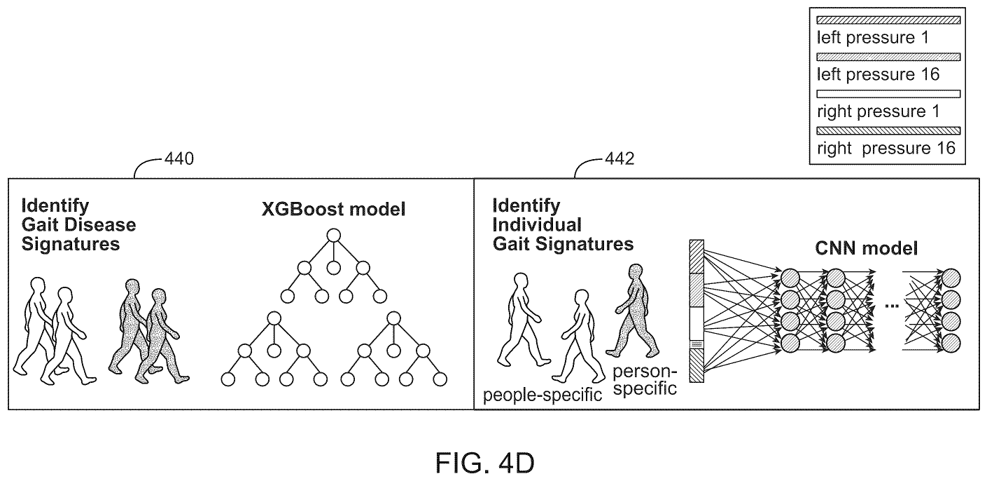

Techniques disclosed herein resolve such issues with an integrated analysis of production (e.g., gold standard) and test device (e.g., wearable insole device) data. According to an example further disclosed herein, three datasets that used either force plates (production device) or wearable insole devices (test device) in healthy controls and patients with a knee injury or knee osteoarthritis (OA) (target patients). The first dataset includes force plate vGRF data from control participants and target knee injury patients. The second dataset includes control participants from a pilot study evaluating a test wearable insole device. The third dataset includes participants who used the test wearable insole device from a knee OA clinical trial for a pain therapeutic. FIGS. 4A-4D show an integrated analysis of these various data sources, as further disclosed herein. The analysis shown in these figures addresses two key clinical and research implementations using machine learning approaches. In the first implementation, signatures of gait disorders in a biologically meaningful way are identified. In the second implementation, individual-specific gait patterns from walks or from a single stride, which would suggest at the richness of collected data are identified. Accordingly, techniques disclosed herein result in better clinical design and analysis with test devices (e.g., wearable insole devices) that measure human gait and provide a framework for meaningful clinical interpretation of these data.

The techniques disclosed herein, including the example provided herein, demonstrate that assessment of vertical GRF (vGRF) using state-of-the-art, research-grade force plates can be achieved with less expensive, more accessible, biosensor insoles that allow remote detection. The vGRF data may be assessed from three datasets collected with a wearable device, such as a digital insole, in subjects with knee arthropathy and control subjects to establish criterion validity of the digital insole, as compared to the force plate clinical standard. Using force plate measurements, a machine learning framework for detection of knee arthropathy status may be generated. The model may be validated using independent datasets collected with a wearable device such as a digital insole.

In the example provided herein, derived gait characteristics and raw sensor time series data from the digital insole were used for the analysis. In another example, three datasets that used either force plates or digital insoles in healthy controls and subjects with knee arthropathy were used for the analysis. The first dataset includes vGRF data collected with a force plate system, in control subjects and subjects with knee arthropathy (e.g., including knee fractures, ruptures of the cruciate or collateral ligaments or the meniscus, and total knee replacements). The second dataset included control subjects from a pilot study evaluating the digital insole. The third dataset included patients with a specific knee arthropathy/knee OA who were part of clinical trial in which participants also used the digital insole. Through an integrated analysis of these various data sources, disease signatures for knee arthropathy may be identified and individual-specific gait patterns may be detected.

### Example 1

Implementations of the disclosed subject matter are disclosed herein with references to an example. It will be understood that the implementations disclosed herein are not limited only to the data, orders, or specifics disclosed in the examples. It will be further understood that the example includes implementations that can be applied generally to the subject matter disclosed herein.

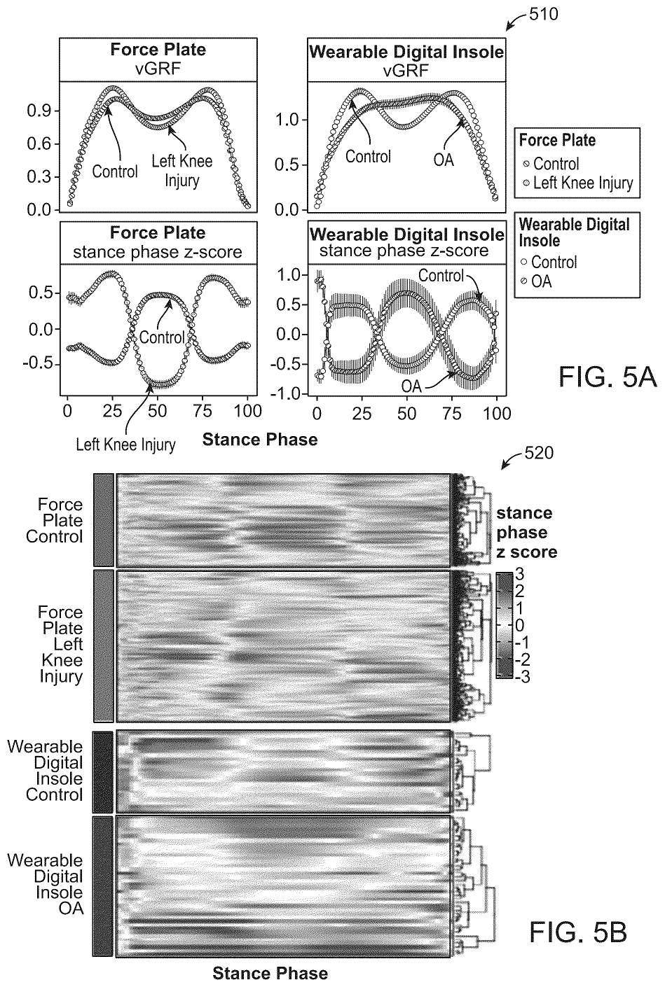

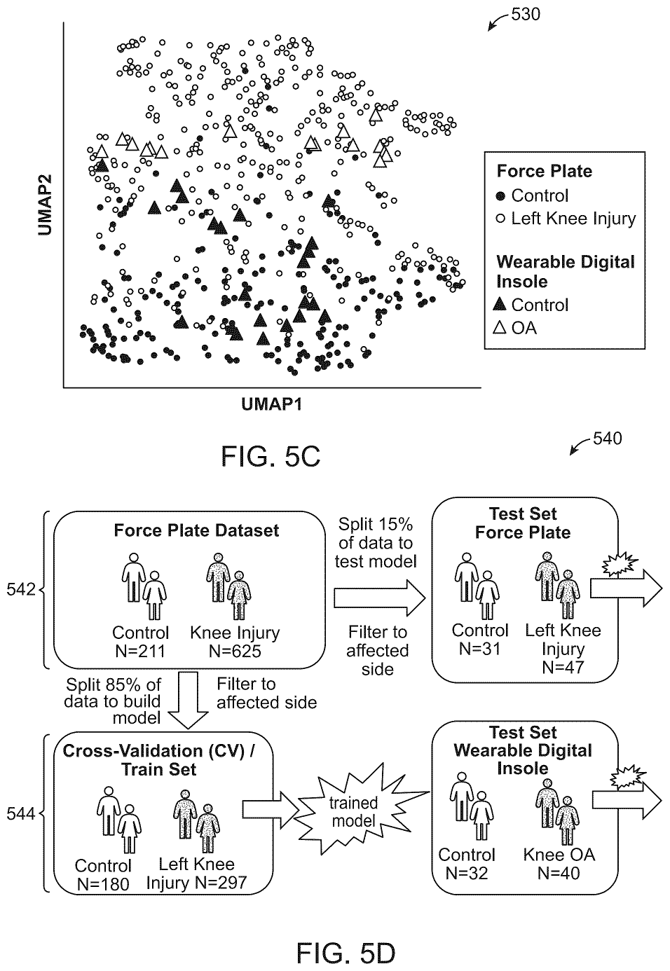

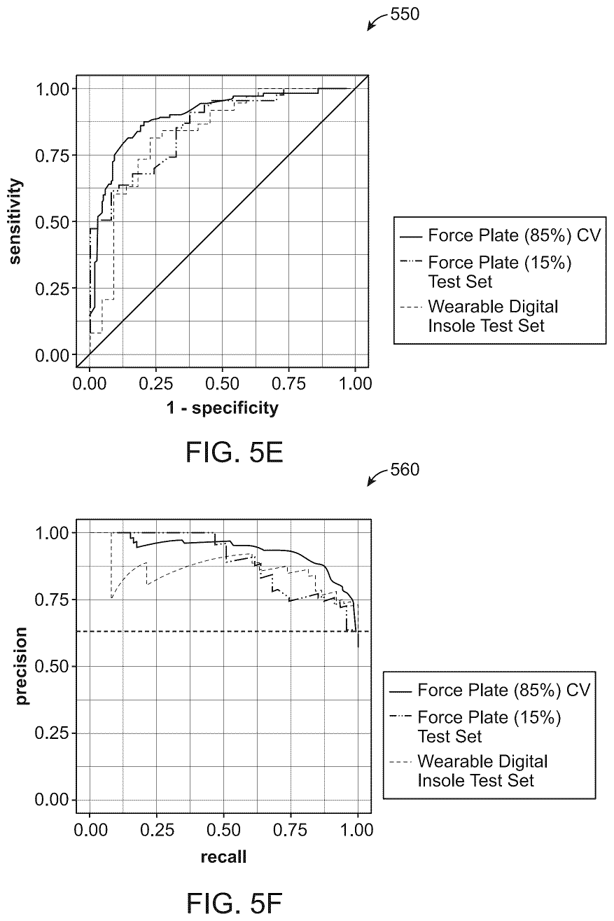

Disease signatures may be identified from vertical ground reaction forces in a platform agnostic-manner as described in reference to FIGS. 4A-4D and FIGS. 5A-5C. FIG. 4A shows dataset 410, dataset 412, and dataset 414. A (GaitRec) force plate dataset 410 (production device force plate data) includes N=211 healthy controls, who walked at three different walking speeds (slow, comfortable, and fast), and N=625 knee injury subjects, who walked at a comfortable walking speed (17). Dataset 412 is from a wearable insole device (test device) pilot study, where N=22 healthy controls walked at three different walking speeds (slow, comfortable, and fast). Dataset 414 is from a wearable insole device sub-study from a longitudinal clinical trial in knee osteoarthritis (OA), where N=40 knee OA subjects performed a 3-minute walk test (3MWT) at a comfortable walking speed at baseline (pre-dosing) and at day 85 (on treatment).

Table 1 below provides an overview of the models used in accordance with the example:

TABLE 1

| | | | Number of |
|---|---|---|---|
| Model | Train/Validation | Test | CV folds |
| Left foot | N = 471 total (85%) | N = 84 total (15%) | 5 folds |
| XGBoost | N = 297 left foot | N = 47 left foot injury | |
| vGRF | injury | N = 37 no injury | |
| model | N = 174 no injury | auROC = 0.857 | |
| | auROC = 0.905 | auPR = 0.895 | |
| | (0.033) | Digital insole vGRF | N/A |
| | auPR = 0.938 | (left) | |
| | (0.026) | N = 21 control | |
| | | N = 35 OA | |
| | | auROC = 0.815 | |
| | | auPR = 0.862 | |
| | Digital insole | N/A | leave-one- |
| | vGRF (left) | | out cross- |
| | N = 21 control | | validation |
| | N = 35 OA | | (LOOCV) |
| | auROC = 0.967 | | |
| | auPR = 0.979 | | |
| Right foot | N = 427 total (85%) | N = 76 total (15%) | 5 folds |
| XGBoost | N = 248 left foot | N = 44 left foot injury | |
| vGRF model | injury | N = 32 no injury | |
| | N = 179 no injury | auROC = 0.863 | |
| | auROC = 0.929 | auPR = 0.920 | |
| | (0.025) | Digital insole vGRF | N/A |
| | auPR = 0.954 | (right) | |
| | (0.020) | N = 21 control | |
| | | N = 35 OA | |
| | | auROC = 0.859 | |
| | | auPR = 0.909 | |
| | Digital insole | N/A | leave-one- |
| | vGRF (right) | | out cross- |
| | N = 21 control | | validation |
| | N = 35 OA | | (LOOCV) |
| | auROC = 0.951 | | |
| | auPR = 0.952 | | |
| Derived gait | N = 243 walks | N/A | leave-one- |
| characteristics | (N = 21 subjects) | | out cross- |
| XGBoost | control | | validation |
| | N = 73 walks | | (LOOCV) |
| | (N = 37 subjects) | | |
| | OA | | |
| | auROC = 0.9997 | | |
| | auPR = 0.9989 | | |
| Derived gait | N = 243 walks | N/A | leave-one- |
| characteristics | (N = 21 subjects) | | out cross- |
| without highly | control | | validation |
| correlated | N = 73 walks | | (LOOCV) |
| (rho > 0.7) | (N = 37 subjects) | | |
| walking speed | OA | | |
| characteristics | auROC = 0.998 | | |
| XGBoost | auPR = 0.993 | | |
| Flattened | N/A | N/A | leave-one- |
| strides | | | out cross- |
| XGBoost | | | validation |
| | | | (LOOCV) |
| Sensor models | N/A | N/A | N/A |

In accordance with Table 1, when constructing machine learning models with force plate derived vGRF data, a 5-fold cross validation is applied for the training/validation set. This full training/validation set is then used to construct a single model and applied to the test set. Subjects with knee injury on both joints are filtered, and there is only one vGRF observation per subject. Accordingly, the analysis of the force plate dataset for building a machine learning model is limited to those subjects with left or right knee injury (e.g., excluding subjects who had knee injury on both joints), thereby matching the clinical trial enrollment criteria (KL score≥2, index joint).

For instances where enough data is not available to perform cross validation, in the case of the digital insole derived data, XGBoost models are trained and assessed using leave-one-out cross-validation (LOOCV), where models are evaluated by iteratively leaving one subject out, building a model, and evaluating where that subject would be classified compared to the true result.

The choice of a model in machine learning applied herein may be based on one or more of the specific problem at hand, data characteristics, and/or performance requirements (e.g., for given scientific questions). An XGBoost model is used herein as a machine learning model and a 1D Convolutional Neural Network (CNN) is used for as a deep learning model.

The XGBoost model may be chosen over other models such as, for example, Support Vector Machine (SVM) and/or Random Forest (RF) for one or more reasons. For example, XGBoost, being a gradient boosting framework, may offer model performance and efficiency advantages. Gradient boosting algorithms, including XGBoost, may outperform other algorithms, such as for datasets where the relationship between the features and the target variable is complex or involves non-linear relationships, as is the case in gait analysis. In the case of the dataset discussed herein, the analysis is robust to the model choice and similar performances is observed with logistic regression and SVM approaches.

For deep learning, a 1D Convolutional Neural Network (CNN) is used. CNNs may be suited for this example use-case because they may excel in handling sequential data with temporal dependencies, such as time series data from our digital insoles. CNNs may automatically learn and extract key features, reducing the need for manual feature engineering, and they may be robust against shifts and distortions in the data.

In accordance with this example, the force plate vGRF dataset is first randomly split with 85% samples to be used for training/validation and the remaining 15% put aside as a hold-out test set. 5-fold cross validation is used to initially assess the model performance on the 85% training/validation set. This full training/validation set is then used to construct a single model and applied to both the 15% hold-out test set and the digital insole-derived vGRF data from this example. As one vGRF observation per subject in the vGRF datasets is used (both from force plates and from digital insoles), there were are repeated participants between the folds/splits.

FIG. 4B shows that both force plates (production device) at 420 and wearable insole device (test device) 422 produce different types of data. These data are compiled from data collected during stance and swing phases of a person's gait cycle at 424.

FIG. 4C shows various types of data produced by the production and test devices including vGRF data 430, derived gait characteristic data, and raw sensor time series data shown at 432.

FIG. 4D shows two analytical methods were used to evaluate these data.

XGBoost, a gradient boosting classifier, is used to analyze vGRF, derived gait characteristic, and raw sensor time series flattened stride data at 440. Clinical research implementations include the derivation of gait disease signatures of knee OA and investigation of the individuality and consistency of gait patterns. Two analytical techniques were used to evaluate the corresponding data. XGBoost, a gradient boosting classifier, was used to analyze vGRF, derived gait characteristics, and raw sensor time series flattened stride data at 442. A one-dimensional convolutional neural network (CNN) was used to analyze structured stride raw sensor time series data at 422.

FIG. 5A shows vGRF curves 510 derived from force plate (production device) and wearable insole device (test device) data for healthy controls, and knee injury and knee OA subjects, respectively. Left foot data are shown as mean of values (top panels) and mean of normalized z-scores (bottom panels) at each percent stance phase within each device and health status.

FIG. 5B shows vGRF curves for each individual's left foot shown as heat map 520 rows, after data was z-transformed to generate Z scores at each percent stance phase (as in FIG. 5A). As disclosed herein, the term “Z score” refers to a value of how many standard deviations given data is away from the mean. If a Z score is equal to 0, the data is at the mean. A positive Z score indicates that a raw score is higher than the mean average. A negative Z score indicates that a raw score is below the mean average. As shown in FIG. 5B, Rows are hierarchically clustered with each category of force plate controls, wearable insole device controls, wearable insole device knee OA subjects, and force plate left knee injury subjects.

FIG. 5C shows a UMAP dimensionality reduction 530 of the z-transformed left foot vGRF data. Each point represents a subject, and points are separated by phenotype, and shaped by device.

vGRF data collected from a wearable insole device (test device) may be compared to vGRF data from the clinical gold standard of force plates (production device). As shown in FIG. 4A, at dataset 410, vGRF dataset of subjects with knee injuries and healthy controls is received. The GaitRec force plate dataset provided N=211 controls who walked at a comfortable walking speed, in addition to a slow and fast walking speed, and N=625 knee injury subjects who walked at a comfortable walking speed. There were 297 subjects with left knee injury only and 248 subjects with right knee injury only.

From two separate studies, data from a wearable insole device was collected as shown in FIG. 4A at dataset 412 and dataset 414. The first is a pilot study shown at dataset 412 of N=22 controls, who walked for −30 second walking intervals at a comfortable walking speed, in addition to a self-paced slow and fast walking speed. The second shown at dataset 414 is a clinical trial for a pain medication (NCT03956550) of N=40 subjects with OA of the knee who performed a 3-minute walk test (3MWT) at a comfortable walking speed with the wearable insole device. The 3MWT is performed at baseline (pre-treatment), as well as at day 85 post-treatment. For disease-relevant gait analysis, the data is restricted to baseline 3MWT. The participants in these two wearable insole device datasets are different than those profiled in the force plate dataset.

To visualize the vGRF data from all studies, vGRF curves from force plate and wearable insole device data are plotted, and qualitatively observed that the means of vGRF curves within each health status are similar across platforms, as shown in FIG. 5A. Subsequent normalization (z-score) within each platform, at each time-point along the stance phase, provides comparison of the different platforms on the same scale. Distinct patterns between control individuals and those with injured knees, when comparing the means of normalized vGRF curves are shown in FIG. 5A's lower panel and each individual's normalized curve is shown on a heat map 520 of FIG. 5B. Specifically, individuals with affected knees qualitatively look different than healthy controls with a “flatter” vGRF curve shape during the middle of stance phase, as well as a visually noticeable difference in pattern when represented on a heat map. It should be appreciated that the term “arthropathy” is used herein to encompass both knee injuries and OA. Using a dimension reduction approach (UMAP) with normalized vGRF data from each subject in two dimensions, subjects at a population level separated out by arthropathy status (knee injuries vs controls) rather than by measurement platform, as shown in UMAP dimensionality reduction 530 of FIG. 5C. Thus, despite performing analysis on data collected from both production devices and test devices at different sites, apparent disease-relevant patterns in the vGRF data are observed, especially with respect to how the test device (wearable insole device) data appears to recapitulate the force plate data.

Accordingly, as discussed herein in reference to FIGS. 1A and 1B, production device data can be used to validate test device data. Production device data (e.g., force plate data shown in FIGS. 4A and 5A-5C can be compared to test device data (e.g., wearable insole device data) to validate the test device.

A series of linear models may be fit to each point along a vGRF curve to investigate how a variation in the vGRF data may be partially explained by clinical and/or demographic characteristics of the participants. In this instance, arthropathy state (e.g., knee arthropathy or control), age, sex (e.g., male or female), and/or body weight may be covariates in the model. For each linear model, which may represent the sum of squares for each category compared to a total sum of squares as a percentage of variation explained by that component, a disease state may be a major contributor to a vGRF signal for a majority of the vGRF curve, with age, sex, and body weight contributing to a relatively smaller proportion of the variance. In this instance, an arthropathy state may be determined as a primary factor contributing to variation among participants to said signals.

Machine learning models trained on vertical ground reaction forces can be used to classify control versus knee injury (target condition) across different platforms as shown in FIGS. 5D-5F. FIG. 5D shows a schematic 540 of machine learning model building of training/validation and testing sets. Two XGBoost models, one for left knee injury (depicted) and one for right knee injury are generated. The full force plate vGRF dataset with both controls (comfortable walking speed) and left or right knee injury subjects (comfortable walking speed, excluding subjects with knee injury on both joints) are split 85% into training/validation datasets, and 15% into a hold-out testing set. A first model predicts control versus knee injury subjects using left foot data (of left knee injury subjects and all controls), and the second predicts using right foot data (of right knee injury subjects and all controls). These models were then applied on a separate, independent testing set of wearable insole device vGRF data with N=22 controls at section 542 and N=38 knee OA subjects at section 544. Section 544 shows an implantation in accordance with FIGS. 2A and 2B to validate a machine learning model based on first and second sub-sets of individuals.

FIG. 5E shows a receiver operating characteristic (ROC) curve 550 for XGBoost classification of force plate (85%) cross-validation (CV, training/validation) set, force plate (15%) hold-out test set, and the wearable insole device test set. FIG. 5F shows a precision-recall curve for XGBoost classification of the same groups shown in FIG. 5E.

Techniques disclosed herein may be used to determine how to optimally classify control versus target (e.g., knee OA) with test device (e.g., wearable insole device) data. The machine learning models disclosed herein may be used to quantitate how well force plate data can identify disease signatures of gait abnormalities, and to understand if a wearable insole device can detect these same signatures. To predict controls versus target knee injury using vGRF data, complete vGRF force plate dataset are divided into a train/validation set (85%) and a test set (15%). A gradient boosting machine learning model (e.g., an XGBoost model) is trained, to predict these classes, as shown in FIG. 5D. To evaluate the model, the area under the receiver operating characteristics (auROC) curve is quantitated, which may be a measure of classification success and describes model performance regardless of baseline likelihood for either class. In addition, the area under the precision-recall curve (auPR) is quantitated, which is used for evaluating datasets with class imbalances. Evaluation of the model indicate strong predictive power to classify control versus knee injury subjects using force plate data, both when evaluated using five-fold cross validation of the training/validation dataset, a method of assessing model performance (left knee: area under the receiver operating characteristics curve [auROC]=0.91 (sd=0.03), area under the precision-recall curve [auPR]=0.94 (sd=0.03); right knee: auROC=0.93 (sd=0.03), auPR=0.95 (0.02)). The predictive power was also strong when assessed on the hold-out test dataset, which was not used for training of the XGBoost model (left knee: auROC=0.86, auPR=0.90; right knee: auROC=0.86, auPR=0.92).

To further assess generalizability of the model and validate a test wearable insole device to measure vGRF similarly to a force plate, the model trained using vGRF force plate data is applied to the separate, independent dataset derived from test wearable insole device datasets for and/or studies on individuals with target knee OA on healthy controls. As observed in FIGS. 4A-4C, an assumption of this analysis is that target knee OA and knee injuries are indicated similarly by vGRF curves. It is shown that for a test wearable insole, a machine learning model trained only on production force plate data performs nearly as well (e.g., within a match threshold) on the digital insole data (left knee: auROC=0.82, auPR=0.86; right knee: auROC=0.86, auPR=0.91), as it did on the hold-out force plate test data, as shown in FIGS. 5D-5E. Accordingly, the machine learning model is not overfit, as it was validated based on an independent dataset collected from a different device at different physical locations and experimental conditions. Comparable results when training and testing on right foot vGRF data for right knee injuries are shown in Table 2.

TABLE 2

| | Receiver | | | | | | | |
|---|---|---|---|---|---|---|---|---|
| | Operating | | | | | | | |
| | Characteristic | | Precision - | | | | | |
| | Curve AUC | | Recall AUC | | F1 Score | | | |
| Per class binary | (auROC) | | (auPR) | | Controls | | Affected | |
| classification metrics | Left | Right | Left | Right | Left | Right | Left | Right |
| Force plate 5-fold | 0.905 | 0.929 | 0.938 | 0.954 | 0.77 | 0.83 | 0.87 | 0.88 |
| cross validation | (.033) | (0.025) | (.026) | (0.020) | | | | |
| Force plate test set | 0.87 | 0.863 | 0.895 | 0.920 | 0.71 | 0.63 | 0.74 | 0.80 |
| Digital insole test set | 0.815 | 0.859 | 0.862 | 0.909 | 0.72 | 0.72 | 0.80 | 0.77 |

Table 2 shows force plate vGRF control versus knee injury (arthropathies) XGBoost classification model evaluation statistics on left foot and right foot data. An XGBoost model is trained on 85% of the force plate dataset vGRF data to predict control or knee arthropathies (knee injury or knee osteoarthritis (OA)) classes, with left foot vGRF data used to predict left knee arthropathies and right foot vGRF data used to predict right knee arthropathies. The model is evaluated using five-fold cross validation, a held-back (or hold-out) force plate test set, and a wearable (or digital) insole device test set. auROC and auPR statistics are shown for the three models. F1 scores for each class for each model are also shown.

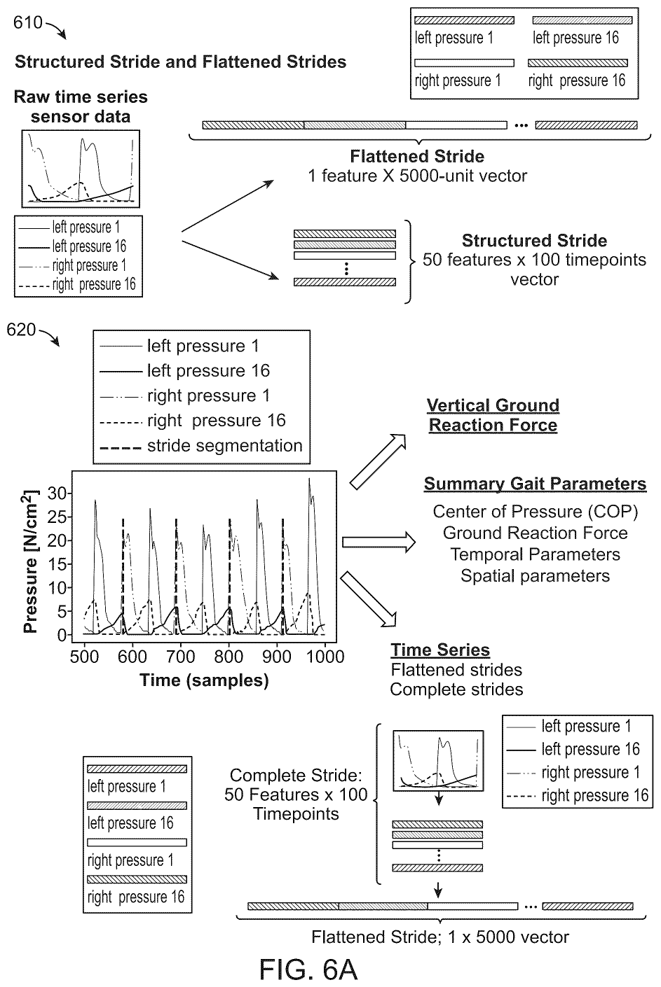

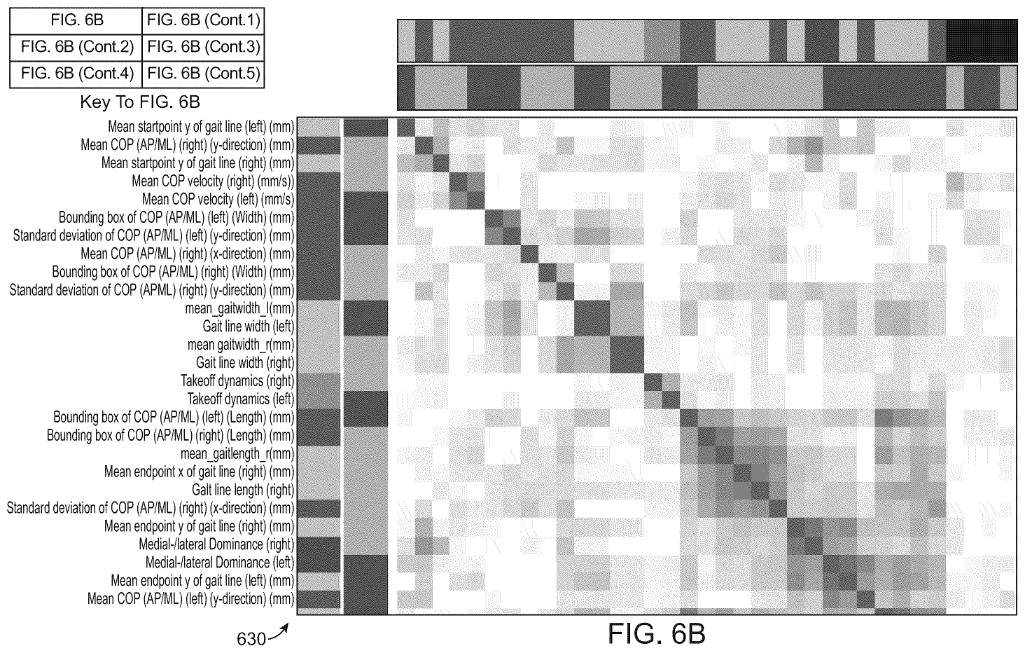

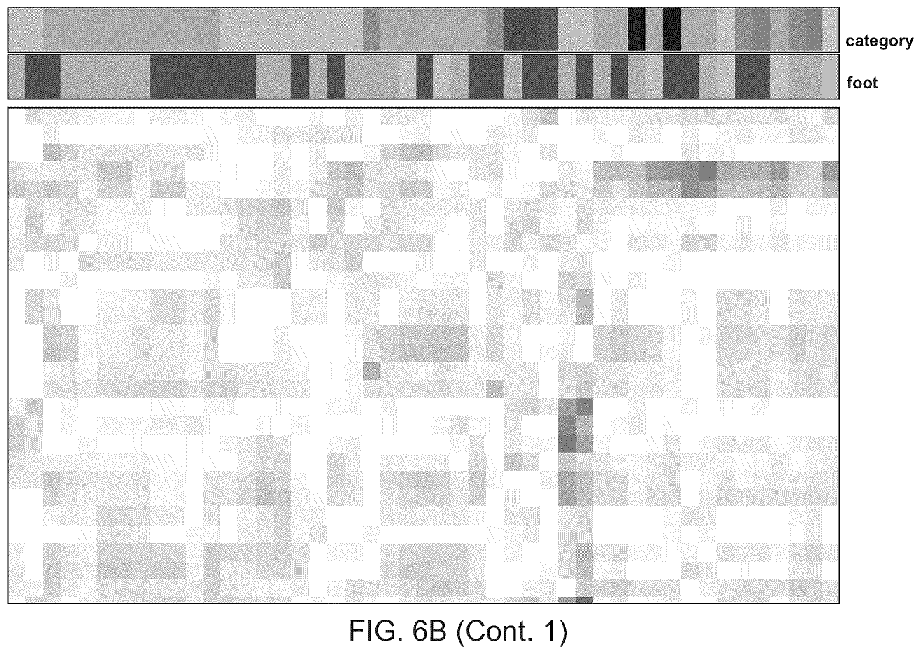

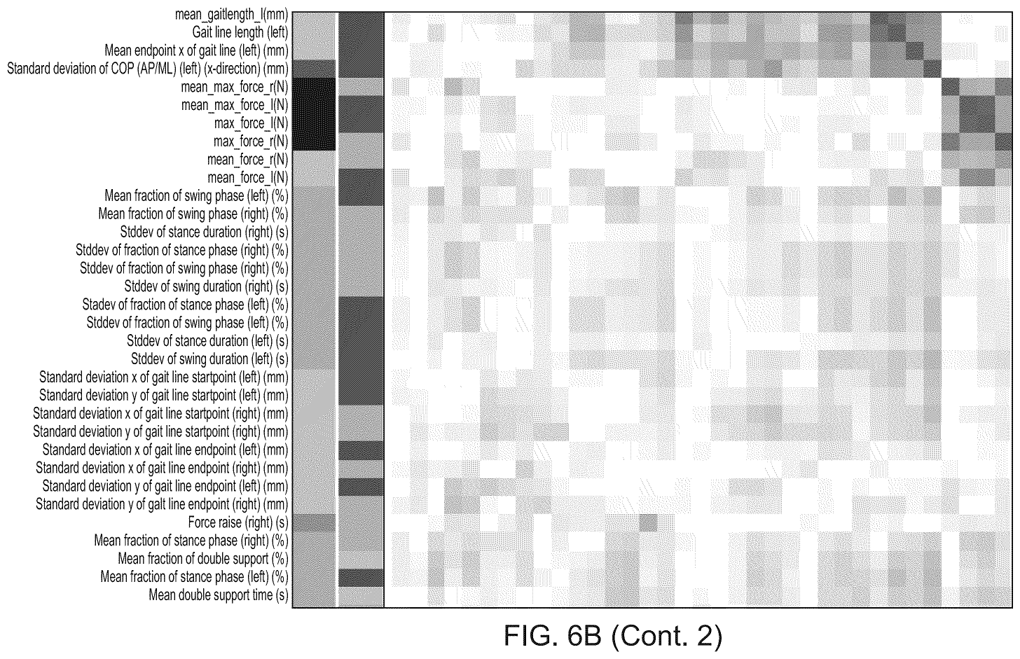

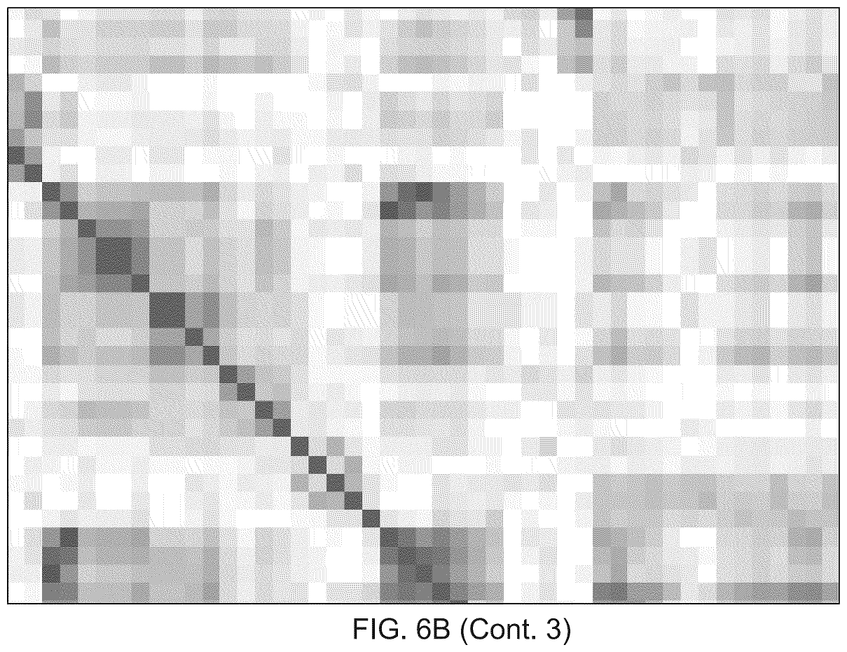

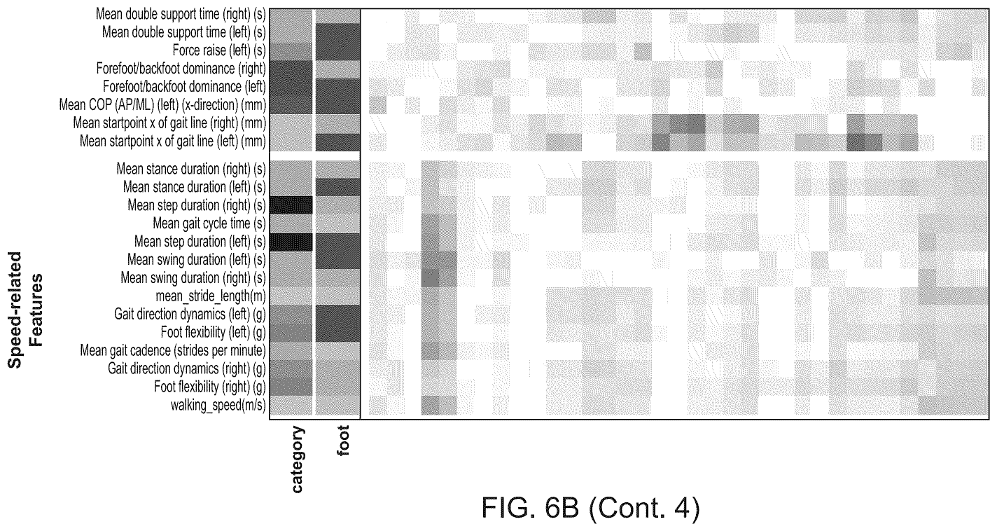

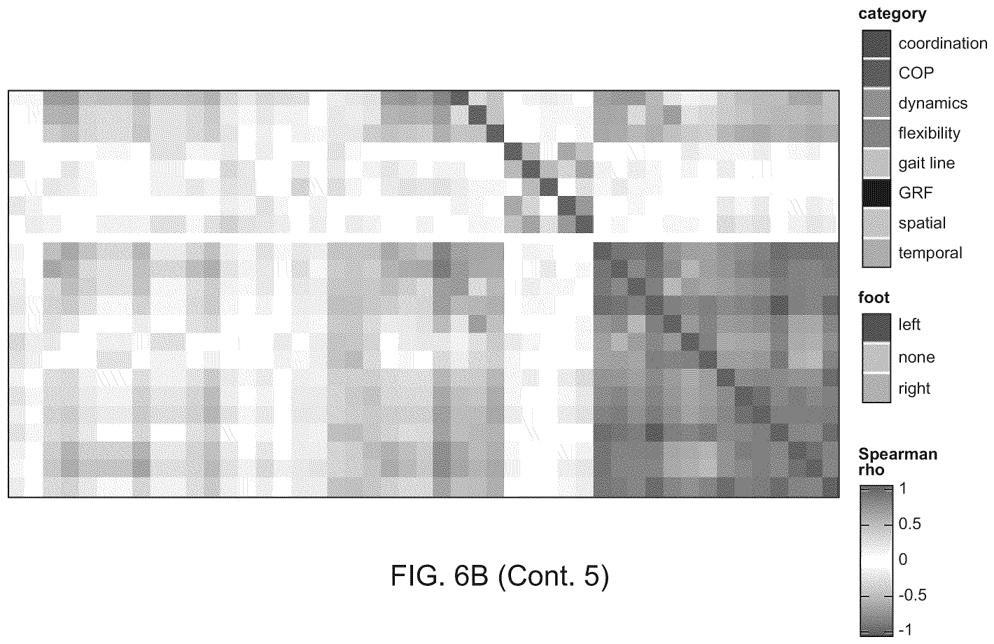

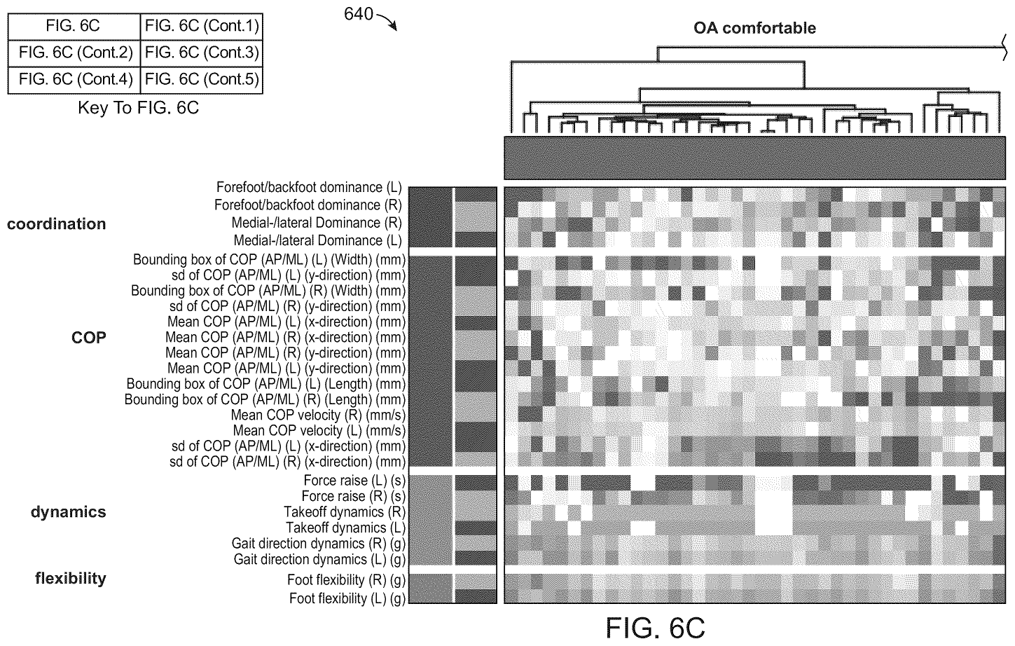

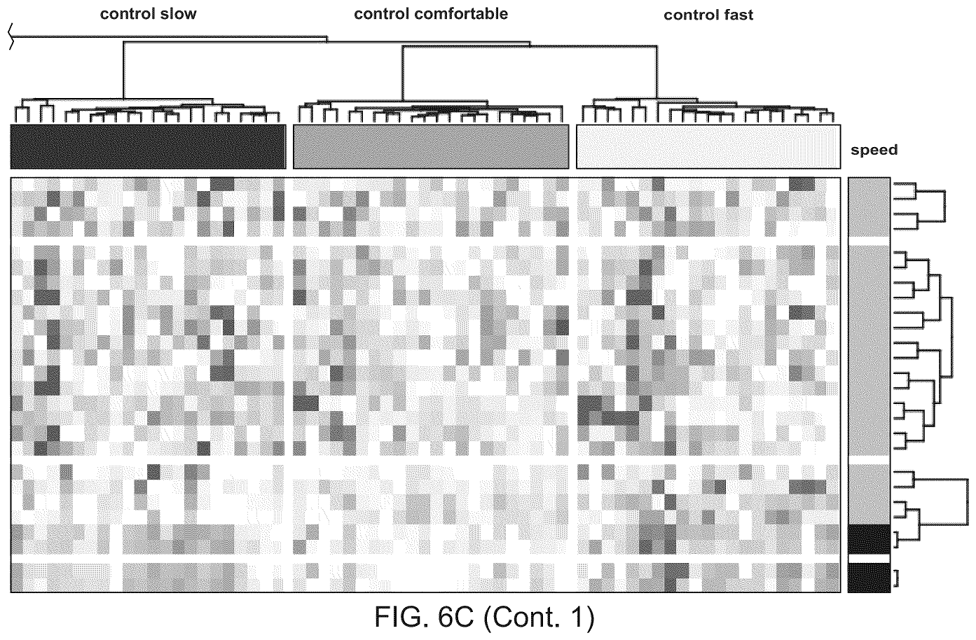

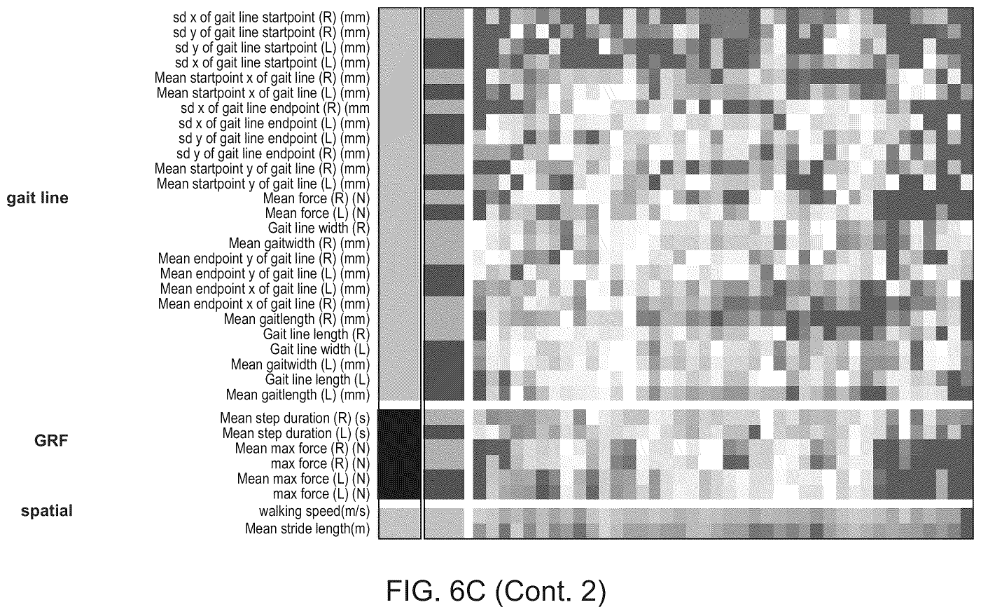

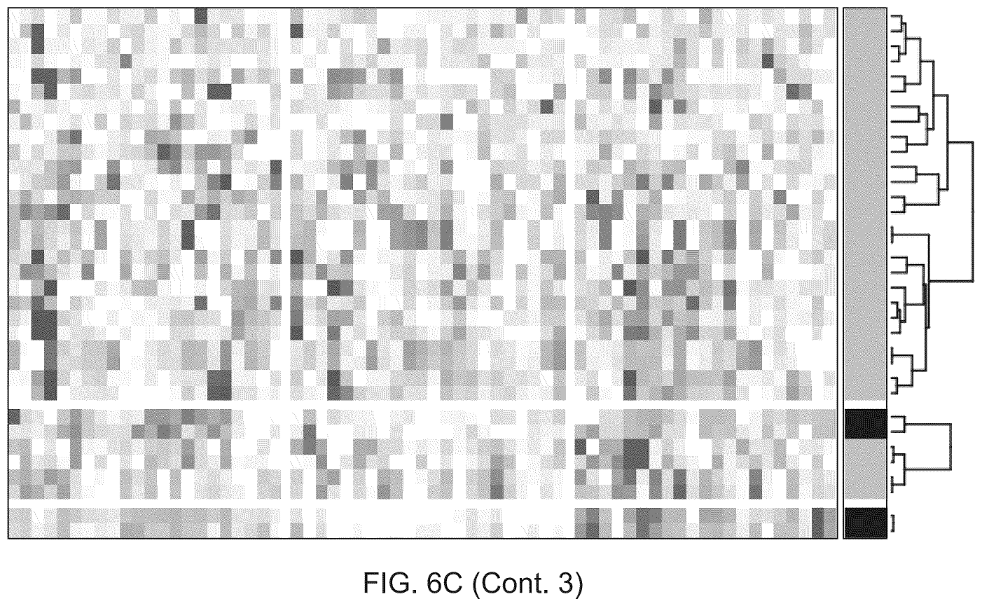

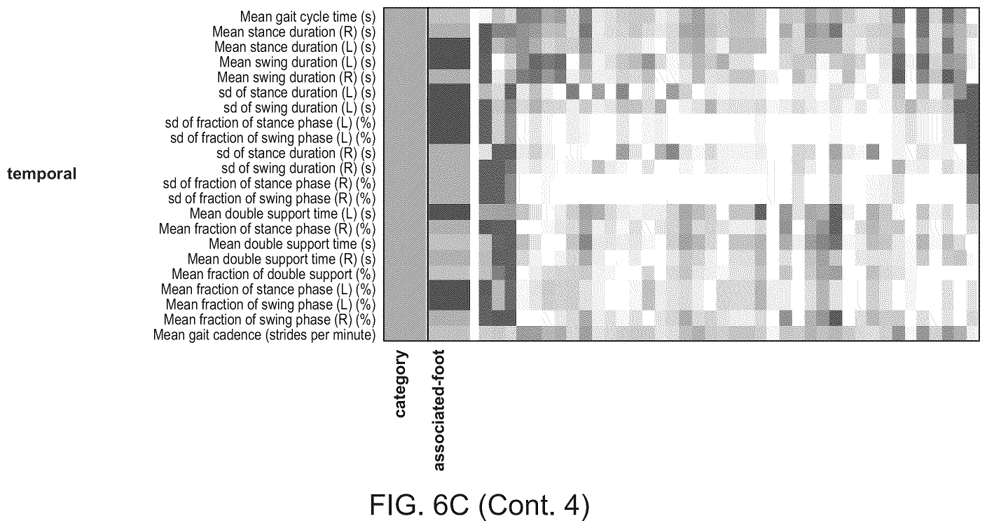

As shown in FIGS. 6A-6C, derived gait characteristics of a test wearable insole device outperform vGRF and raw sensor time series for disease classification. FIGS. 6A-6C show test wearable insole device summary parameters and time series sensor data, in addition to vGRF data. It should be understood that this analysis may classify whether a subject has knee arthropathy, rather than to determine a severity of arthopathy.

FIG. 6A shows a schematic 610 of raw sensor time series data from a test wearable (digital) insole device. Data may be processed from the test device in three ways: (1) vertical ground reaction forces, as shown in FIGS. 4A-4C (2) derived gait characteristics on force, spatio-temporal, and center of pressure aspects, and (3) raw sensor time series data from 50 sensors embedded across both test wearable insole devices. Each segmented stride of raw sensor time series data can be analyzed as is (structured strides) or collapsed (flattened strides), as shown in schematic 610 of FIG. 6A. As shown at 620, vGRF data, summary gait parameters, and time series data can be calculated from test data from the test wearable insole devices.

FIG. 6B shows a correlation matrix 630 of derived gait characteristics (parameters) of the test wearable (digital) insole device from all individuals in the pilot study, correlated against each other at the comfortable walking speed. Spearman correlation coefficients are computed and shown in a correlation matrix 630 ranging from −1 (perfect anti-correlation) to +1 (perfectly correlation). Each parameter has a spearman correlation coefficient of +1 with itself (red diagonal). The parameter, the foot from which it was generated, and its category are labeled on the left of the correlation matrix 630.

FIG. 6C shows a heat map 640 representation of the average of each of 82 test (digital) insole parameters (rows) across all walks for each patient (columns) from the pilot study. Parameter values are shown as normalized z-scores (bounded within ±3), calculated across all participants and walking speeds. The heat map 640 representation is split by the three walking speeds (slow, normal, fast), and columns are clustered within each walking speed using hierarchical clustering with Euclidian distances. The 14 parameters that are strongly correlated with walking speed are indicated on the right of the heat map.

One benefit of collecting gait data from a wearable insole device such as test wearable insole device is that potentially more comprehensive data may be measured because of the additional embedded sensors in the test wearable insole device, relative to a production force plate. In addition to the vGRF curves discussed above, both derived gait characteristics and raw sensor time series data can also be measured or derived from the 50 wearable sensors across both insoles, as shown in FIG. 6A. The derived gait characteristics represent clinically meaningful features to gait, like coordination, dynamics, gait line, ground reaction force, spatial, and temporal descriptors of walking, and are typically summarized over a period where a participant is asked to walk at a comfortable speed. Additionally, the raw 100-Hz sensor time series data is segmented into individual strides. The data is interpolated to obtain measurements for each sensor at 100 time-points along each stride. Time series data may measure stride components of force, angular rate, and orientation of the body. The shape of the raw sensor time series data may be evaluated as either structured strides of dimensions 50-by-100, or converted into flattened strides, where the time-points and sensors are concatenated into a single vector of length 5,000, as shown in FIG. 6A.

To determine how derived gait characteristics relate to each other, the values and their correlations are clustered across different walking speeds and disease status. Correlations within and between categories of parameters show that similar groups of parameters cluster together, as shown in FIG. 6B, including 14 derived gait characteristics that are found to be strongly correlated with walking speed (|Spearman rho|>0.7). It should be understood that other gait characteristics may be influenced by walking speed and those characteristics described and shown herein may include exemplary characteristics that are influenced to a greater relative degree by speed, defined by this threshold. In other words, these gait characteristics may represent a subset of characteristics most influenced by speed, defined by this threshold. Using heat map 640, where derived gait characteristics are normalized (z-scored) across both control and knee OA populations, distinct patterns between derived gait characteristics at different walking speeds and disease (arthropathy) status are observed.

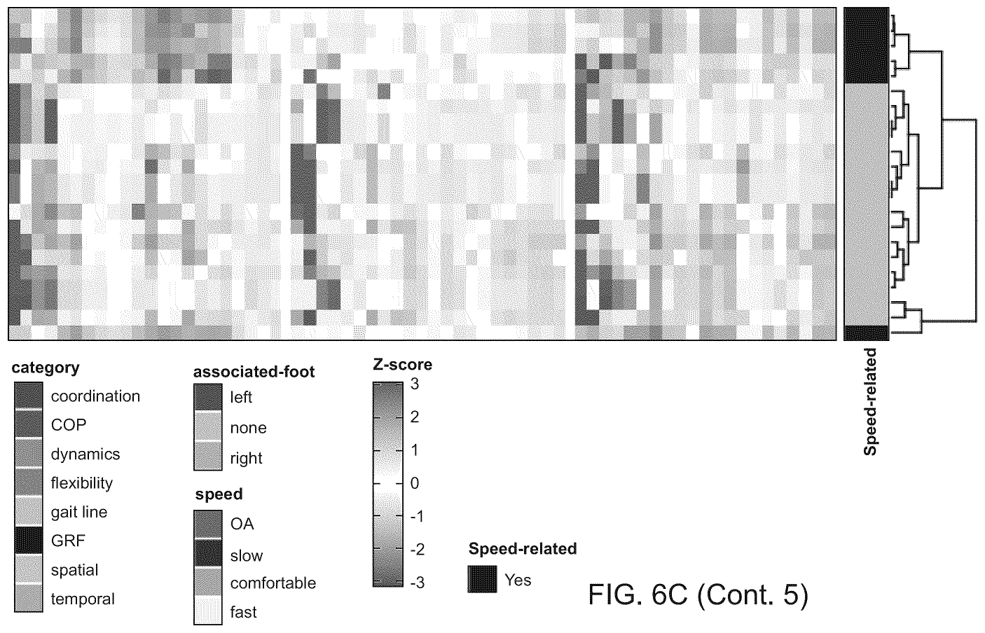

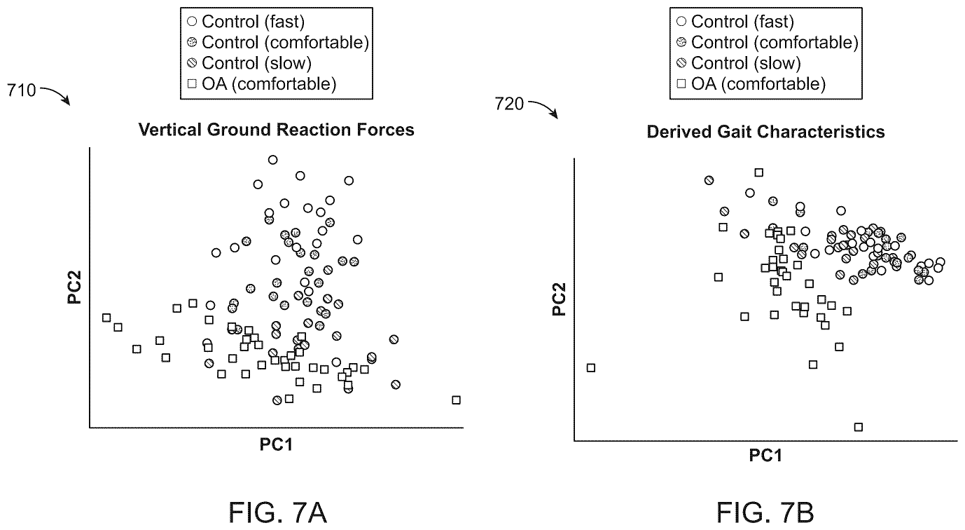

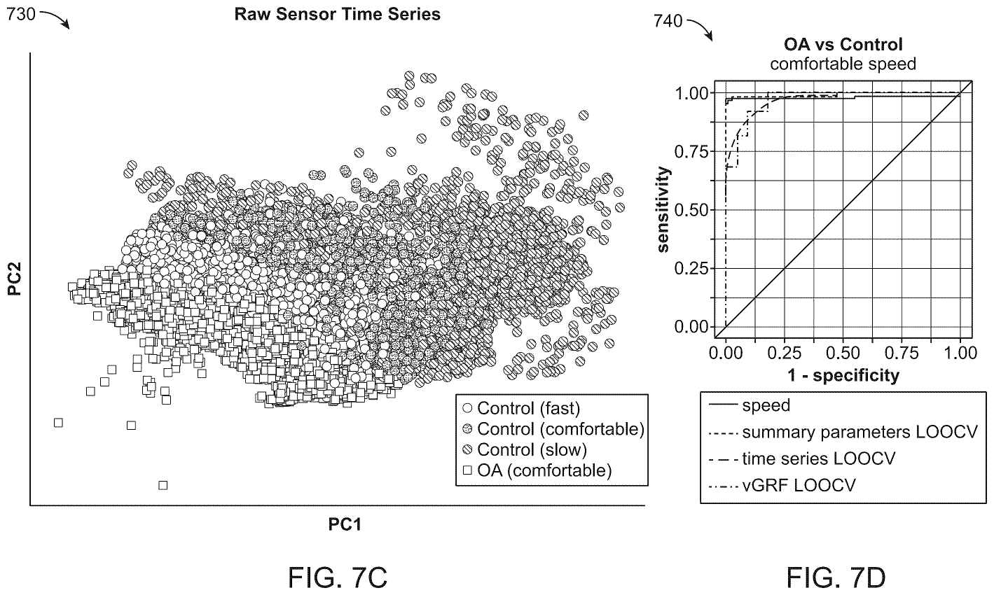

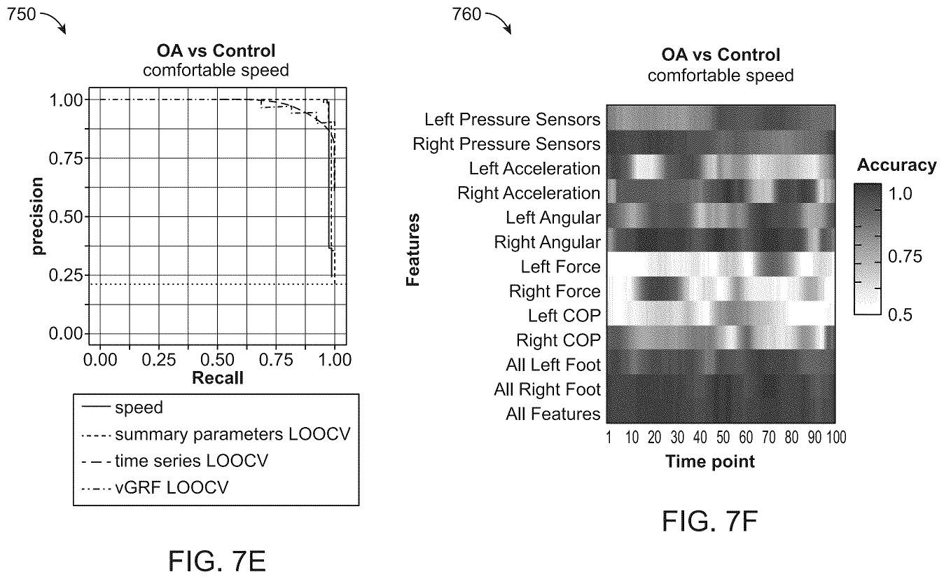

According to implementations, walking speed may be used as a sole parameter to group control and target knee OA subjects. To visualize the relationship with walking speed, principal component analysis (PCA) dimensionality reduction on each data type is generated, which shows that target knee OA anthropathy state can be observed on a continuum related to walking speed. Target knee OA is shown to be more strongly associated with walking more slowly as apparent across all data types, including vGRF, as further shown in FIG. 7A, derived gait characteristics shown in FIG. 7B, or raw sensor time series data shown in FIG. 7C.

FIGS. 7A-7E show different methods to analyze control vs target (OA) data with a test wearable insole device to generate refined classification of disease signatures. FIG. 7A shows PCA dimensionality reduction 710 of vGRF data from all walks of pilot study subjects and baseline target walks of knee OA clinical trial participants. Each dot represents data from a single subject at a given walking speed.

FIG. 7B shows a PCA dimensionality reduction 720 of derived gait characteristic data from the wearable (digital) insole device, without the 14 speed-correlated derived gait characteristics. FIG. 7C shows a PCA dimensionality reduction 730 of raw sensor time series of each stride from all walks. Each dot represents data from a single stride and repeat strides from the same participant are shown. FIG. 7D shows ROC curves 740 for target knee OA versus control (both at comfortable walking speed) prediction using only walking speed (speed), derived gait characteristics (excluding 14 speed-correlated features), raw sensor time series, and vGRF. Classification metrics were derived using leave-one-out cross-validation (LOOCV). The single derived gait characteristic speed separates out wearable (digital) insole device knee OA participants versus control subjects.

FIG. 7E show precision-recall curves 750 of the same comparisons in FIG. 7D. FIG. 7F shows a chart 760 of classification accuracy using raw sensor time series data of control subjects versus target knee OA patients using subsets or all 50 sensors at each time-point of the stride (0-100% of the stride). Time-points may start with the stance phase of the right foot and swing phase of the left foot, and end with the swing phase of the right foot and the stance phase of the left foot. Classification accuracy of 1.0 indicates perfect knee OA versus control classification, using data from that time-point.

As shown in FIGS. 7D-7F respectively, XGBoost models are trained and assessed using leave-one-out cross-validation (LOOCV) on vGRF, where models are evaluated by iteratively leaving one subject out, building a model, and evaluating where that subject would be classified compared to the true result (see Methods). This was also performed for derived gait characteristics, and raw sensor time series (flattened strides) data independently. Additionally, walking speed is used as a single variable predictor of target knee OA, at the self-paced comfortable walking speed as both healthy subjects and OA patients walk at such speed. Walking speed alone is able to almost discriminate between target knee OA subjects and healthy controls (auROC=0.981, auPR=0.983). vGRF LOOCV with the wearable insole device also demonstrated high predictive power (auROC=0.967, auPR=0.979). For derived gait characteristics, aspects of gait other than walking speed can be used to predict knee OA. Using derived gait characteristics excluding the 14 characteristics strongly correlated to walking speed, improved classification accuracy (auROC=0.998, auPR=0.993) is observed. Discriminating parameters included Takeoff dynamics, max force (N), Mean COP velocity (mm/s), gait line associated parameters (sd x and sd y of gait line start point (mm), etc. Flattened strides from raw sensor data, which is also independent to walking speed as each stride is interpolated to a consistent one hundred time points, had slightly less accuracy (auROC=0.972, auPR=0.981).

Additionally, the contribution of each sensor at each time point along individual segmented strides to the disease classification accuracy is visualized. Additional XGBoost models on subsets of sensor type at each time point are used, as shown in FIG. 7B. It is determined that classification accuracy for control versus target knee OA depends on the type of sensor, the time point along a stride, and the foot (left versus right).

According to implementations of the disclosed subject matter, subject-specific gait signatures can be individual and consistent across time. Subject-specific gait signatures may be determined in accordance with techniques disclosed herein, as discussed in FIGS. 3A and 3B. According to implementations disclosed herein, models may be trained to identify individual subjects from their walk, or a single stride. Such identification suggests that the gait data collected has data rich enough to identify not just knee disease but potentially other clinical attributes. Such a determination may be made irrespective of disease state, and may focus on identifying the optimal technique to determine an individual participant gait pattern.

Two considerations were observed in generating such signatures. The first is regarding the individuality of human gait patterns. Techniques disclosed herein identify a methodology that is best suited to identify generalizable patterns of any person's gait. The second is determining which methodology captures features of a specific individual's gait that have consistency with time. As discussed herein, training on the same individuals across multiple time points improves the ability of machine learning models to detect features that identify individuals consistently with time.

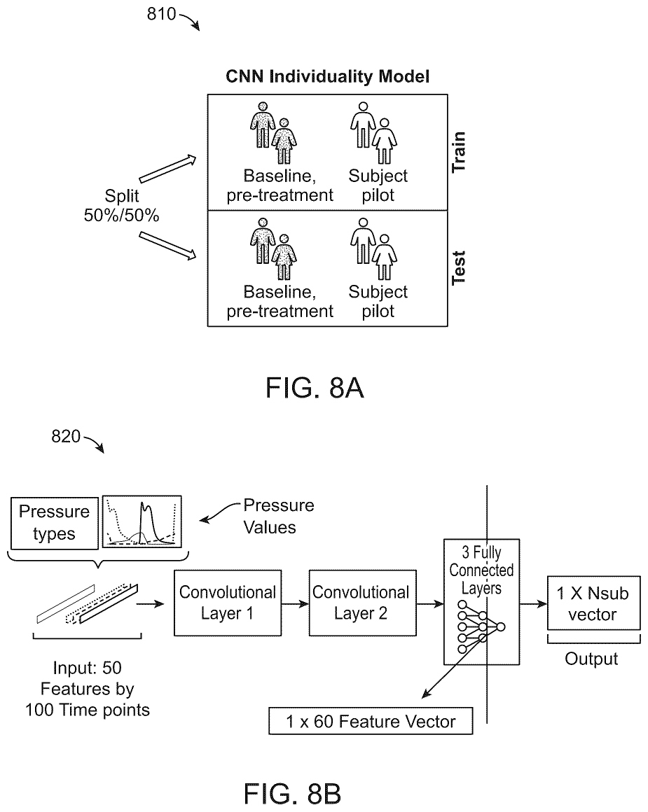

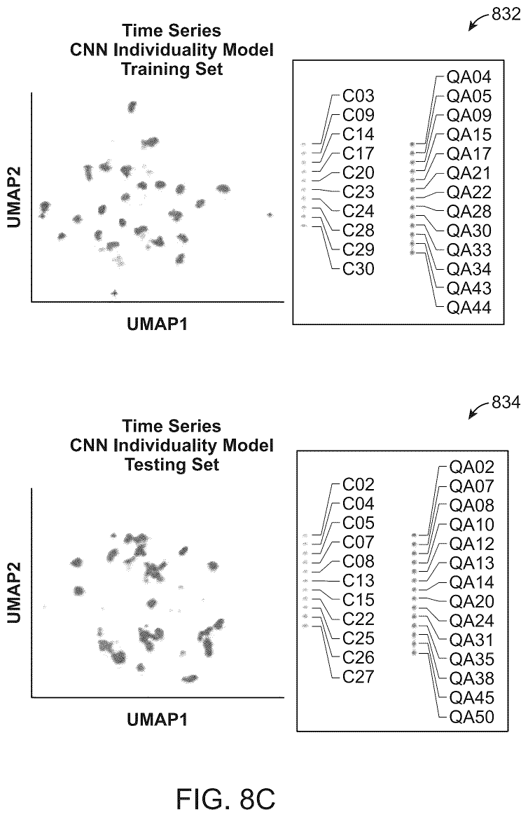

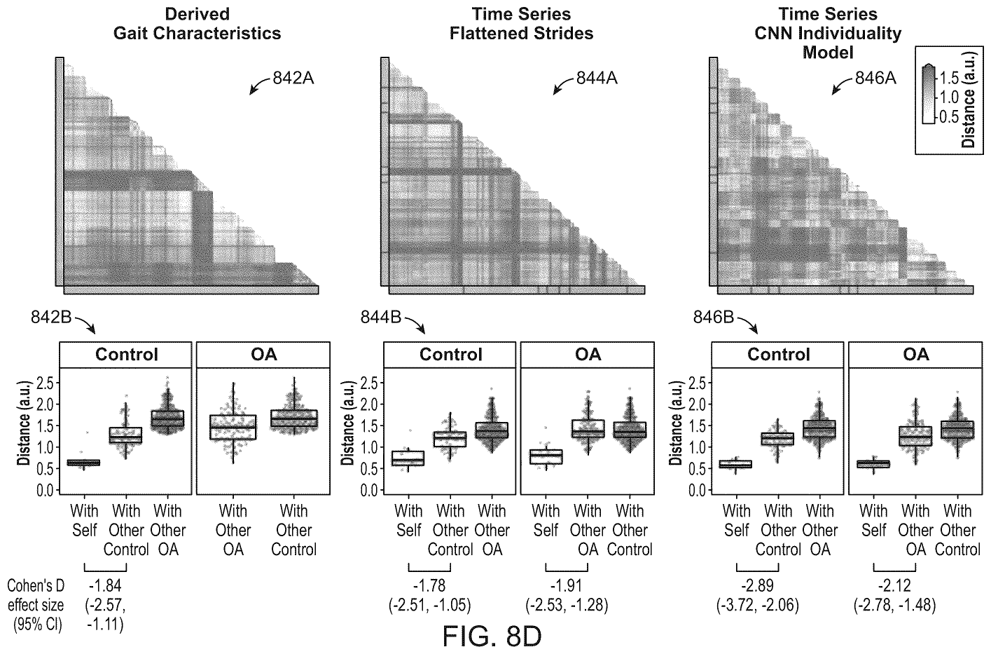

CNN latent representations of raw sensor time series data perform better than derived gait characteristics for deriving individual gait walking signatures, as discussed in reference to FIGS. 8A-8D. FIGS. 8A-8D show that time series analyses of a test wearable insole device data outperform summary parameters for individual gait walking signature derivation. In FIG. 8A, pilot study subjects and knee osteoarthritis (OA) clinical trial patients were split 50:50 into training and testing sets, stratified by disease status, for the first CNN model 810 identifying the individuality of gait patterns.

In FIG. 8B, diagram 820 shows that a CNN was trained on segmented structured strides from the wearable (digital) insole device in the training set, to predict from which subject the stride came. The activation of the last fully connected layer in the CNN includes 60 features and represents the model's latent representation of gait. FIG. 8C shows UMAP clusters 832 and 834 of these 60 latent features for each stride captures the individual identity of participants in not just the training set but also the testing set. Each dot represents a single stride from a subject, color or shade represents each subject, and shapes represent subject's health status (C=control, OA). Intra- and inter-subject clustering and separation is greater in the training set, as expected, and is present in the testing set as well.

FIG. 8D shows spearman correlations/distances in arbitrary units between each pair of walks (for derived gait parameters) or strides (for time series) from the testing set shown as heat maps for each of the three methods (top panels 842A, 844A, and 846A). Subject of the walk/stride are identified along the edge. Boxplots 842B, 844B, and 846B show median spearman correlation and/or mean distance of each walk/stride with other walk/strides from the same individual, and with walk/strides from other individuals separated by disease class (bottom panels of boxplots 842B, 844B, and 846B). Correlations and/or distances are faceted by the disease class of the individual. The more correlated individuals are with other individuals (higher spearman correlation), the more difficult subject-level classification is; a good classifier has high spearman correlation for “with self”, and lower spearman correlation for “with other” classes.

As the test wearable insole devices produced high-frequency raw sensor time-series data, an analyses of whether such structured strides (50 measurements along 100 interpolated time points for each stride) included informative subject-specific gait features is made. To utilize the temporal aspect of the data, a one-dimensional CNN is constructed in which the model interprets the relationship between sequential time points for each sensor. This temporal relationship in the input data was lost in the previous analysis discussed herein, in which the stride was flattened and interpreted by XGBoost.

To determine individuality of walking patterns, as discussed in FIGS. 3A and 3B, subjects were split 50:50 into training and testing sets and stratified by disease status, as shown in FIG. 8A. A 1D-CNN is trained on structured strides of training set individuals. Subsequently, the CNN model is applied on structured strides of testing set individuals. For each stride, the 60 features in the last connected (penultimate) layer of the CNN are extracted, as shown in FIG. 8B. This penultimate layer is the layer before the final output of the CNN model predicting the individual from which the stride came, and thus these 60 features constitute a gait “fingerprint” learned by the CNN model. Accordingly, a CNN trained to learn individual subjects gives greater weights to features of human gait patterns that distinguish individuals. These patterns (captured in the latent features) can subsequently be used for classification of new individuals previously unseen by the model.

To visualize the latent representation of strides from the CNN, UMAP clusters 832 and 834 of FIG. 8C of these latent features from each stride indicate that this representation captured the individuality of participants based on just a single stride. Strides from the same individual clustered together in both the training and testing sets. Models are constructed for each data type to predict individual subjects in the training set, and then applied on the testing set. Next, spearman correlation is evaluated within a subject, within other subjects with the same disease status, and within other subjects with a different disease status, as shown in FIG. 8D.

To quantify the individuality of the latent CNN representation, the spearman correlations of all test set strides in such representation against each other are compared in FIG. 8D. For comparison, spearman correlations of derived gait features are plotted from each walk from each test set subject, and the same strides in raw flattened format. The median correlation between each pair of subjects is computed for each of these three methods, and median correlations are grouped into (1) those within subject, (2) within other subjects with the same disease status, and (3) within other subjects with a different disease status. Strides from the same subject are expected to be greatly correlated, and strides from different subjects to be less correlated.

As shown, the CNN latent representation of the raw sensor time series data performed the best in terms of maintaining high correlation of strides from the same subject and minimizing the correlation of strides from different subjects. Strides from oneself correlated highly across all three methods for both controls and target OA subjects (spearman correlation medians and IQRs for the derived gait characteristics were: controls 0.98 (0.98-0.98); flattened strides: controls 0.83 (0.81-0.85), OA 0.79 (0.76-0.82); CNN latent representation: controls 0.86 (0.81-0.89), OA 0.87 (0.82-0.92)). Using the CNN latent representation results in higher with-self correlations compared to just flattened strides (p=0.0002, paired Wilcoxon signed-rank test).

Using CNN latent representation, correlations are lower when strides from different individuals are compared, comparing strides from two controls: 0.36 (0.24-0.58); from two OAs: 0.51 (0.24-0.58); from one OA and one control: 0.24 (0.05-0.37); for all three categories, p<1E-6 compared to with-self stride correlations, using a Wilcoxon rank sum test.

Correlations of strides from different individuals re also lower using the CNN latent representation versus flattened strides (comparing strides from two controls: p=2E-9; from two OAs: p=0.004; from one OA and one control: p=1E-24, all paired Wilcoxon signed-rank test) and CNN latent representation versus derived gait characteristics (for all three categories, p<1E-10). These results suggest that the CNN latent representations best capture gait individuality, where such representation preserves high correlations amongst strides from the same individual and reduces correlations amongst strides from different individuals.

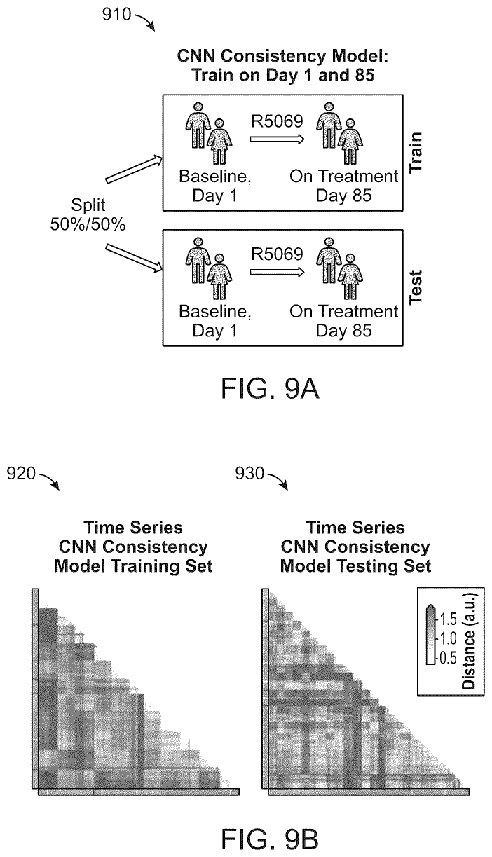

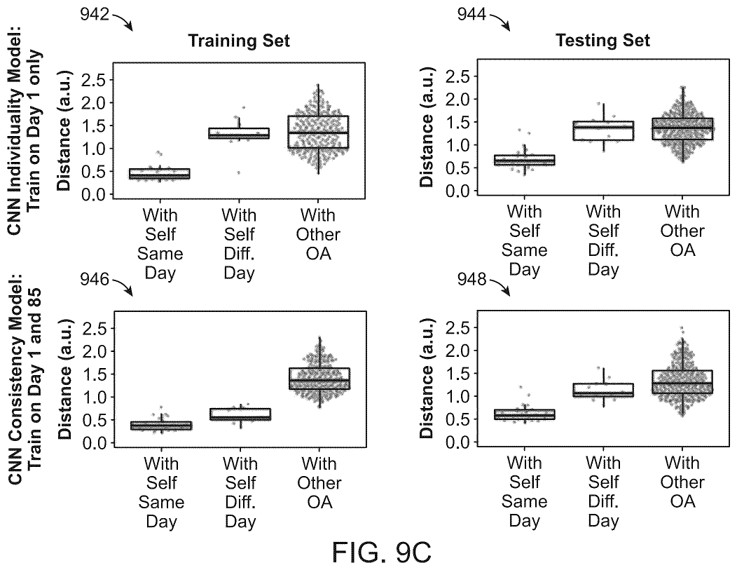

FIGS. 9A-9C are based on training using raw sensor time series data from two days to determine consistency. FIGS. 9A-9C show that training across the two days increases consistency of a CNN model representation of the same participants from different time points.

In FIG. 9A, target OA clinical trial participants are split 50:50 into training and testing sets containing both day 1 (baseline) and day 85 (on treatment) data, for the second CNN model investigating the consistency of gait patterns, as shown in diagram 910.

In FIG. 9B, spearman correlation/distances in arbitrary units between pairs of strides of the latent representation from the second consistency CNN model of each stride in individuals in the training and testing set are shown as heat maps 920 and 930. Subjects of the stride are identified along the edge, with strides from day 1 and day 85 next to each other.

FIG. 9C shows boxplots 942, 944, 946, and 948 of median spearman correlation (or mean distance) of each stride with other strides from the same individual on the same day, from the same individual on a different day, and from other individuals, for both the first individuality model and the second consistency model discussed in FIGS. 8A-8D. Correlations/distances are shown using the different models in both the training and testing sets.

To evaluate whether a model can recognize the strides of participants from different days, test wearable insole device sensor data from both baseline (day 1) and on-treatment time points (day 85) of OA participants in the R5069 clinical trial are shown. A second CNN model is trained on combined data from both time points for training set participants, where input data is labeled only by participant and not by time points. The first model trained only on day 1 data is designated as an “individuality” model, and the second model trained on both days is designated as a “consistency” model, shown in FIG. 9A. For comparability, both models use the same split of train and test participants.

Both models are tested on day 1 and day 85 testing set participants by evaluating the spearman correlation of the CNN penultimate layer of all strides with each other. FIG. 9B plots the spearman correlation for the second consistency model on both the training (left) and testing set (right) participants. For each participant, day 1 and day 85 strides are arranged next to each other, and the cross-day correlations within each participant are shown in squares closest to the diagonal.

FIG. 9C shows stride correlations for within subject from the same day, within subject from different days, and within other subjects. Within the training set, the second consistency model, which learned on both day 1 and day 85 strides, results in higher correlations than the first individuality model, which learned on only day 1 strides, in comparing strides within self from different days (p=0.002, using a paired Wilcoxon signed-rank test). Within the testing set, the second consistency model results in higher correlations than the first individuality model in comparing strides within self from different days (p=0.016, paired Wilcoxon signed-rank test), suggesting that training across multiple days improves the ability of the CNN model to identify features that remain consistent across multiple visits. The results also show additional capacity for model improvement with respect to consistency of gait. In the second consistency model, the correlations within self are higher amongst strides from the same day versus different day in both training and testing participants (p=2E-5 and p=2E-7, respectively, Wilcoxon rank sum test). Collection and training on additional time points beyond two days may result in models that are better able to learn gait features that consistency identify an individual across time.

Accordingly, in FIGS. 4A-9C and the related discussion, digital biomarker data derived from a wearable insole device can be used for detection of disease (e.g., as discussed in relation to FIGS. 1A-2B) and for identifying subject-specific gait patterns (e.g., as discussed in relation to FIGS. 3A-3B). As discussed, test wearable devices can be used to identify clinically meaningful differences between healthy and target disease states (e.g., knee injury or knee OA). Further, linking the appropriate analytical approaches to clinical research determinations, such as the identification of gait signatures, may allow better precision medicine approaches for digital biomarker development. Digital biomarkers with proper analytical considerations can be used at any stage of drug development such as, but not limited to, patient screening, detecting, or monitoring safety, drug dosing, treatment monitoring, and long-term outcome assessments. Test wearable sensors that can replicate and enhance existing production clinical measurements, such as patient-reported outcomes (PROs) in clinical research settings, have can complement existing measures of feeling, function, and survival. An agnostic approach as to the type of data that is optimal to make clinical determinations (a “data first” approach) is disclosed herein, rather than identifying a digital biomarker that exactly replicates a production clinical gold standard.

As discussed herein, a model using a production clinical gold standard is built, and the model is tested on an independent test wearable test set. Additionally, consistent disease versus control differences in vGRF curves are observed by comparing available production force plate data to vGRF generated from a test wearable insole device collected at different times, different places, and in different populations (e.g., target knee injury, OA, and/or controls). As discussed herein, models generated using this data can distinguish target both knee injury subjects and OA subjects from their controls. Accordingly, these vGRF changes may represent true differences between healthy control and target knee arthropathy subjects, allowing wearable insole devices to be used to screen for knee arthropathy and potentially other diseases that impact gait. Although production devices such as force plates may have advantages, the ease and generalizability of using wearable devices in larger populations or trial settings can enable more widespread implementation of these applications. In addition, advantages of a test wearable insole device include addition of more comprehensive gait data such as derived gait characteristics that are shown to improve the detection of a target condition such as a disease. Evaluation of derived gait characteristics from the digital insole indicate that walking speed is in important determinant of knee OA classification, however, when all speed related parameters (e.g., derived gait characteristics highly correlated with speed) are removed from the analysis, a given model is still able to successfully detect test knee OA subjects, highlighting that there are additional features of knee OA gait that differentiate them from controls. Though walking speed is relevant, walking speed may be highly variable and influenced by the setting where measurements are being taken, so a speed-independent approach may allow for a more robust evaluation of gait disease signatures.

As discussed herein, detecting conditions between controls and test condition subjects, where effect sizes are expected to be large, can have outcome implications. Test wearable devices may also successfully detect within-disease disease severity gradations, where effect sizes are smaller, which may identify changes within disease over time or with interventions, and may have utility in clinical practice, and may serve as a useful endpoint tool for clinical trials. As discussed herein, derived gait characteristics make ideal endpoints in clinical research, given that they describe an objective aspect of gait that may have meaning to a health care provider (e.g., total distance walked in meters, or maximum force applied during a 3-minute walk in Newtons).

As discussed herein, raw time series data from test wearable insole devices may be analyzed, and such data from a single stride may be used to identify subjects. Raw time series data that includes subject-specific latent features suggests that multiple clinically-relevant signatures, beyond the one disease signature discussed herein, may be included in the data.

Collecting additional time points from individuals may allow a machine learning model to learn more consistent subject-specific gait patterns. Quantitating individual subject gait patterns may be useful in clinical development for precision medicine applications. Subject-level gait patterns and the ability to identify unique signatures of an individual's gait may allow better monitoring of treatment response over time on a per-subject and on a population-wide level. Training datasets with participant data from multiple visits may improve machine learning model outputs to pull features that remain consistent with time as well as identify parameters that can change over time.

A potential utility of leveraging devices originally-development for other purposes towards clinical research should be appreciated, such as a digital insole developed for athletic sport training. However, the data such devices generate may require clear hypothesis-driven validation to detect relevant signals, similar to research-grade instrumentation. In demonstrating vGRF data from a digital insole may replicate standard clinical data generated from force plates, a criterion validity of vGRF data from digital insoles should be appreciated as digital insoles may replicate the clinical standard (the criterion) at least to a degree. An external validity within a digital insole study of disease gait signature across both methodologies (e.g., force plate and digital insole) using a machine-learning approach, with a training set built on force plate data and evaluated on force plate and digital insole data collected elsewhere, should be appreciated. Analytical strategies may maximize a clinical understanding and a generalizability to other studies, and an analytical approach maximizes construct validity (i.e., how close a digital biomarker reads out the “construct” it is intended to measure). In some instances, maximization of construct validity may influence face validity (i.e., a degree to which a measure is intuitively interpretable). In this instance, as raw sensor data lacks face value interpretability and improves an ability to determine subject-specific gait patterns, digital biomarker data containing disease or subject specific information that may be leveraged in alternative clinical circumstances may be determined.

Production force plate and test wearable sensor data may be harmonized. Though vGRF values may be objective and platform independent, the vGRF from a test wearable insole device may be off-scale relative to a production gold standard force plate. Such off-scale data may be used for wearable device validation, though may be limited based on small sample size and/or subjects not being demographically and clinically matched with the OA study. As disused herein, positive classification performance for control subjects relative to their production force plate counterparts can be determined. It should be appreciated that although the examples shown and described herein are generally directed at knee arthropathies, the systems and methods may be similarly applicable to various other diseases and/or injuries that are affected by an individual subject's gait without departing from a scope of this disclosure. Additionally, a machine learning modeling approach to approach computation of individual gait consistency is disclosed herein which may be improved with additional time point data. Derived gait characteristics and raw sensor time series data are not limited to test wearable devices disclosed herein and may be outputs of production force plate data as well. As discussed herein, production force plate and test wearable device data are generated “out of the box”, where derived gait characteristic level data may not be available for a production force plate dataset. Time series data for production force plates may be limited to a two-dimensional force distribution captured over the course of 1-2 steps (seconds of data), and, thus, may be different in nature compared to test wearable device digital sensor raw sensor time series data collected over longer spans of time. It will be understood that techniques disclosed herein may be applied to other types of devices and/or data. For example, a gaming console balance board could be validated against gold standard force plates to measure balance.

The techniques disclosed herein provide a framework for an integrated analysis of test wearable insole sensor data for use in digital endpoint development. To identify disease signatures, a machine learning model may be built using data from production force plates, a clinical gold standard. The use data may be derived from a test wearable device and show comparable disease classification with external datasets. Analysis of types of test wearable data is treated in an agnostic way to show that there is no “one size fits all” test wearable data pipeline. The techniques disclosed herein may further provide an understanding of an influence of a therapeutic intervention for an individual, such as auditing in accurate diagnosis, longitudinal monitoring of disease progression, or response to treatment.

This example demonstrates that a cluster of parameter, including, for example, 14 derived gait characteristics, consistently correlated with walking speed using a conservative cutoff (e.g., |ρ|>0.7). Moreover, using principal component analysis (PCA) dimensionality reduction, a continuum linking the arthropathy state of knee OA and walking speed is observed. Subjects with knee OA generally exhibited a slower walking pace.

In this example, analysis is not limited to only classifying ‘slow’ versus ‘normal’ walking. A broader range of gait characteristics are analyzed to ascertain if these features could enhance the accuracy of classifying knee OA relative to control, beyond the factor of speed alone. Derived gait characteristics that excluded the 14 speed-correlated attributes are used to achieve higher classification accuracy (auROC=0.998, auPR=0.993) than speed alone. These results may suggest that a deeper understanding of gait can be captured with wearable devices (e.g., digital insoles), beyond speed, to detect disease-specific features.

According to an implementation, a model disclosed herein may be compared with a simpler model trained on walking speed. According to this example, it is determined that walking speed (e.g., walking speed, alone) may distinguish between knee OA subjects and healthy controls with substantial accuracy (auROC=0.981, auPR=0.983).

### Example 2

In accordance with a second example conducted in accordance with the techniques disclosed herein, associations between derived gait characteristics, captured by a digital insole, and imaging (K-L score) or Performance Outcome Assessments (PROs) such as Western Ontario and McMaster Universities Osteoarthritis (WOMAC) metrics are determined. According to this second example, interrelation between laterally derived gait characteristics (e.g., derived separately for the Left or Right insoles) and two laterally collected clinical measures: the WOMAC pain sub-score and the K-L imaging disease severity score, are observed. The relationships are independently evaluated for each joint.

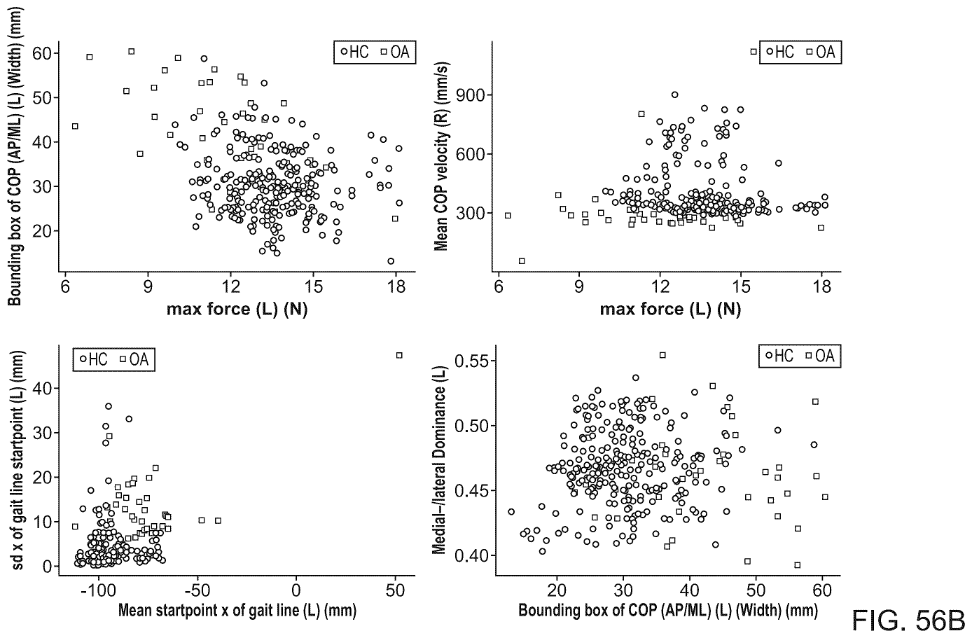

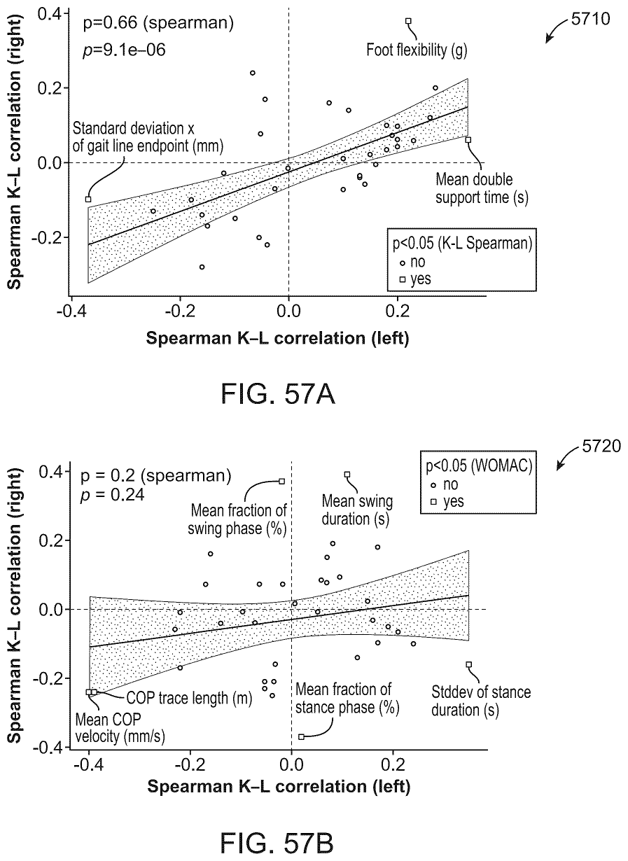

Each derived gait characteristic is correlated from the respective joint (either left or right) individually with the corresponding joint's 1) WOMAC pain sub-score or 2) K-L score. Some of the correlations/traits demonstrated nominal significance for at least one join. FIGS. 57A-57B show example Spearman K-L correlations as a factor of the left joint and right joint. Chart 5710 of FIG. 57A shows such K-L correlations with a p equal to 0.66 (spearman) and chart 5720 of FIG. 57B shows such K-L correlations with a p equal to 0.24 (spearman). The distribution of nominal spearman rho values between joints allows for analysis of these relationships when viewed as a scatter plot, for example, to assess if the relationship is similar between joints. For example, if these correlations are similar for each joint, they may represent a trend (e.g., a real trend).

Example 2 is conducted using an N of 40 subjects, with only an N of 14 subjects having knee-only OA. Example 2 can further be supplemented to determine whether different joints act independently. For example, Example 2 may further be supplemented by determining a subject's dominant foot (e.g., left vs right footed) and/or controlling for pain on only a single joint (e.g., a KL score of 0 (zero) on at least one joint).

### Example Materials and Methods

The example implementation disclosed herein may be used to characterize data from a wearable insole device, demonstrate its utility relative to a production clinical gold standard, and to determine optimal analytical techniques and data types for the analysis relevant to clinical implementations. The materials and methods to implement the example are further disclosed herein.

Three datasets were integrated for analysis in the example. GaitRec force plate vGRF datasets for force plate control subjects (N=211) and knee injury subjects (N=625) were included. In other words, the GaitRec force plate dataset (force plate data) contains N=211 healthy controls, who walked at three different walking speeds (slow, comfortable, and fast), and N=625 knee injury subjects, who walked at a comfortable walking speed. A dataset of healthy control participants (N=22) from a pilot study of a digital insole conducted between July 2019 and August 2019, described in Table 2, was also included. In other words, a second dataset may be from a digital insole pilot study, where N=22 healthy controls walked at three different walking speeds (slow, comfortable, and fast). A third dataset may be from a digital insole sub-study, such as from a longitudinal clinical trial in knee osteoarthritis (OA), where N=40 knee OA subjects performed a 3-minute walk test (3MWT) at a comfortable walking speed at baseline (pre-treatment) and at day 85 (on treatment). Both force plates and digital insoles may produce data that is collected during stance and swing phases of a person's gait cycle. The types of data that may be produced by these devices may include vertical ground reaction force (vGRF), derived gait characteristics, and/or raw sensor time series. In this instance, a derivation of gait disease signatures of knee OA, and/or an individuality and consistency of gait patterns may be determined. Two analytical methods may be used to evaluate this data, XGBoost, a gradient boosting classifier, may be used to analyze vGRF, derived gait characteristics, and raw sensor time series flattened stride data. A one-dimensional convolutional neural network (CNN) may be used to analyze structured stride raw sensor time series data.

The date of first enrollment in the pilot study was Jul. 6, 2019 and last participant visit was Aug. 5, 2019. Study candidates who were pregnant or had a body mass index (BMI) above 40 kg/cm2 were excluded from the study. All participants were recruited internally within the Regeneron facility located in Tarrytown, NY, USA. All participants provided written informed consent prior to participation in this study, and this study is exempted research under the Common Rule (45 CFR Sec 46.104). As part of a clinical trial evaluating the impact of a novel pain therapeutic in moderate to severe knee OA (NCT03956550), a sub-study for a digital insole device was performed to collect data for gait assessment in knee OA patients. All patients in this sub-study were enrolled at two study sites in the USA and Moldova and the study was conducted between June 2019 and October 2020. The date of first enrollment in the R5069-OA-1849 trial was Jun. 17, 2019, and last patient visit was Oct. 29, 2020. The sub-study targeted to enroll approximately 13 patients per treatment group to obtain data on at least 10 patients per treatment group for a total of approximately 30 patients across the entire sub-study. Eligible participants were men and women who were at least 40 years of age at the time of study entry with a clinical diagnosis of OA of the knee based on the American College of Rheumatology criteria with radiologic evidence of OA (Kellgren-Lawrence (K-L) score≥2) at the index knee joint as well as pain score of >4 in Western Ontario and McMaster Universities Osteoarthritis Index (WOMAC) pain sub-scale score. The WOMAC score is a self-administered questionnaire consisting of 24 items divided into 3 subscales, where the pain sub-score is assessed during walking, using stairs, in bed, sitting or lying, and standing upright. The study protocol received Institutional Review Board (IRB) and ethics committee approvals from Moldova Medicines and Medical Device Agency and National Ethics Committee for Moldova, and the Western Institutional Review Board (WIRB).

Table 3 shows the dates of first and last enrollments of subjects in the pilot study and clinical trial:

TABLE 3

| | Date of first | Date of last |
|---|---|---|
| Cohort | enrollment | patient last visit |
| Pilot study | Jul. 6, 2019 | Aug. 5, 2019 |
| R5069-OA-1849 trial | Jun. 17, 2019 | Oct. 29, 2020 |
| (NCT03956550) | | |

Table 4 shows baseline characteristics and gait assessments of subjects in the digital insole pilot study and patients with knee OA in the R5069-OA-1849 clinical trial (digital insole sub-study). It should be appreciated that references to “K-L” in the table below refer to Kellgren-Lawrence:

TABLE 4

| | | | | Clinical trial |
|---|---|---|---|---|
| | | | Pilot study | (N = 44 enrolled |
| | | | (N = 22) | in sub-study, N = 43 |
| | | Knee | Cross | data collected) |
| | Control | injury | sectional | Longitudinal |
| Age | | | Mean: 39 | Mean: 62.75 years |
| | | | years | Median: 63 years |
| | | | Median: 35 | Range: 52-77 years |
| | | | years | |
| | | | Range: 19- | |
| | | | 85 years | |
| Sex | | | 11 Female | 28 Female |
| | | | 11 Male | 8 Male |
| BMI (kg/m²) | | | Mean: 26.2 | Mean: 34.5 |
| | | | Median: 26.0 | Median: 34.8 |
| | | | Range: 20.1- | Range: 26.6-38.9 |
| | | | 37.0 | |
| Arthropathy | | | N/A | K-L2-3: N = 23 |
| class | | | | K-L4: N = 13 |
| Walk test | | | Walk straight | 3-minute walk test |
| | | | for 30 | |
| | | | seconds | |
| Walking speed | | | 3 speeds | 1 speed (comfortable) |
| | | | (comfortable, | |
| | | | fast, slow) | |
| Number of walk | | | ~12 times (at | 1 time at baseline and |
| test performed | | | each speed) | 1-time on-treatment |
| | | | | (day 85) |
| Total length of | | | 20 minutes | 3 minutes |
| gait evaluation | | | | |

In the pilot study, each study participant walked straight along a hallway with a hard tile floor at three different qualitative speeds for ˜12 times at each speed (about 36 walks total for each participant). For each walking trial, participants wore the wearable insole inside their own shoes and were prompted to walk at a normal or comfortable speed, walk fast as if they were in a hurry (fast speed) or walk slow as if they were at leisure (slow speed). Prior to the walking trials, each participant was instructed to practice walking around to get accustomed to the insole. Participants' clinical and demographic information were also collected prior to walking trials.

In the R5069-OA-1849 clinical trial, a total of N=44 OA patients were enrolled into a sub-study of a 259-patient clinical trial. Patients were required to bring the same pair of shoes to the study site to perform a 3-minute walk test with the wearable insole. Each patient performed the task twice, once at baseline and the other 85 days later post-treatment.

To normalize the vGRF data across production and test devices and subjects due to the differing sampling frequencies of force plates and wearable insole, smoothing spline functions (scipy.interpolate.interpld) were fit to vGRF time series sensor data from both GaitRec force plate data and wearable insole computed vGRF data. vGRF curves were bounded by 0, and 100 evenly spaced time points across the curve were derived for each curve (to derive a % stance phase). All vGRF curves were normalized by participants body weight in Newtons. Within each device, the vGRF curves were further normalized using a z-transformation within each stance phase time point, within each device (e.g., as shown in FIG. 5A).

Wearable insole raw sensor time series data processing is further discussed herein. The wearable insole collects 25 100-Hz measurements for each foot (50 measurements across both feet), including 16 measurements from 16 vertical plantar pressure sensors, 3 x,y,z measurements from an accelerometer, 3 x,y,z measurements from a gyroscope, 1 measurement of total force, and 2 x %,y % measurements of center-of-pressure. This raw sensor time series sensor data for both the R5069-OA-1849 clinical study and pilot study was preprocessed with custom scripts written in Python 3.6.

For the following analysis, a “walk” is defined as data captured by the wearable insole while the subject completed the researcher's walking task (typical duration of 180 seconds for the R5069-OA-1849 clinical study and 25 seconds for the pilot study). A “stride” is defined as the data captured by the wearable insole between the peak pressure of the right heel (the average of the wearable insole right pressure 1 and 2 sensors) and the next peak pressure of the right heel. A typical stride duration is 1-2 seconds, highly dependent on individual walking speed.

Data preprocessing for each subject was performed separately. First, each walk was segmented into individual strides. Since the wearable insole did not collect data in regular intervals, each stride was then interpolated for each of the 50 sensors to obtain 100 time points along the stride. Thus, each interpolated stride consists of 50 vectors (one for each sensor), and each vector is 100 units long.

For the pilot study, walk from each subject was processed individually, treating slow, comfortable, and fast walks separately. Walks without all 50 features and walks with greater than 1% missing data were excluded. For the remaining walks, any missing data was linearly interpolated (scipy.interpolate.interpld).

Each walk was then segmented into strides, and each stride was interpolated to 100 time points. To segment a walk, peaks were identified in the average time series of the wearable insole right pressure sensors 1 and 2, located in the right heel using scipy.signal.find_peaks with parameters width=10 and prominence=5. The walk was segmented using the peaks, and the number of measurements in each segment was calculated. Segments that had that an outlier number of samples was an (outliers defined as 1.5*iqr+/−q3 or q1) were excluded, such that only regularly repeating segments, or strides, were analyzed. Each of the 50 features in each stride was then linearly interpolated (scipy.interpolate.interpld) to 100 time points. Only walks with at least 10 interpolated strides were further analyzed.

Under the assumption that an individual's strides within a walk should be highly regular to each other, each stride's Pearson r correlation with the mean of the remaining strides was computed (stats.pearsonr), and any strides with an outlier Pearson r correlation (outliers defined as 1.5*iqr+/−q3 or q1) was excluded. The process was then repeated with remaining strides, to obtain a list of the Pearson r coefficients of each stride with the means of the other strides. The entire walk was excluded if the mean of the Pearson r coefficients fell below 0.95. This procedure was repeated one last time, across all walks by an individual at the same walking speed (slow, comfortable fast). That is, each stride's Pearson r correlation with the average of remaining strides in all walks at the same speed was computed. Again, assuming strides within a subject and within a given walking speed should be consistent with each other, strides with an outlier Pearson r correlation were excluded (outliers defined as 1.5*iqr+/−q3 or q1). Lastly, features dependent on body weight (i.e. pressure sensors and force sensors) were normalized by the subject's mass.

For the R5069-OA-1849 clinical study, data was processed similarly with the following exceptions. These OA patients had only two walks, on one day 1 and one on day 85, which were processed separately. Walks with greater than 5% missing data were excluded, and an entire walk was excluded if the average Pearson r correlation fell below a Pearson r coefficient of 0.90.

Derived gait characteristics and identification of speed-correlated data is discussed herein. The wearable insole derives 85 gait parameters from each walk. Of those, 3 are directly related to the length of the walk (walking distance and left and right center-of-pressure (COP) trace length) and were excluded from further analysis, leaving 82 derived gait characteristics.

Spearman correlations between these 82 parameters were calculated across all walking speeds (slow, comfortable, fast). The silhouette method was used to determine the optimal number of clusters with the factoextra package in R with function fviz_nbclust with 100 bootstrapped samples. To understand aspects of gait other than walking speed, all parameters against were correlated against walking speed, and conservatively 14 parameters that may be influenced by walking speed in any way were removed (|Spearman rho|>0.7). This allowed for an investigation into gait parameters less influenced by of walking speed.

A UMAP technique for dimensionality reduction was applied using the R UMAP package with default parameters to the z-transformed vGRF data from both the force plate and wearable insole device datasets to investigate batch effects.

Principal component analysis of the wearable insole vGRF, derived gait characteristics, and raw sensor time series was performed using the prcomp function in the R stats package. Heat maps of the wearable insole parameters are displayed per individual, averaged across all individual walks. All heat maps display derived gait characteristics after z-transformation by row across all subjects. All clustering on heat maps is unsupervised within groups.

For machine learning model building, XGBoost models were built using vGRF, derived gait characteristics, and raw sensor time series processed data using the sklearn and xgboost packages in Python.

The force plate dataset was randomly split into 85% training and 15% hold-out test datasets. The 85% training data was used for leave-one-out cross-validation (LOOCV) and to construct a final trained model. This final trained model was then evaluated on the hold-out test dataset. The wearable insole device dataset was also used as independent dataset to evaluate the model against.

Leave-one-out cross-validation (LOOCV) may be used to compare XGBoost models trained on the different data types collected by the wearable insole device, including the vGRF data, derived gait characteristics, and raw sensor time series.

Model performance was evaluated using multiple methods. Receiver operating characteristic (ROC) and precision-recall (PR) curves were used to evaluate overall performance. Additionally, the area under the receiver operating characteristics (auROC) curve was quantitated, which describes model performance regardless of baseline likelihood for either class. In addition, the area under the precision-recall curve (auPR) and F1-scores were quantitated, which are useful for evaluating datasets with class imbalances.

Subject-specific gait signatures were determined, as discussed herein. Models were trained to identify individual subjects from their walk, or from just a single stride, suggesting that the gait data collected has a minimum ability to identify attributes (e.g., beyond knee disease). Gait signatures were identified irrespective of disease state, to identify the optimal method to determine an individual participant's gait pattern.

Two considerations regarding clinical research settings were made. The first is regarding the individuality of human gait patterns. Techniques disclosed herein were implemented to determine which methodology is best suited to identify generalizable patterns of any person's gait. The second consideration is determining which methodology captures features of a specific individual's gait that have consistency with time. Techniques were implemented to determine whether training on the same individuals across multiple timepoints improves the ability of our models to detect features that identify individuals consistently with time.

A CNN model for control verses OA classification was applied. For the control versus OA model, model performance was determined using leave-one-out cross-validation. A CNN was trained using all strides from all participants except from one left-out participant, after which the model was evaluated on all strides of that left-out participant. Each participant was used as a left-out test participant in one model, such that for N participants, there were N different CNN models each trained on the other N−1 participants. Each stride was labeled as to whether it came from a control or an OA participant.

For each CNN model, strides from the N−1 training participants were split into an 80% training set and a 20% validation set. Each feature in within each stride was scaled into 0-min to 1-max range across the 100 interpolated time points. The normalized data from each stride was then used as the input to the following CNN architecture: (Functions from the Python [v3.9.7] PyTorch [v1.8.0.post3] package torch.nn were used with the default parameters unless otherwise noted.)
First 1D convolution layer with 50 in channels, 64 out channels, and a convoluting kernel of size 3 (ConvId).
Element-wise rectified linear activation unit (relu in torch.nn.functional).
1D max pooling with a sliding window kernel of size 2 (MaxPoolId).
Dropout with 0.2 probability (Dropout).
Second 1D convolution layer with 64 in channels, 128 out channels, and a convoluting kernel of size 3.
Element-wise rectified linear activation unit.
1D max pooling with a sliding window kernel of size 2.
Dropout with 0.2 probability.
Flatten data to a linear vector of 2944 elements.
First fully connected layer with 2944 in channels and 120 out channels (Linear).
Element-wise rectified linear activation unit.
Dropout with 0.2 probability.
Second fully connected layer with 120 in channels and 32 out channels.
Element-wise rectified linear activation unit.
Dropout with 0.2 probability.
Third fully connected layer with 32 in channels and 1 out channel.
Logistic sigmoid function (sigmoid in torch).

Binary cross entropy loss (BCELoss) used as the loss function, and stochastic gradient decent (SGD in torch.optim) with a learning rate of 0.001 and momentum of 0.9 was used as the optimizer. Data was loaded into the CNN in batches of 32 with shuffling (DataLoader in torch.utils.data), and backwards propagation and parameter optimization were conducted in such batches. Models were trained for 10 epochs, and model parameters from the epoch with the best accuracy on the validation set were chosen as the final model parameters. The model was then tested on the strides of the left-out participant. Model predictions, for whether each stride from the left-out participant was from a control or an OA participant, were aggregated across the N CNN models, and the overall classification performance was computed.

A CNN model for subject classification and latent representation is disclosed herein. As the wearable insoles produced high-frequency raw sensor time-series data, a determination was made regarding whether such structured strides (e.g., 50 measurements along 100 interpolated timepoints for each stride) included informative subject-specific gait features. To utilize the temporal aspect of the data, a one-dimensional CNN in which the model could interpret the relationship between sequential timepoints for each sensor was utilized. This temporal relationship in the input data not included in the previous analysis, in which the stride was flattened and interpreted by XGBoost.

For the individuality and consistency CNN models, the model was trained to identify the subject from which a stride came. However, the purpose of using the CNN model was not classify training subjects based on their strides, but rather to extract activation of the penultimate fully connected layer for the model's latent representation of the “gait fingerprint” of a stride. As such, once the CNN model was trained on participants in the training set, the model was then applied to participants in the hold-out testing set and latent representations for each stride were extracted.

To train the CNN model, strides from the training participants were split into an 64% training set, a 16% validation set, and a 20% final validation set. A similar CNN architecture was used as before, except now rather than a binarized control versus OA output, the model outputs the subject label. As such, the model architecture differed starting from the second fully connected layer:

Second fully connected layer with 120 in channels and 60 out channels.

Element-wise rectified linear activation unit.

Dropout with 0.2 probability.

Third fully connected layer with 60 in channels and 23 out channels.

The CNN model was trained in the same manner as before, except multi-class cross entropy loss (CrossEntropyLoss) was used as the loss function. As before, the model was trained for 10 epochs, and model parameters from the epoch with the best accuracy on the validation set were chosen as the final model parameters. The final validation set was then used to check the final model's performance. A forward hook (register forward hook in torch.nn.modules) was attached to the penultimate fully connected layer, to extract activation of that 60 element layer for a new stride inputted into the model.

Evaluation of subject individuality and consistency in different models is disclosed herein. Models were constructed for each data type to predict individual subjects in the training set, and then applied on the testing set. Next, distances between each pair of walks/strides were calculated within a subject, within other subjects with the same disease status, and within other subjects with a different disease status.

Each feature was first z-scored (centered and scaled to unit variance, using scale function in base R), and Euclidean distances between all walks/strides in the testing set were calculated using dist function in the R stats package. To compare across representations with differing number of features, distances were divided by the square root of the number of features. The mean distance between every two participants (including with oneself) was then calculated.

To evaluate models for subject individuality, each participant-to-participant comparison was categorized into the groups of control within-self, OA within-self, control with another control, OA with another OA, or one control with one OA. Significance of difference in distances between participant categories was analyzed with t-tests in the R stats package. Effect sizes as Cohen's D were computed with the R effsize package.

Evaluation of CNN models of subject individuality and consistency is disclosed herein. Wearable device data from both baseline (day 1) and on-treatment timepoints (day 85) of OA participants in the R5069-OA-1849 clinical trial was used to evaluate whether training on data from two days instead of just one day improves the consistency of the CNN representation of participants. A second consistency CNN model was trained on combined data from both timepoints for training set participants, where input data was labeled only by participant identity and not by timepoint. For comparability, both the individuality and consistency CNN models used the same split of train and test participants. Both models were given day 1 and day 85 of testing set participants, and the distance between all stride pairs as represented by the penultimate CNN layer was calculated as before.

To evaluate models for consistency, each participant-to-participant comparison was then categorized into the groups of within-self same day, within-self different day, or subject with another subject. Only OA participants were analyzed for consistency as only they were assessed on two different days. Significance of difference in distances between the CNN individuality and consistency models across the same participant comparisons was analyzed with paired test-tests in the R stats package.

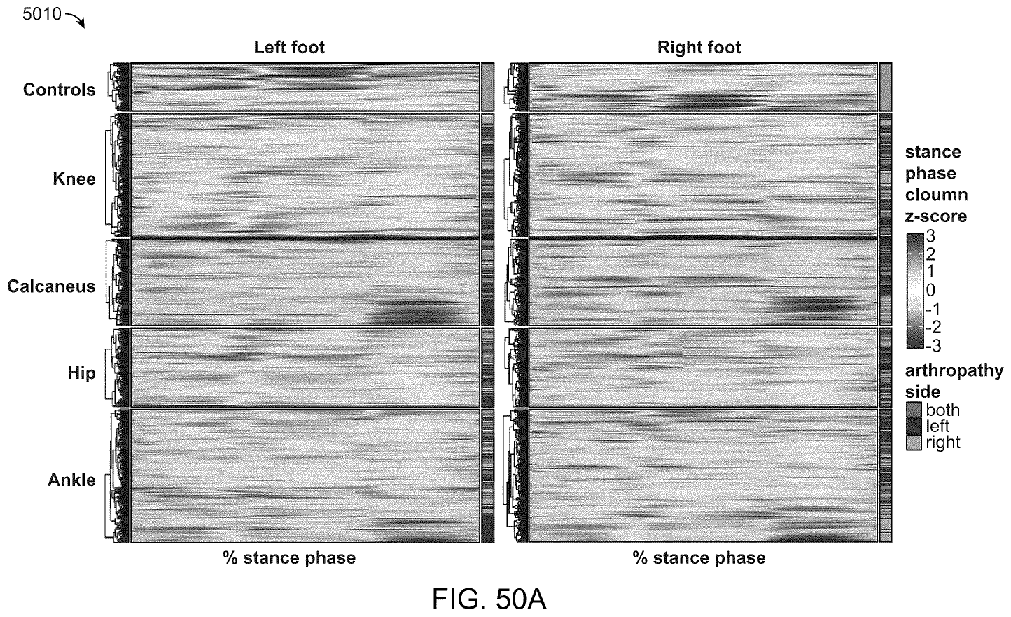

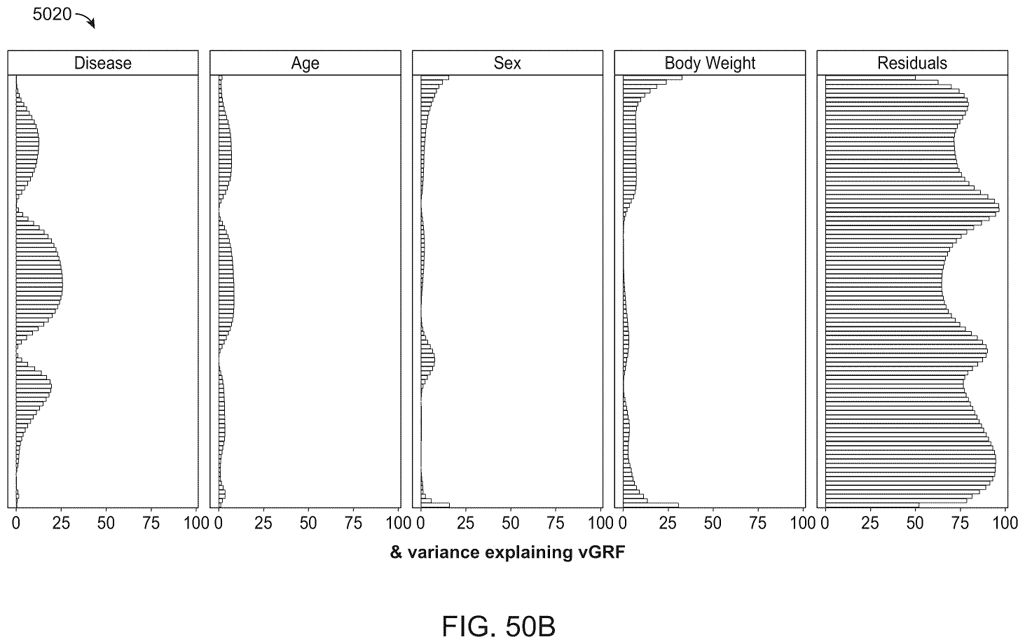

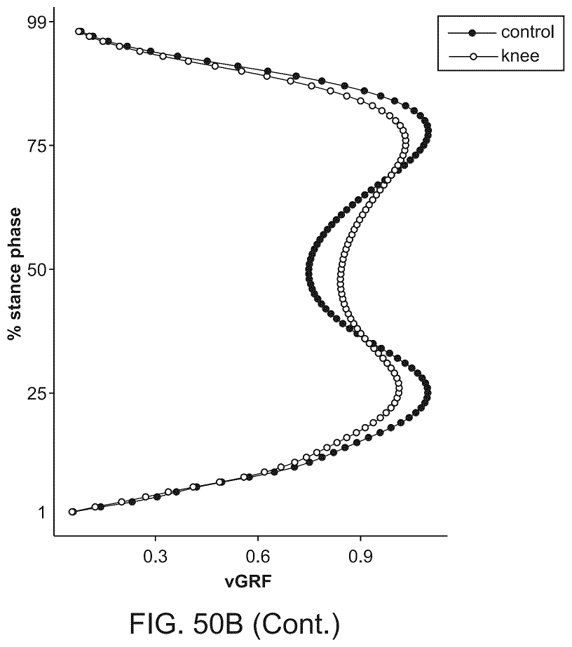

In accordance with the implementations disclosed herein, FIG. 50A shows a heatmap representation 5010 of vGRF data from GaitRec dataset for all joints with injuries and controls. Data are z-scored by each column (% stance phase) across all walks. Heatmaps are separate by injury class (control, knee, calcaneus, hip, and ankle), and vGRF from each walk are unsupervised clustered within each category. The right of the heatmap annotates the joint side with the arthropathy (left joint, right joint, both joints, or no injury in the control group).

In accordance with the implementations disclosed herein, FIG. 50B may depict a variance in vertical ground reaction force (vGRF) with clinical and demographic characteristics of participants. Specifically, FIG. 50B shows linear models 5020 fit at each percent stance phase (timepoint), excluding the edges of the curve which are bounded by zero, and as such have no variance. Disease (e.g., knee arthropathy, or control), age, sex (e.g., male or female), and body weight may be designated as covariates in the model, with each subsequent vGRF percent stance phase timepoint being designated as the dependent variable. Within each linear model, at least partially based on a sum of square for each category compared to a total sum of squares, a variance of each component's contribution to the total variance may be determined, with the residuals indicating an unexplained variance in the models. In some examples, a disease state may be determined to be a substantial contributor to vGRF for most of the curve, with age, sex, and body weight contributing to a relatively smaller proportion of the variance.

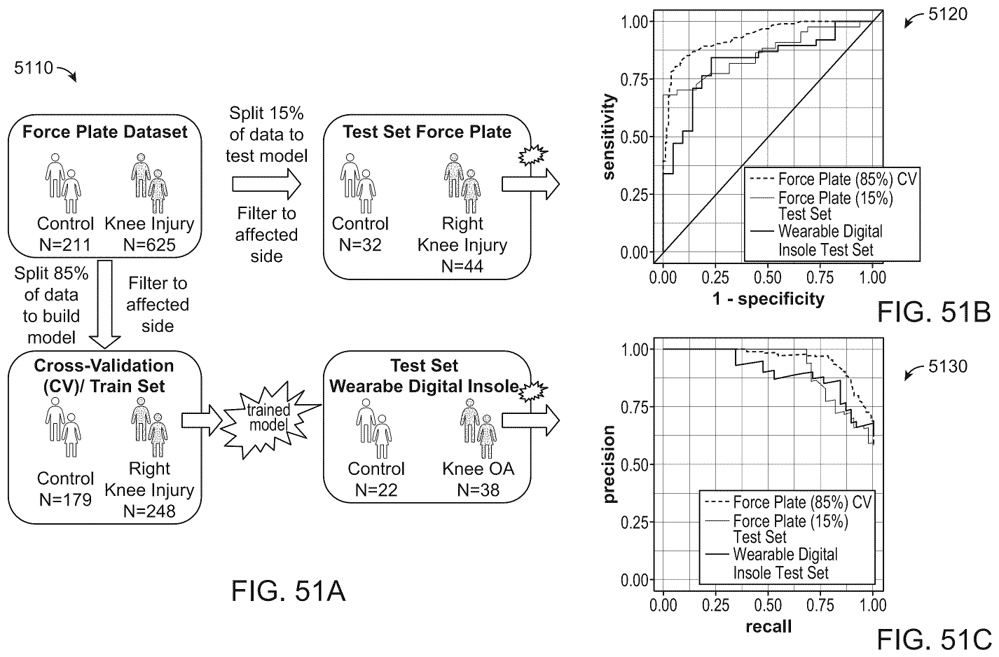

In accordance with the implementations disclosed herein, FIG. 51A shows a schematic of ML (machine learning) model 5110 building of training/validation and testing sets with the right foot data, as discussed herein. FIG. 51B shows an ROC (receiving operating characteristic) curve 5120 for XGBoost classification of force plate (85%) cross-validation (CV, training/validation) set, force plate (15%) hold-out test set, and the digital insole test set for right foot data. FIG. 51C shows a precision-recall curve 5130 for XGBoost classification of the same groups in B for right foot data.

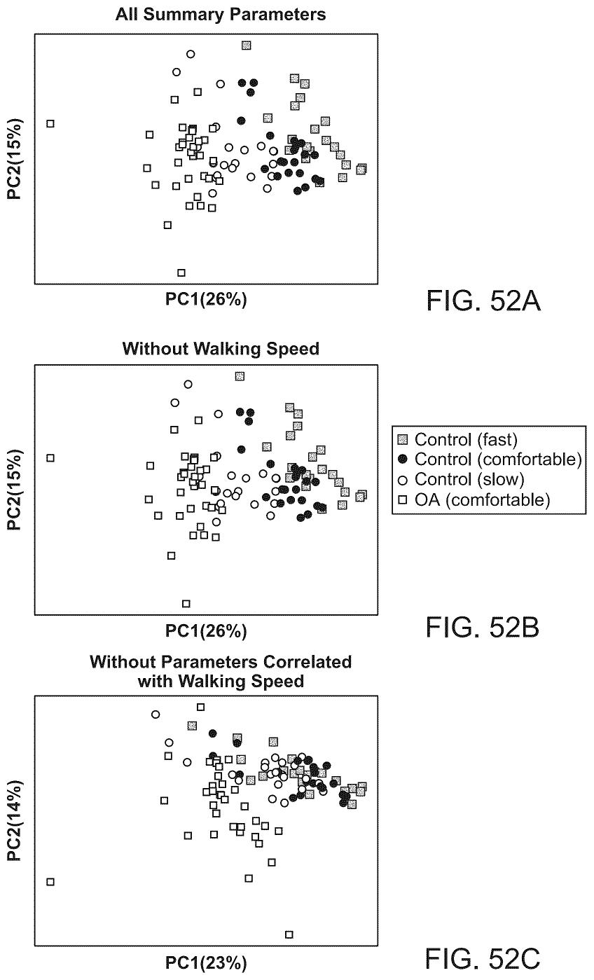

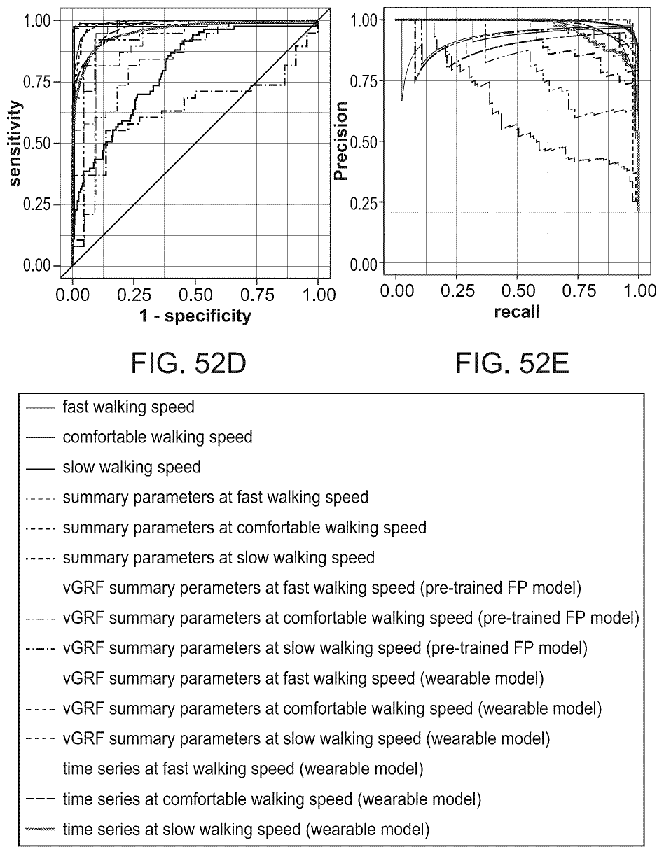

In accordance with the implementations disclosed herein, FIG. 52A shows a PCA (principal component analysis) of all derived gait characteristics measured using the digital insole device, where each point represents the average of all walks from a particular subject, and the dot shade indicates the group (control or knee osteoarthritis OA) or walking speed of control subjects. FIG. 52B shows a PCA analysis as in FIG. 52A, without the walking speed gait characteristic. FIG. 52C shows a PCA analysis as in FIG. 52A, without the 14 derived gait characteristics correlated to walking speed. FIGS. 52D-52E show classification performance auROC using walking speed as a sole predictor, vertical ground reaction force (vGRF) data, derived gait characteristics, and time series data.

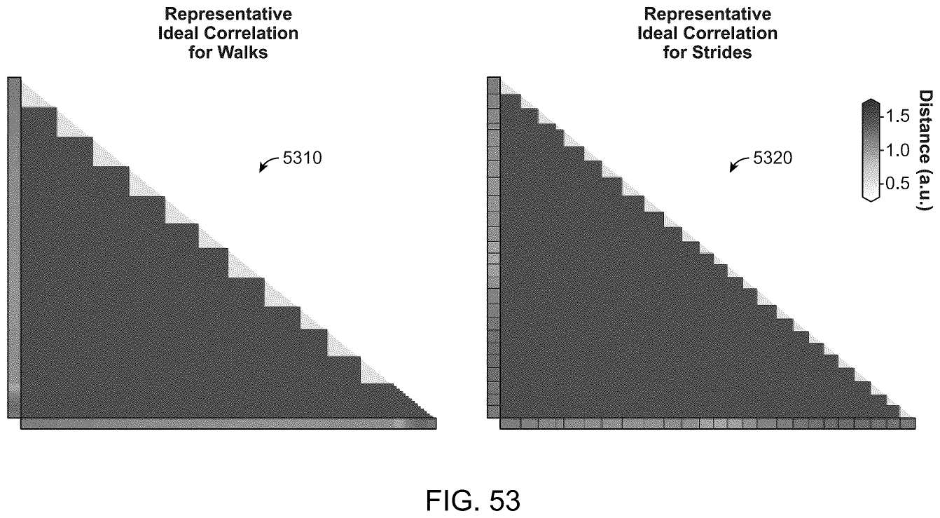

In accordance with the implementations disclosed herein, FIG. 53 shows example heatmaps 5310 and 5320 of a good representation to quantitate individuality and that has low distance between all pairs of walks/strides from the same participant and high distance between all pairs of walks/strides from different participants. Data along the edge indicates each person.

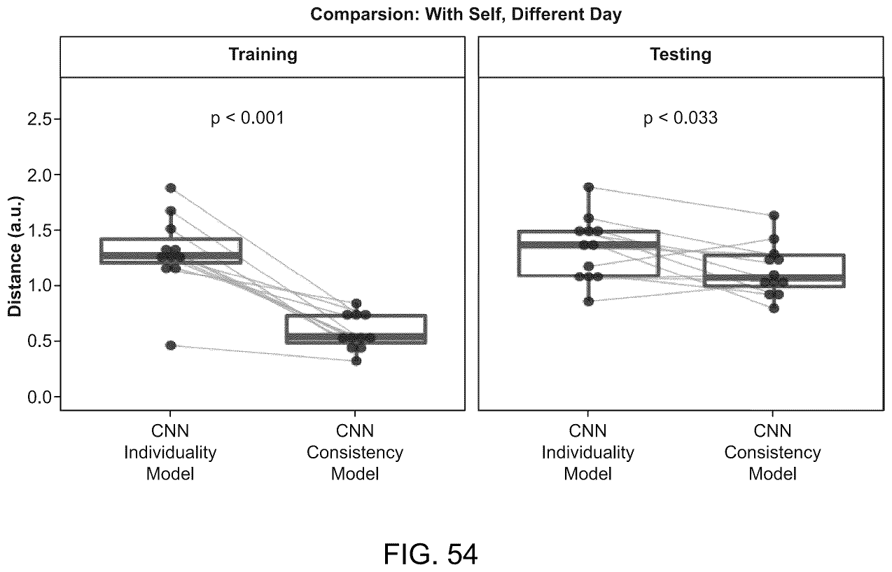

In accordance with the implementations disclosed herein, FIG. 54 shows boxplots 5410 of mean distance (in arbitrary units) of each stride with other strides from the same person on different days, for both the convolutional neural network (CNN) individuality model and CNN consistency model (FIGS. 9A-9B) in both the training and testing sets. Values are replotted from FIG. 9C, and lines are drawn between the same participants. Significance of difference in distances between the CNN individuality and consistency models was analyzed with paired t-tests.

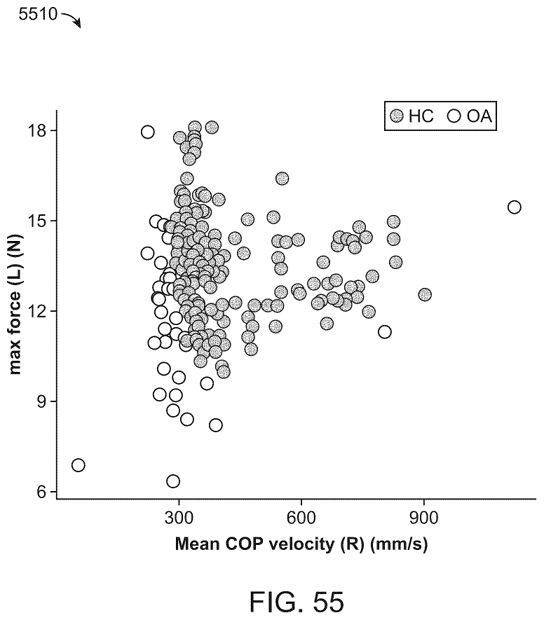

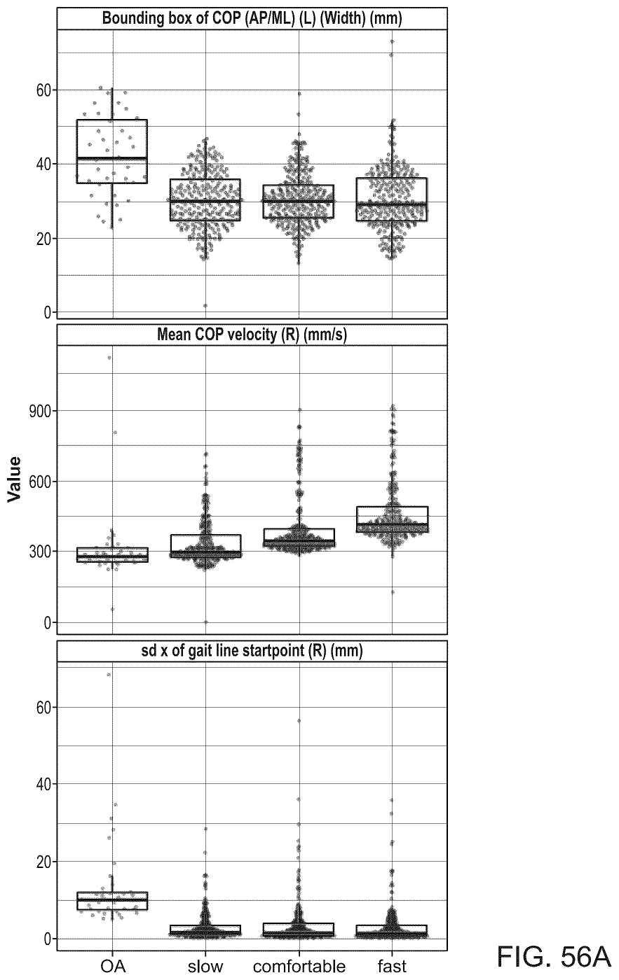

FIG. 55 shows derived gait characteristics 5510 that are most discriminative of knee OA vs controls include Takeoff dynamics, max force (N), Mean COP velocity (mm/s), and sd x of gait line startpoint (mm).

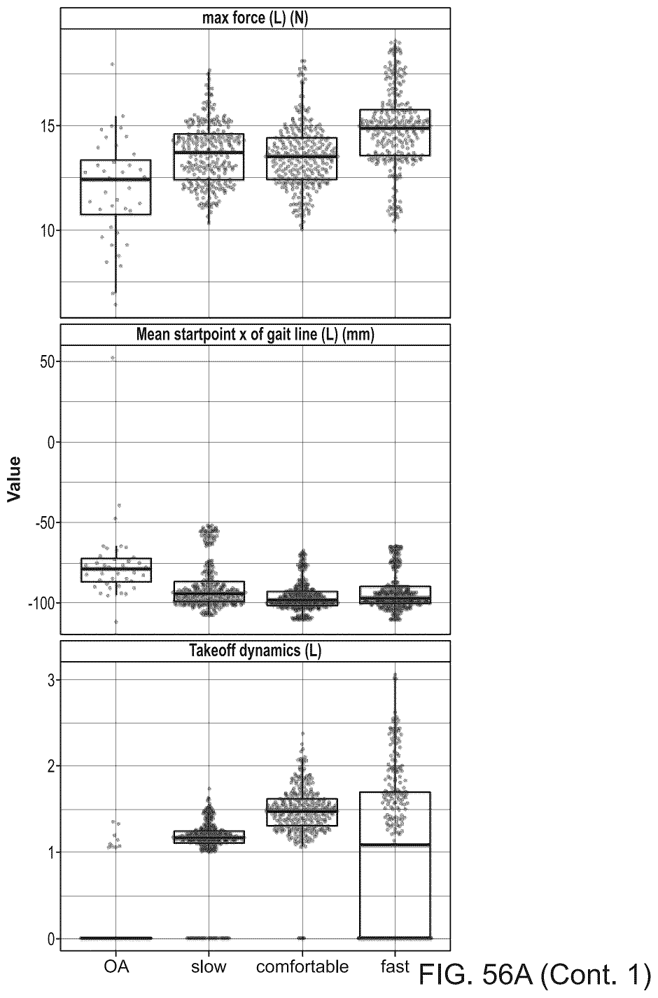

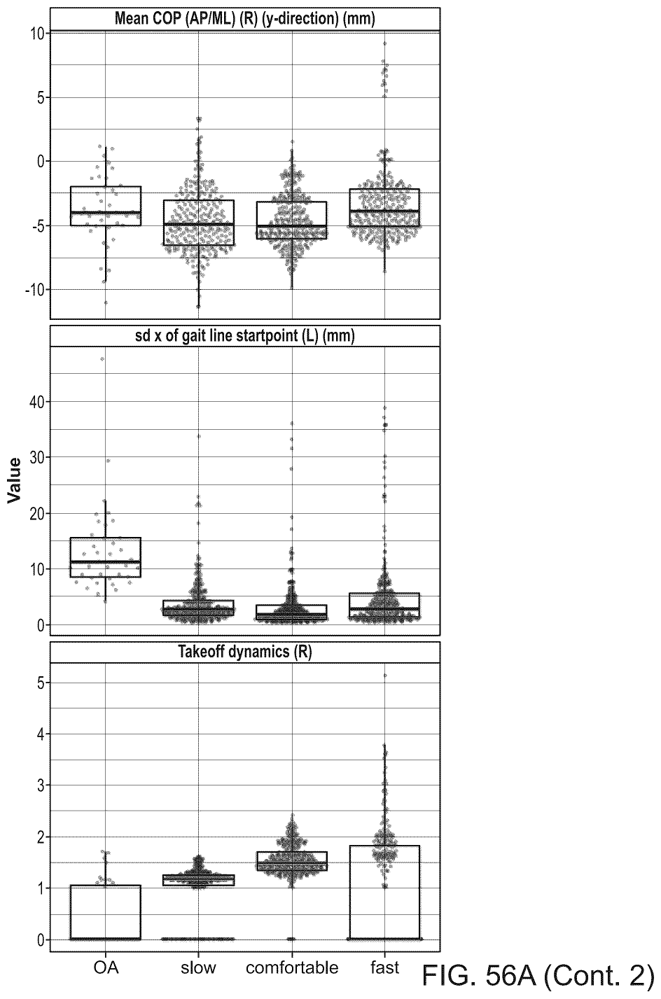

FIGS. 56A-56B show additional examples of derived gait characteristics that are most discriminative of knee osteoarthritis (OA) versus controls include features shown in Supplementary Table 1 (below). FIG. 56A depicts boxplots in knee OA, control slow, comfortable, and fast walking speeds for some parameters indicative of knee OA versus controls. FIG. 56B depicts scatter plots of select parameters in HC (healthy control) versus OA at comfortable walking speed.

Supplementary Table 1 shows derived gait characteristics that may be associated and/or found to be important in a machine learning (ML) model to differentiate control from knee osteoarthritis (OA) subjects. For the control vs OA comparisons shown in Supplementary Table 1 below, a column indicating FDR corrected p values (adjusted for multiple comparisons) is provided. In general, it should be appreciated that corresponding language has been included indicating nominal vs FDR adjusted p values. Nominal p values may still be quite informative in such contexts, even if they do not meet experiment wide significance.

SUPPLEMENTARY TABLE 1

| | | | | OA v | | | |
|---|---|---|---|---|---|---|---|
| | | | | HC | | | |
| | | Average | Average | t-test | XGBoost | | |
| Derived gait | | value in | value in | nominal | feature | | |
| characteristic | Foot | HC | OA | P value | importance | Category | Description |
| Bounding box of | left | 30.18 | 41.84 | 3.69E−08 | 0.03 | COP | |
| COP (AP/ML) (L) | | | | | | | |
| (Width) (mm) | | | | | | | |
| Force raise (L) | left | 0.28 | 0.51 | 1.09E−11 | 0.02 | dynamics | Time (in s) |
| (s) | | | | | | | to first peak |
| Force raise (R) | right | 0.33 | 0.49 | 6.52E−07 | 0.01 | dynamics | of the mean |
| (s) | | | | | | | force curve |
| | | | | | | | after initial |
| | | | | | | | contact. |
| | | | | | | | Mean of all |
| | | | | | | | complete |
| | | | | | | | steps. |
| Forefoot/backfoot | left | 0.47 | 0.49 | 7.99E−04 | 0.00 | coordination | |
| dominance (L) | | | | | | | |
| max force (L) (N) | left | 13.97 | 11.93 | 5.36E−06 | 0.10 | GRF | |
| Mean COP | right | −4.21 | −3.82 | 3.76E−01 | 0.03 | COP | |
| (AP/ML) (R) | | | | | | | |
| (y-direction) | | | | | | | |
| (mm) | | | | | | | |
| Mean COP | right | 396.67 | 312.29 | 2.59E−03 | 0.09 | COP | Mean |
| velocity (R) | | | | | | | velocity of |
| (mm/s) | | | | | | | the center |
| | | | | | | | of pressure |
| | | | | | | | (COP) |
| | | | | | | | during |
| | | | | | | | stance |
| | | | | | | | phase. The |
| | | | | | | | faster the |
| | | | | | | | foot rolls off, |
| | | | | | | | the higher |
| | | | | | | | the gait line |
| | | | | | | | velocity. |
| Mean endpoint y | right | −2.12 | 0.44 | 1.04E−02 | 0.00 | gait line | |
| of gait line (R) | | | | | | | |
| (mm) | | | | | | | |
| Mean max force | right | 13.88 | 13.08 | 2.10E−01 | 9.93E−05 | GRF | Maximum |
| (R) (N) | | | | | | | ground |
| | | | | | | | reaction |
| | | | | | | | force during |
| | | | | | | | stance |
| | | | | | | | phases. |
| | | | | | | | Mean of all |
| | | | | | | | complete |
| | | | | | | | steps |
| Mean start point | left | −93.25 | −76.25 | 8.82E−05 | 0.02 | gait line | |
| x of gait line (L) | | | | | | | |
| (mm) | | | | | | | |
| Mean start point | right | −96.78 | −82.78 | 1.09E−03 | 0.00 | gait line | |
| x of gait line (R) | | | | | | | |
| (mm) | | | | | | | |
| Medial-/lateral | left | 0.47 | 0.46 | 5.65E−01 | 0.01 | coordination | |
| Dominance (L) | | | | | | | |
| Medial-/lateral | right | 0.47 | 0.49 | 3.99E−03 | 0.00 | coordination | |
| Dominance (R) | | | | | | | |
| sd of stance | right | 0.07 | 0.10 | 6.14E−04 | 0.00 | temporal | |
| duration (R) (s) | | | | | | | |
| sd of swing | right | 0.03 | 0.06 | 1.59E−02 | 0.02 | temporal | When |
| duration (R) (s) | | | | | | | standard |
| sd x of gait line | left | 3.98 | 12.95 | 5.10E−09 | 0.09 | gait line | deviations |
| start point (L) | | | | | | | are given, |
| (mm) | | | | | | | then the |
| sd x of gait line | right | 3.26 | 13.04 | 3.09E−06 | 0.06 | gait line | notion is |
| start point (R) | | | | | | | always “SD |
| (mm) | | | | | | | across |
| sd y of gait line | left | 0.87 | 2.07 | 5.01E−09 | 0.02 | gait line | steps”. |
| start point (L) | | | | | | | These |
| (mm) | | | | | | | values |
| | | | | | | | describe the |
| | | | | | | | variability of |
| | | | | | | | gait. |
| sd y of gait line | right | 0.86 | 2.24 | 1.31E−06 | 0.00 | gait line | |
| start point (R) | | | | | | | |
| (mm) | | | | | | | |
| Take-off | left | 1.16 | 0.26 | 1.03E−14 | 0.28 | dynamics | Time to first |
| dynamics (L) | | | | | | | peak of the |
| Take-off | right | 1.14 | 0.36 | 4.61E−10 | 0.20 | dynamics | mean force |
| dynamics (R) | | | | | | | curve after |
| | | | | | | | initial |
| | | | | | | | contact. |
| | | | | | | | Mean of all |
| | | | | | | | complete |
| | | | | | | | steps. |

It should be appreciated that the terms “COP” referred to in the table above may correspond to a center of pressure, “GRF” may correspond to ground reaction force, and “HC” may correspond to healthy control.

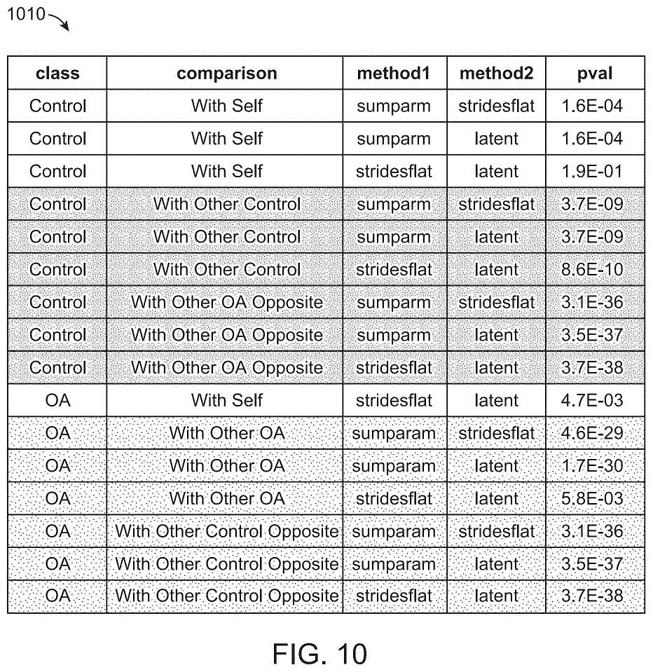

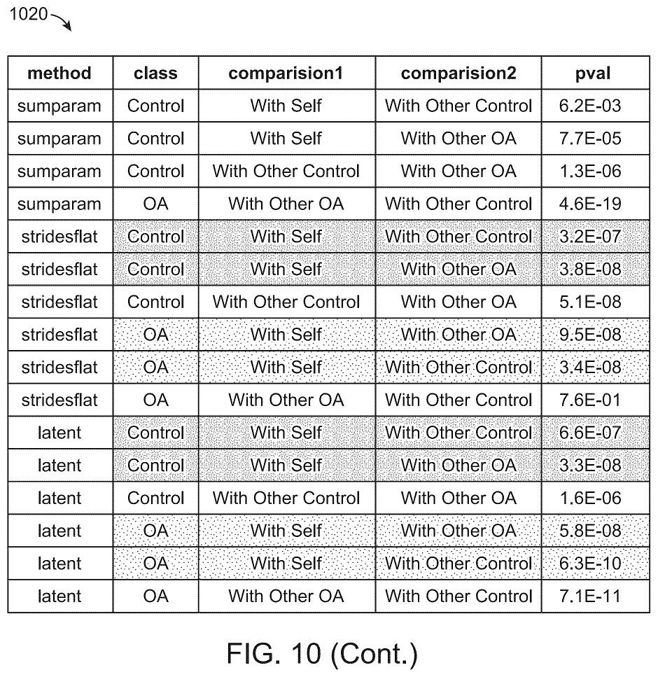

In accordance with the implementations disclosed herein, FIG. 10 shows charts 1010 and 1020. Chart 1010 shows data segregated based on class, comparison metric, a first method, a second method, and corresponding pvals. Chart 1020 shows data segregated based on a method, class, a first comparison, a second comparison, and corresponding pvals.

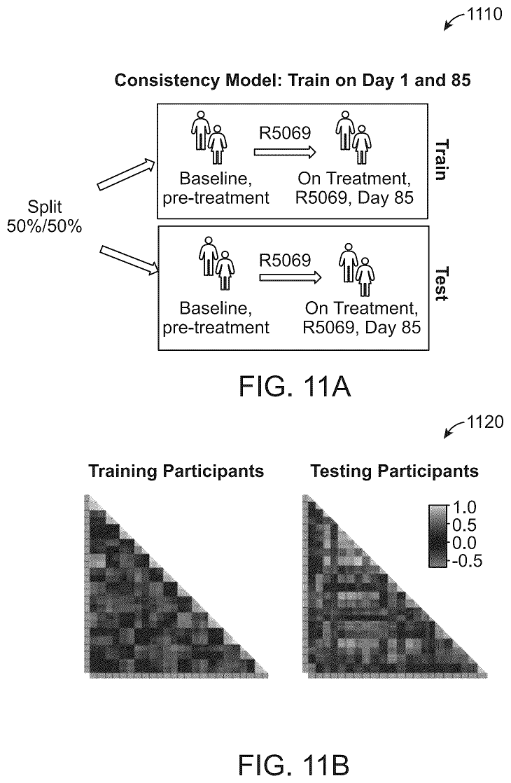

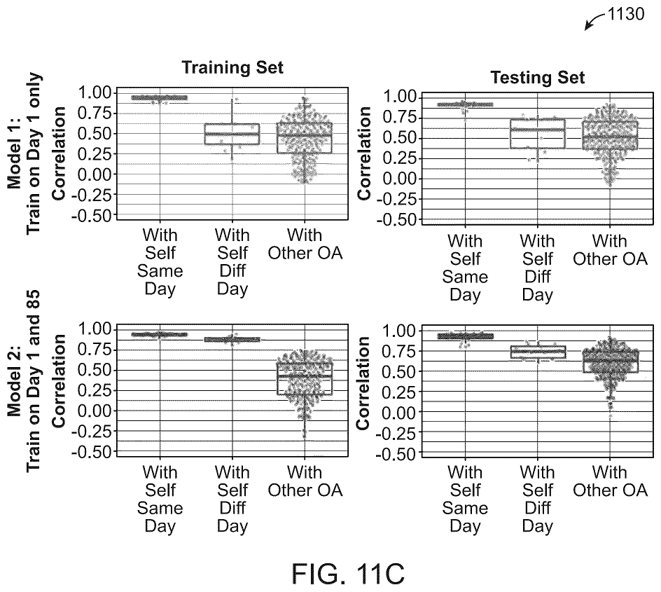

In accordance with the implementations disclosed herein, FIGS. 11A-11C show that training across multiple days increases consistency of CNN model representations of the same participants from different time points, as disclosed herein. FIG. 11A shows a consistency model 1110 on Day 1 and Day 85 split 50:50 between training and test data. FIG. 11B shows spearman data 1120 plotted for training participants and testing participants. FIG. 11C shows box charts 1130 for two models across a training set and a testing set.

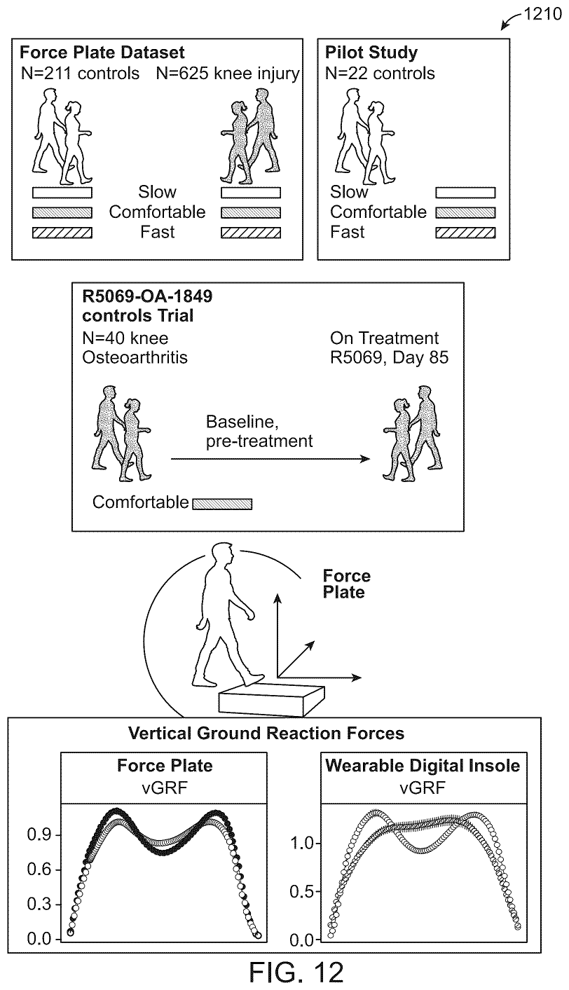

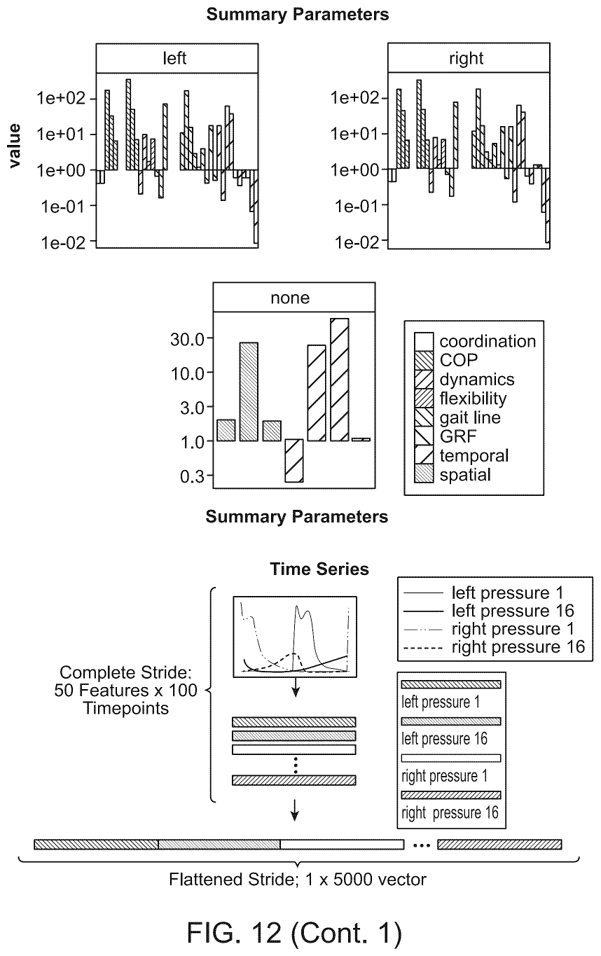

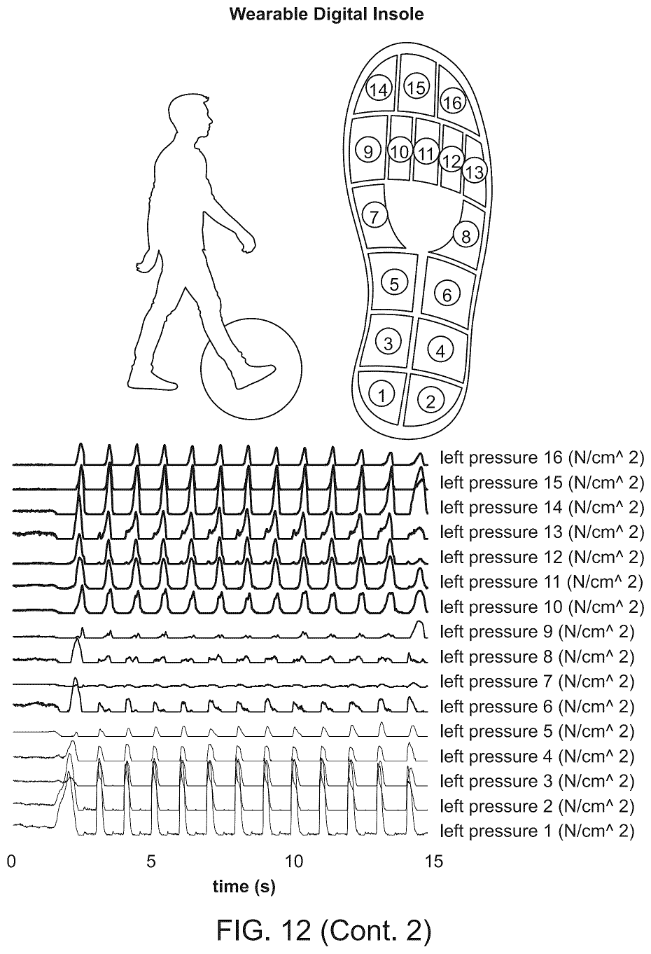

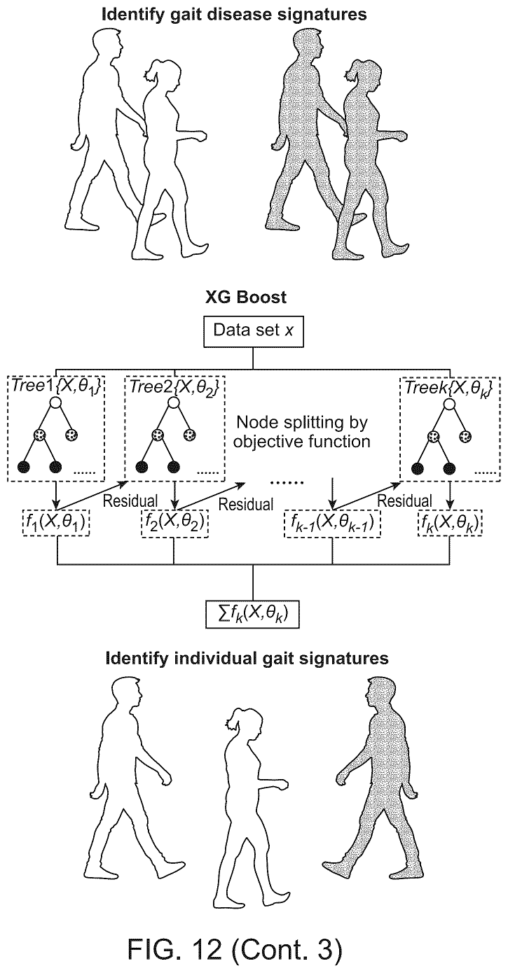

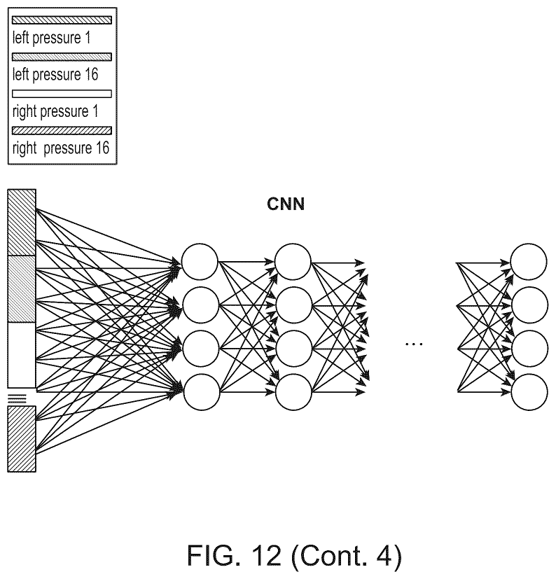

In accordance with the implementations disclosed herein, FIG. 12 shows an overview 1210 of pollutions used in the studies disclosed herein. FIG. 12 includes data similar to FIGS. 4A-4D disclosed herein.

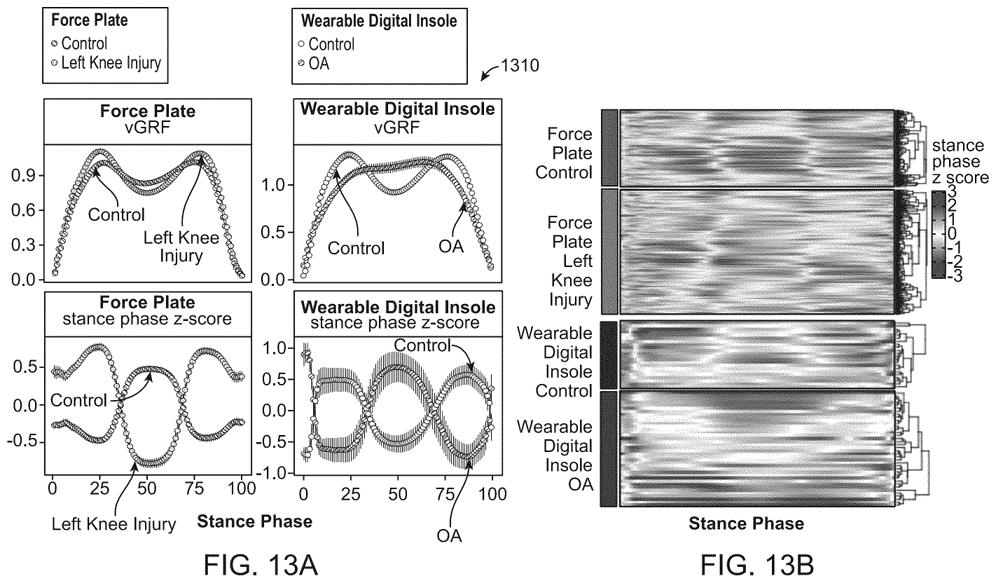

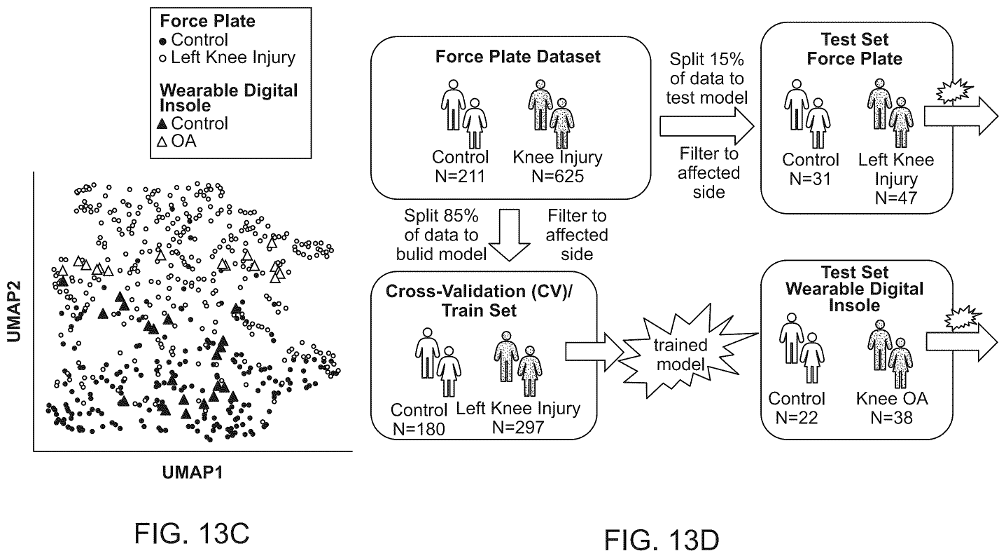

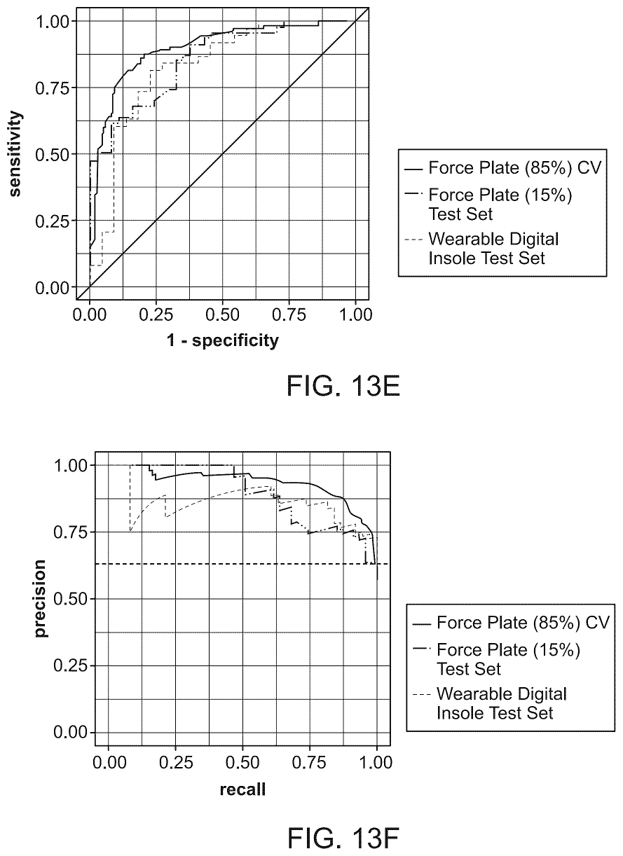

In accordance with the implementations disclosed herein, FIGS. 13A-13F show vGRF data measured by production force plates and test wearable devices to distinguish control from knee injury. FIG. 13A shows vGRF curves derived from force plate (production device) and wearable (digital) insole device (test device) data for healthy controls, and knee injury and knee OA subjects, respectively. Left foot data 1310 are shown as mean of values (top panels) and mean of normalized z-scores (bottom panels) at each percent stance phase within each device and health status. Groups are coded by corresponding visual characteristics (e.g., shading) as in FIGS. 13B and 13C.

FIG. 13B shows vGRF curves for each individual's foot (e.g., left foot) shown as heat map rows, after data was z-transformed to generate Z scores at each percent stance phase (as in FIG. 13A). The rows of FIG. 13B are hierarchically clustered within each group of subjects. FIG. 13C shows a UMAP dimensionality reduction of the z-transformed foot (e.g., left foot) vGRF data. Each point in FIG. 13C represents a subject, and points are colored by phenotype, and shaped by device.

FIG. 13D shows a schematic of machine learning model building of training/validation and testing sets. Two XGBoosts models may be created, one for left knee injury (depicted in FIG. 13D) and one for right knee injury. The full force plate vGRF dataset with both controls (e.g., comfortable walking speed) and left or right knee injury subjects (e.g., comfortable walking speed, excluding subjects with knee injury on both joints) may be split 85% into training/validation datasets, and 15% into hold-out testing set. One model may determine control versus knee injury subjects using left foot data (of left knee injury subjects and all controls), and another may determine using right foot data (of right knee injury subjects and all controls). These models may be applied on a separate and/or independent testing set of digital insoles vGRF data with N=22 control subjects and N=38 patients with knee OA. FIG. 13E shows a receiver operating characteristic (ROC) curve for XGBoost classification, such as of force plate (85%) cross-validation (CV, training/validation) set, force plate (15%) hold-out test set, and digital insole test set. FIG. 13F shows a precision-recall curve for XGBoost classification of the same groups shown in FIG. 13E.

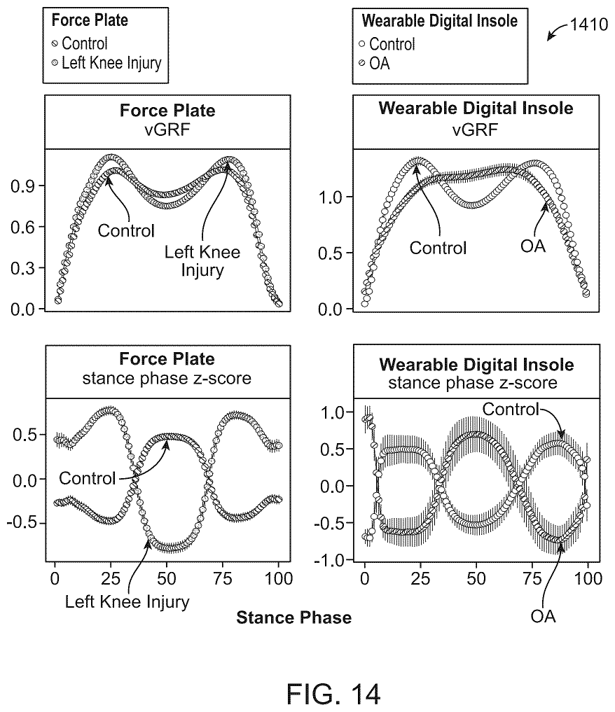

In accordance with the implementations disclosed herein, FIG. 14 shows vGRF curves 1410 from control and knee injury populations to indicate that such data can be measured using both force plate and wearable insole devices. FIG. 14 may be used to understand how vGRF data collected from a wearable device compared to gold standard vGRF data collected using force plates (clinical gold standard). The datasets include GaitRec Force Plate dataset of N=211 controls at comfortable walking speed, N=625 knee injury subjects with 344 subjects with left knee injuries, wearable device control: Pilot study of N=22 controls for 30 second walking intervals, and wearable device OA: R5069-OA-1849 clinical trial of N=40 OA subjects at 3-minute walking intervals. Averaged vGRF curves are similar across platforms (top panels), column normalization within device bring different platforms onto same scale (bottom panels), and qualitative observation show that individuals with affected knees look different than healthy controls (“flatter” vGRF curve shape during the middle of stance phase)

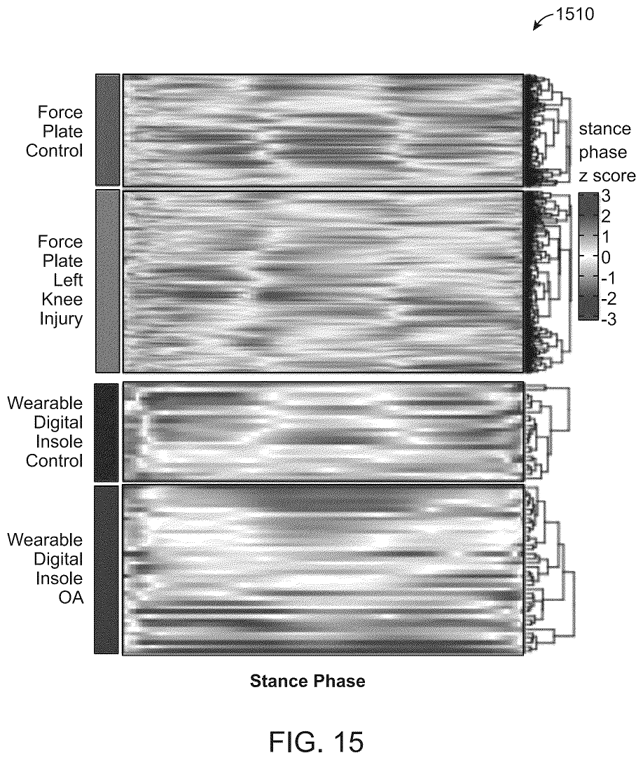

In accordance with the implementations disclosed herein, FIG. 15 shows z-scored vGRF curves 1510 on a per subject basis that indicate differences between control and knee injury using both force plates and wearable devices. FIG. 15 may be used to understand how vGRF data collected from a wearable device compared to gold standard vGRF data collected using force plates (clinical gold standard). The datasets include GaitRec Force Plate datasets N=211 controls at comfortable walking speed, N=344 left knee injury subjects, wearable device control pilot study of N=22 controls for 30 second walking intervals, and wearable device OA: R5069-OA-1849 clinical trial of N=40 OA subjects at 3-minute walking intervals. Individual vGRF curves are similar across platforms. Distinct patterns between healthy individuals and those with injured knees.

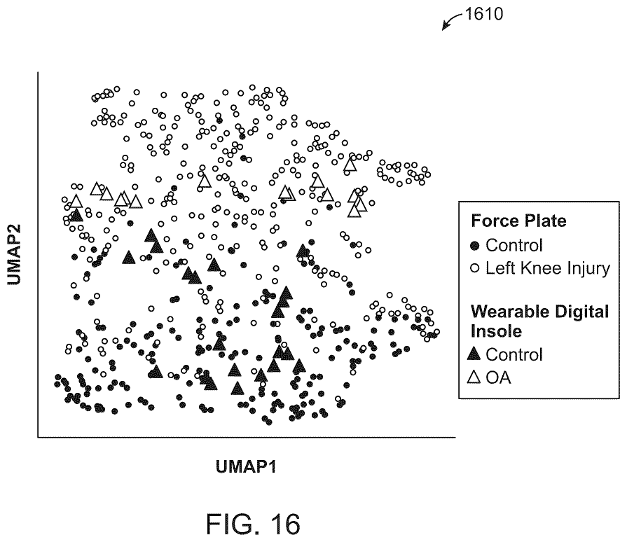

In accordance with the implementations disclosed herein, FIG. 16 shows a UMAP generated using vGRF data from each subject, and shows platform independent clustering between knee injury subjects measured with force plates and knee OA subjects measured with a wearable device. FIG. 16 may be used to understand how vGRF data collected from a wearable device compared to gold standard vGRF data collected using force plates (clinical gold standard). The datasets include GaitRec Force Plate datasets N=211 controls at comfortable walking speed, N=344 left knee injury subjects, wearable device control pilot study of N=22 controls for 30 second walking intervals, and wearable device OA: R5069-OA-1849 clinical trial of N=40 OA subjects at 3-minute walking intervals. A UMAP may be used to observe that subjects separate out by knee injuries rather than by platform.

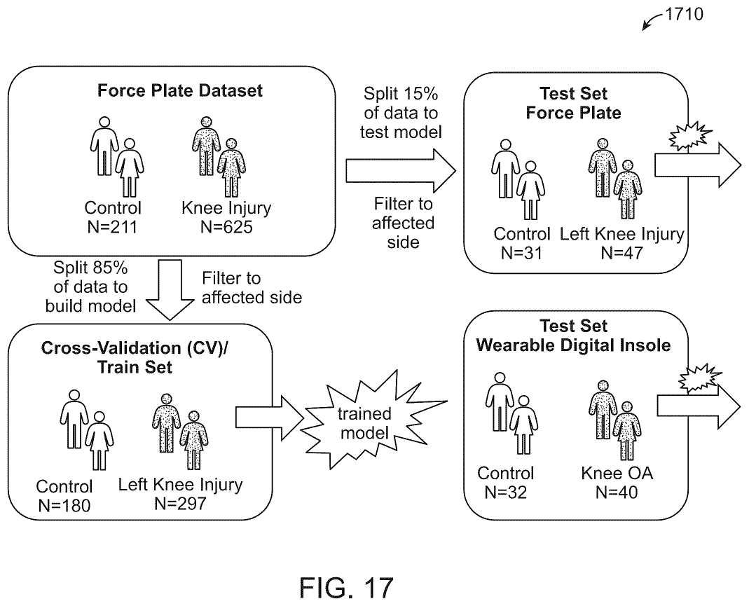

In accordance with the implementations disclosed herein, FIG. 17 shows machine learning model 1710 to understand how well data collected from a wearable device can be used to predict control verses knee injury subjects. Machine learning model 1710 is we built on gold standard force plate data and tested them with unseen force plate and wearable device data. GaitRec force plate vGRF dataset was divided into train/validation set (85%) for the XGBoost model trained test set (15%). The two wearable device datasets also serve as independent datasets.

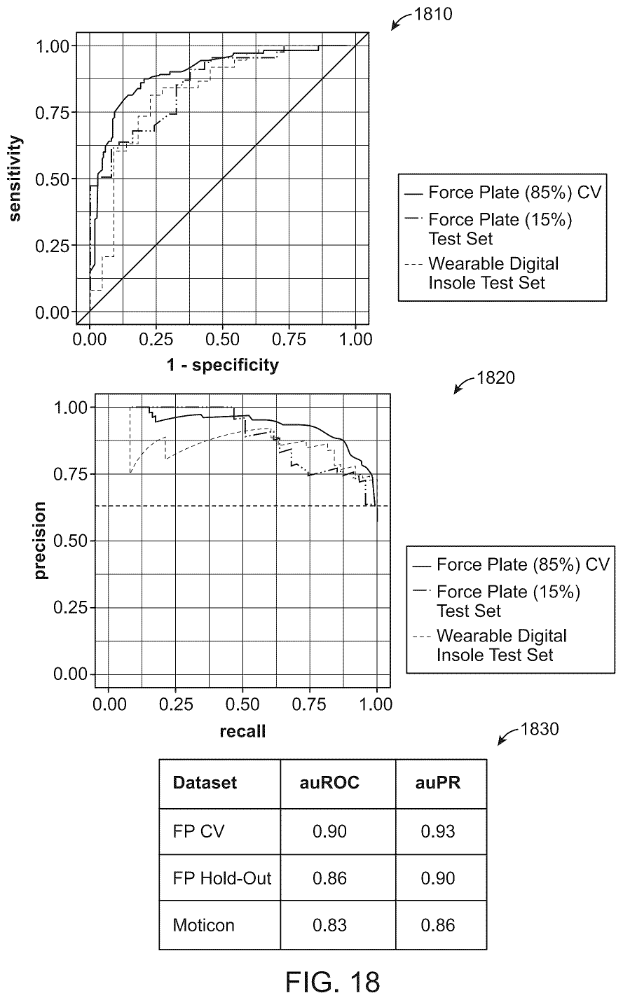

In accordance with the implementations disclosed herein, FIG. 18 shows predictive performance diagrams 1810 and 1820 across force plates and a wearable device, as indicated in chart 1830. Evaluation of trained XGBoost model is based on force plate coefficients of variance: force plate vGRF training set (85%) 5-fold cross-validation, force plate hold-out: hold out force plate vGRF test set (15%), and wearable device vGRF. Model performance is evaluated using auROC=area under receiver operating characteristic (ROC) curve and auPR=area under precision-recall curve.

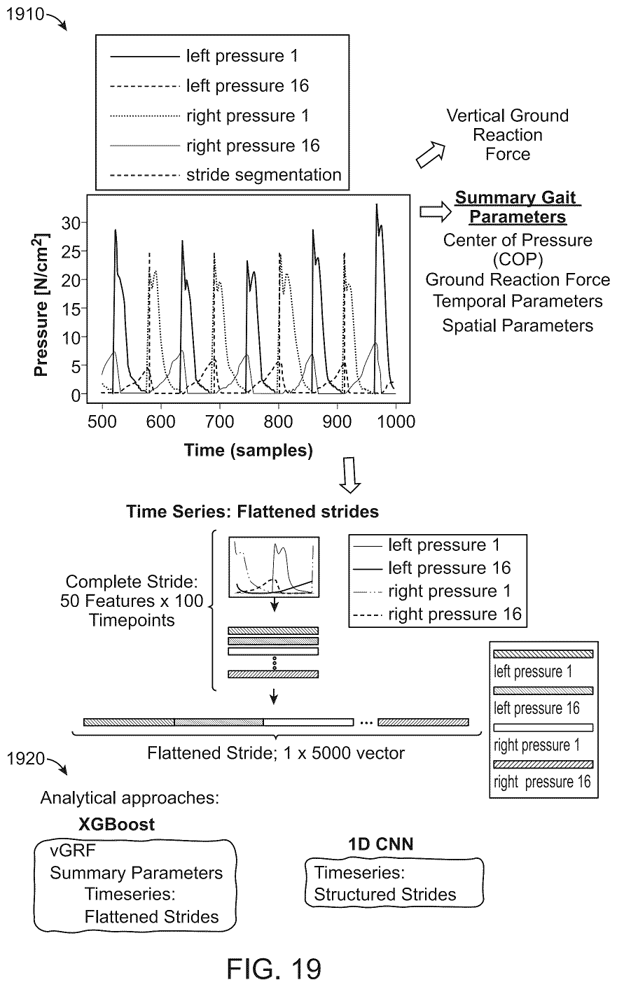

In accordance with the implementations disclosed herein, FIG. 19 shows diagram 1910 and diagram 1920 for an overview of types of data generated using a wearable device. Diagram 1910 shows calculation of vGRF force, gait parameters, and time series data based on sensed data. Diagram 1920 shows analytical approaches including XGBoost and 1D CNN as disclosed herein.

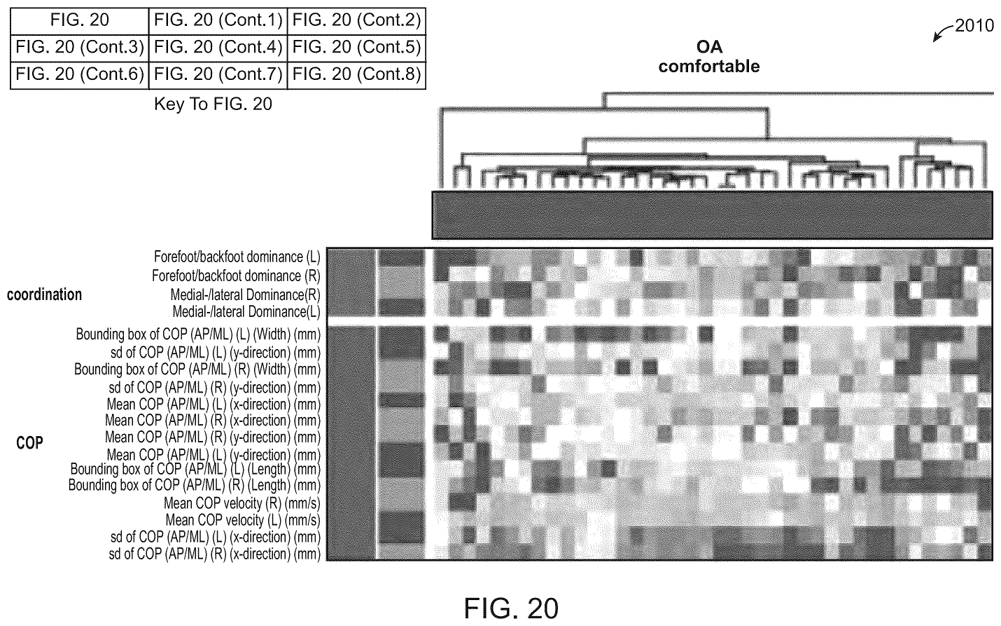

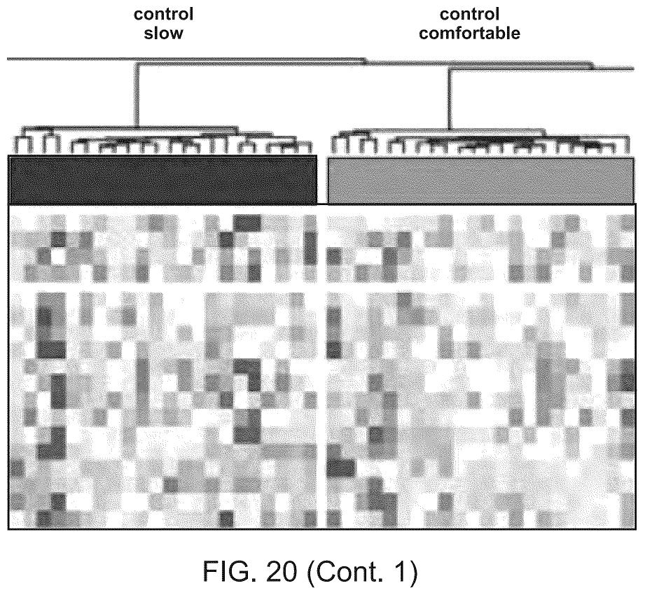

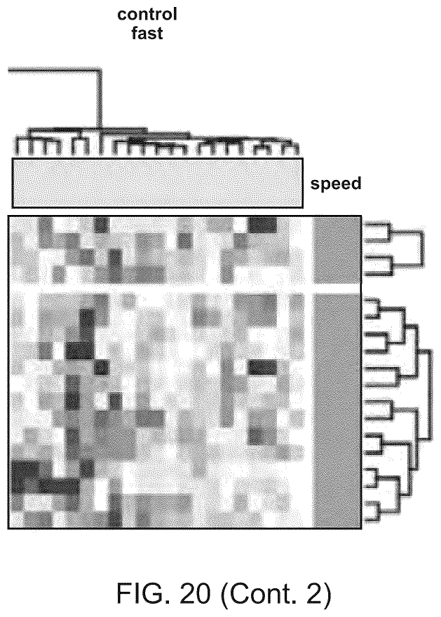

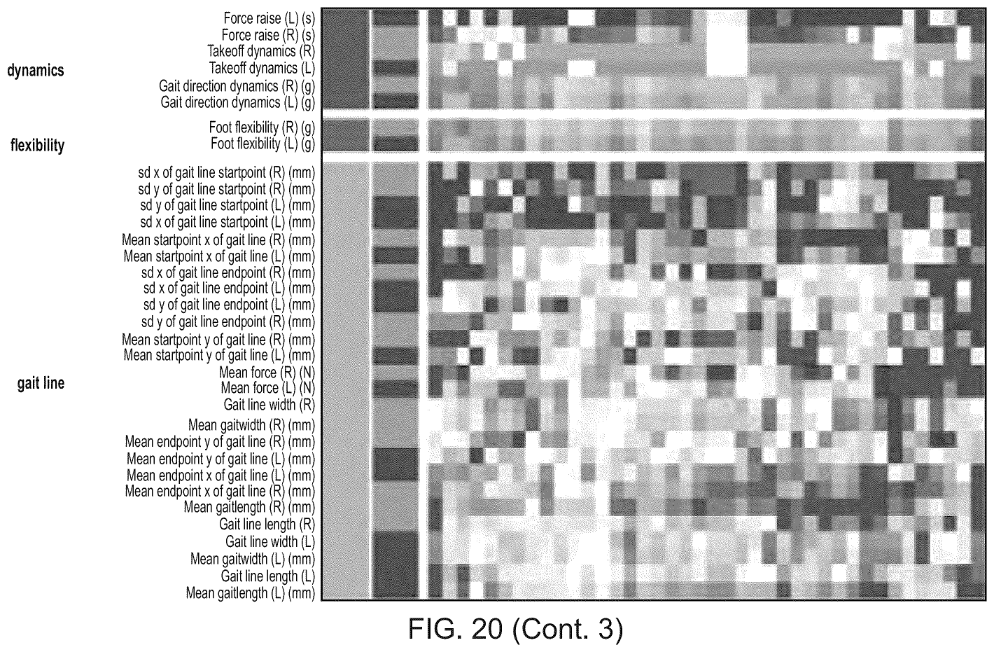

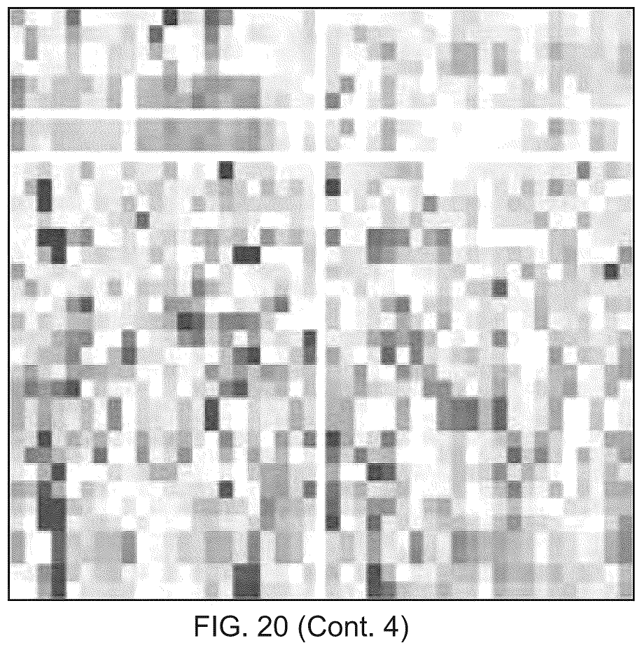

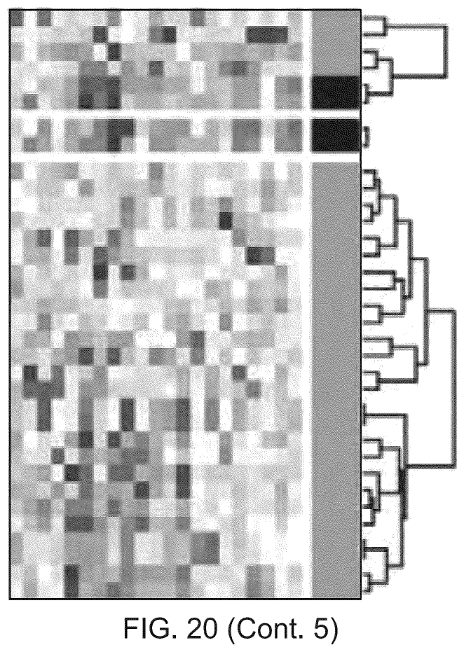

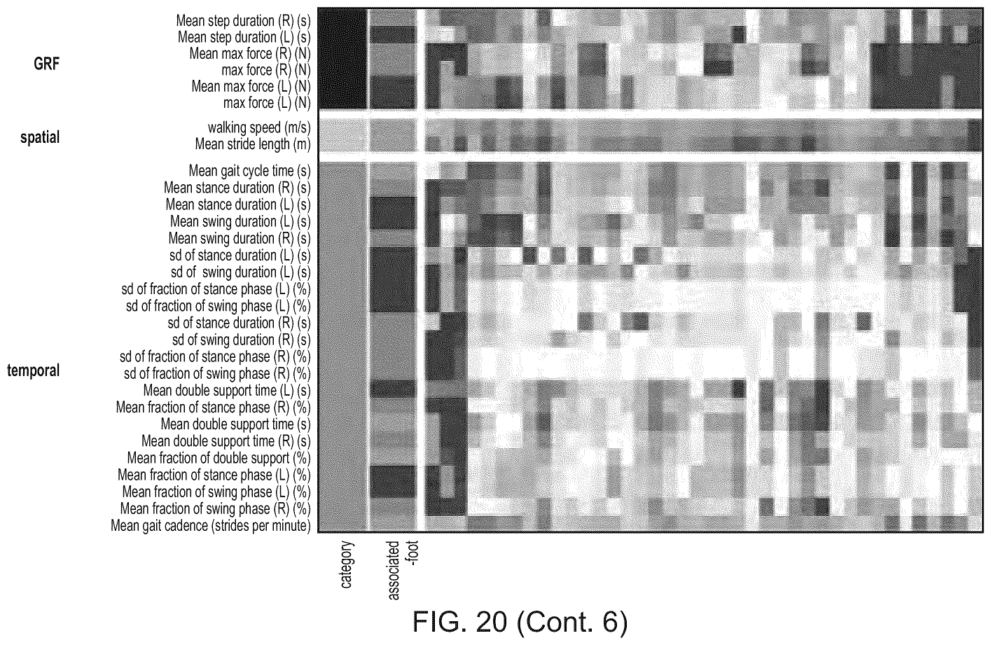

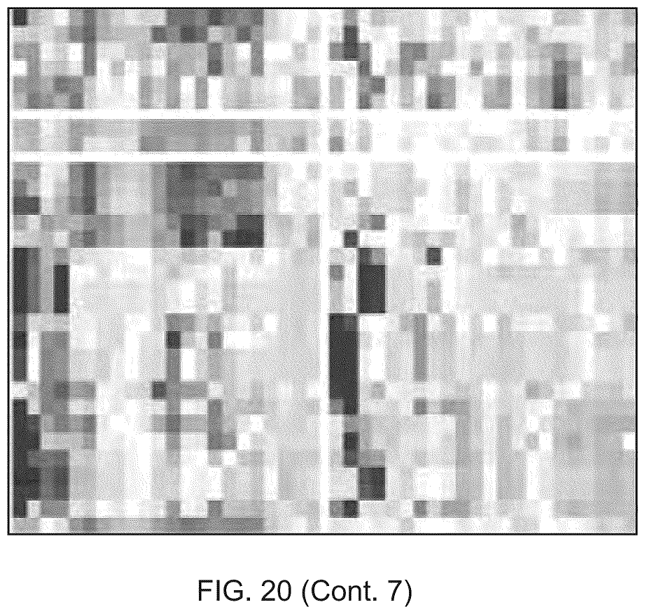

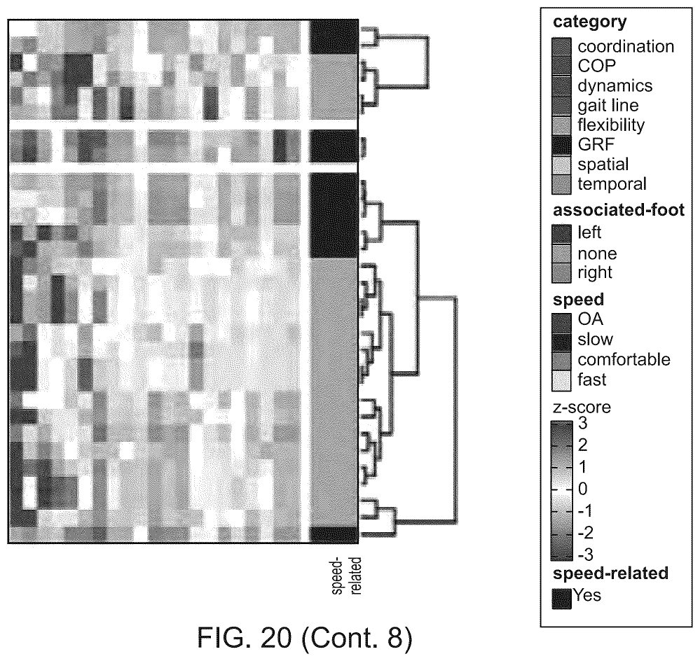

In accordance with the implementations disclosed herein, FIG. 20 shows a heat map 2010 showing distinct patterns that can be observed between summary parameter readouts at different speeds and patient disease status. Summary gait parameters include Summary parameters from the wearable device can be broken down into the following categories: coordination, dynamics, flexibility, gait line, ground reaction forces (grf), spatial, and temporal. Natural clustering is observed with OA/slow/comfortable/fast walks. Different features are used for binary classification of walking speed.

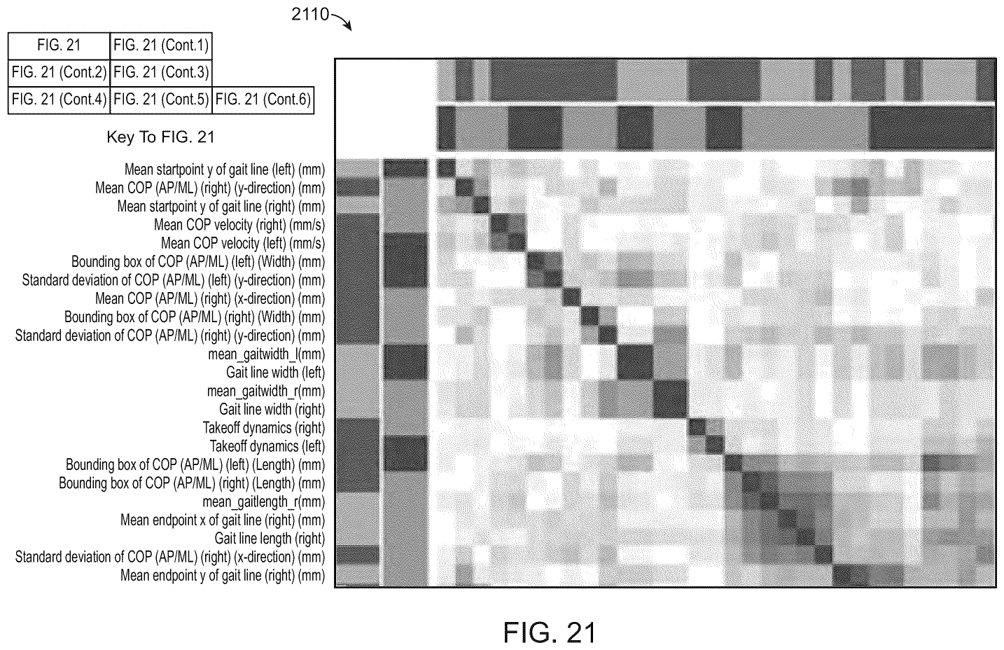

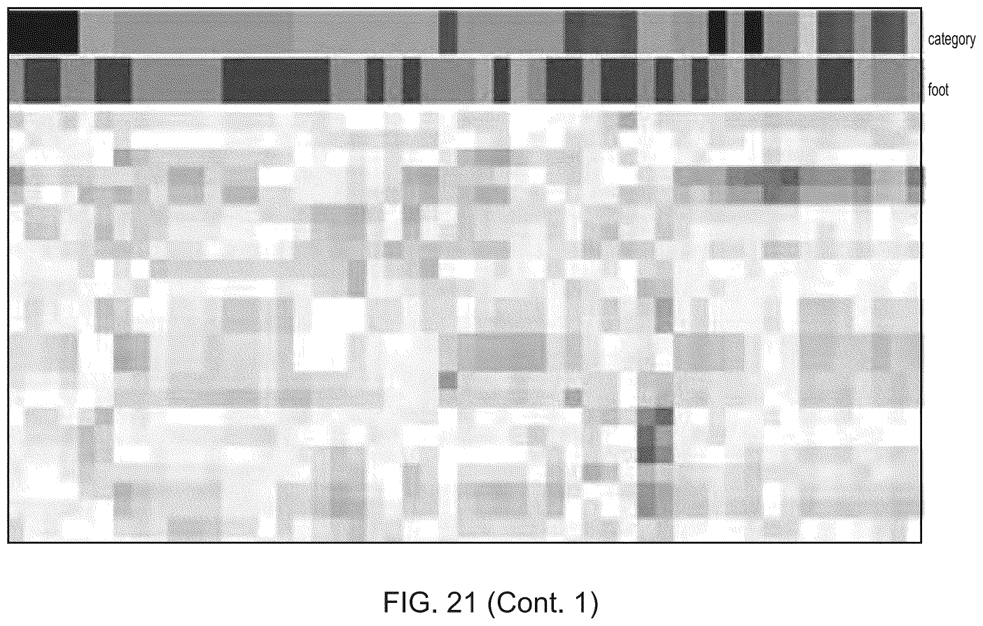

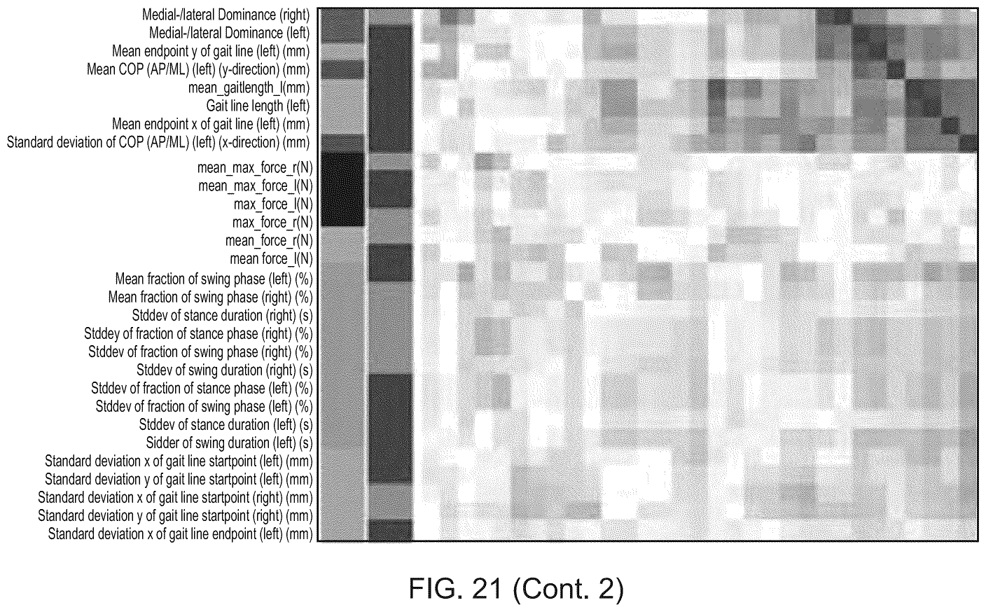

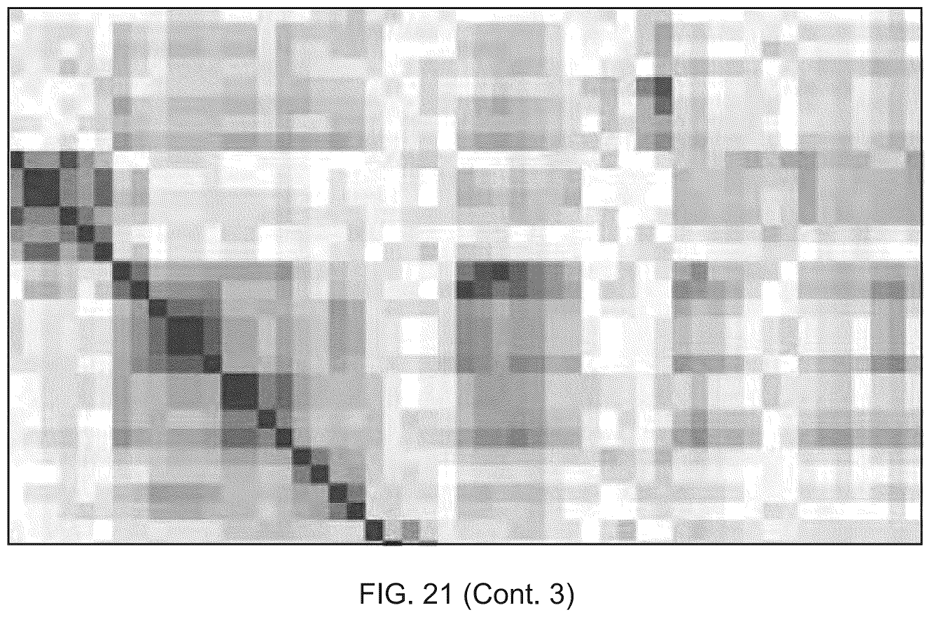

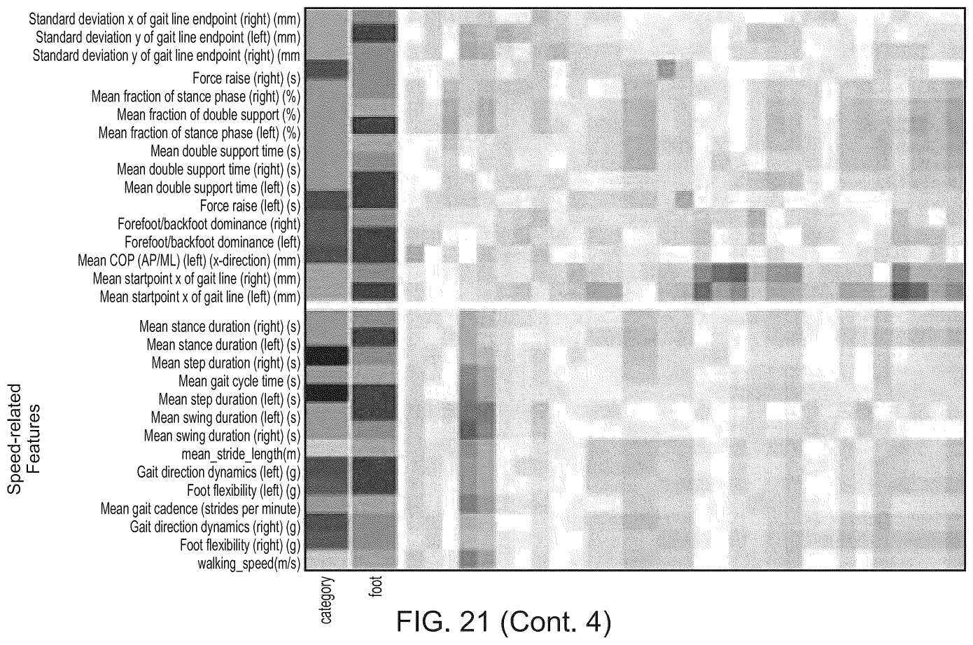

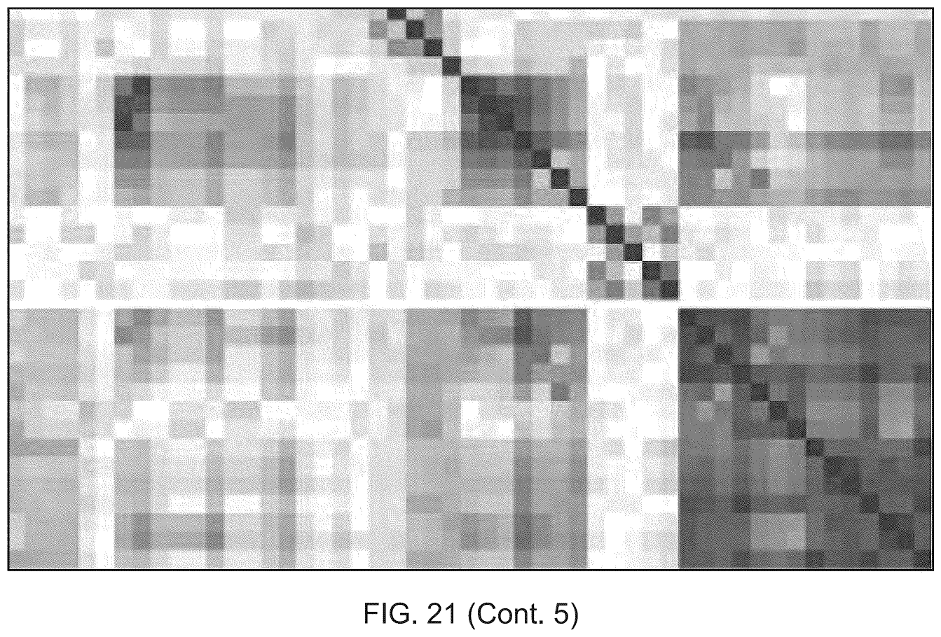

In accordance with the implementations disclosed herein, FIG. 21 shows a heat map 2110 showing correlations within and between categories of summary parameter. Summary gait parameters include 85 summary parameters from the wearable device that can be broken down into the following categories: coordination, dynamics, flexibility, gait line, GRF, spatial, and temporal. A set of features strongly positively and negatively correlated with speed are identified. Positively correlated (rho>0.7): Foot flexibility, Gait direction dynamics, Mean gait cadence (strides per minute), mean stride length. Negatively correlated (rho<−0.7): mean gait cycle time, mean stance duration, mean step duration, mean swing duration.

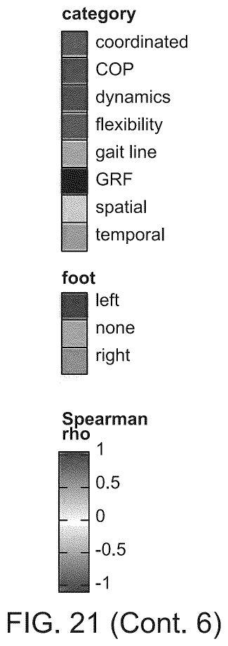

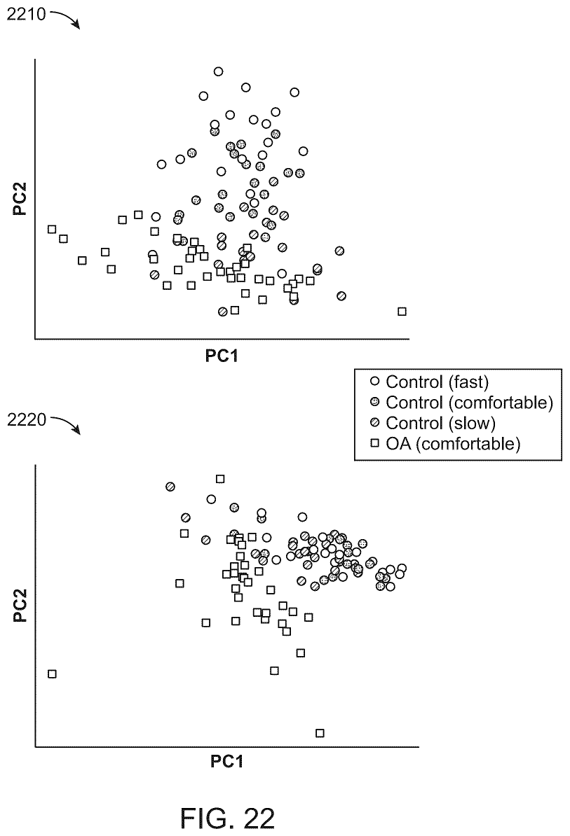

In accordance with the implementations disclosed herein, FIG. 22 shows chart 2210 of vGRF data, chart 2220 of summary gait parameters, and chart 2230 of time series data. Time series data can also be visualized on a per-stride basis for values from the 50 sensors on each foot, as shown in chart 2230 and diagram 1910 of FIG. 19.

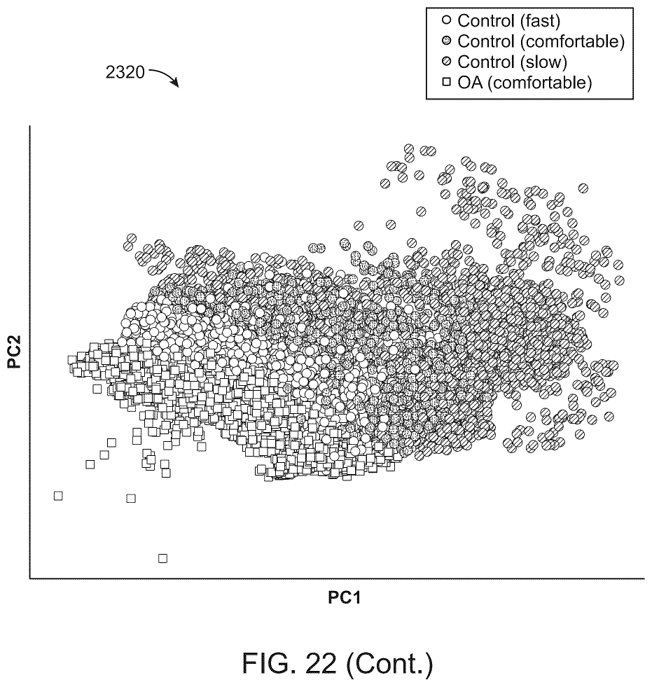

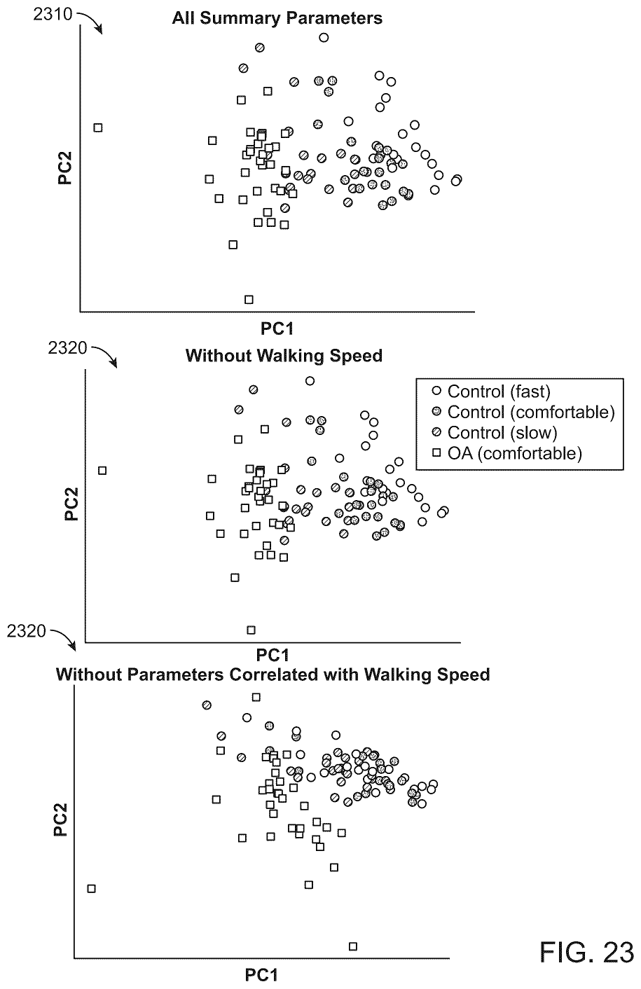

In accordance with the implementations disclosed herein, FIG. 23 depicts charts showing that removal of features correlated with walking speed reduces separation of data points. Chart 2310 shows data points plotted based on all available summary parameters. Chart 2320 shows data points without walking speed included as a parameter. Chart 2330 shows data points without parameters correlated with walking speed.

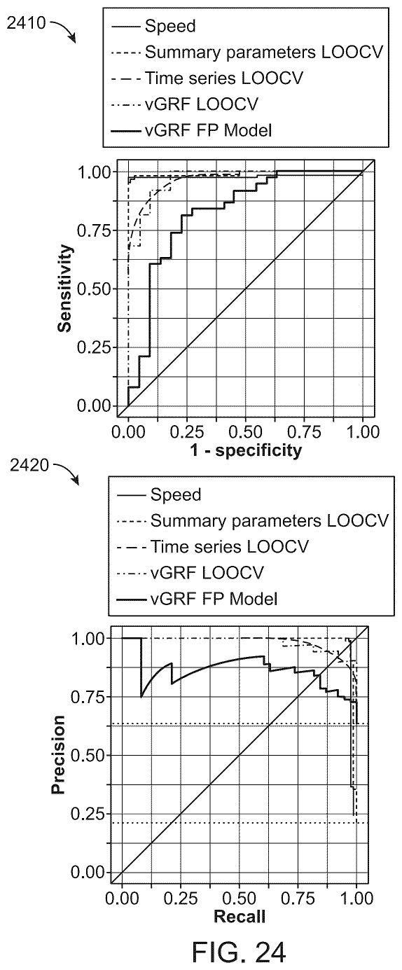

In accordance with the implementations disclosed herein, FIG. 24 depicts charts showing that summary parameters outperform vGRF and time series data when evaluated on a wearable device dataset for knee injury verses control prediction. Chart 2410 shows a wearable device verses comfortable speed data plotted based on specificity and sensitivity. Chart 2420 shows a wearable device verses comfortable speed data plotted based on precision and recall. To facilitate direct comparison of results, models are trained and evaluated using leave-one-out cross validation on the following feature sets: speed alone, summary parameters, time series, and vGRF.

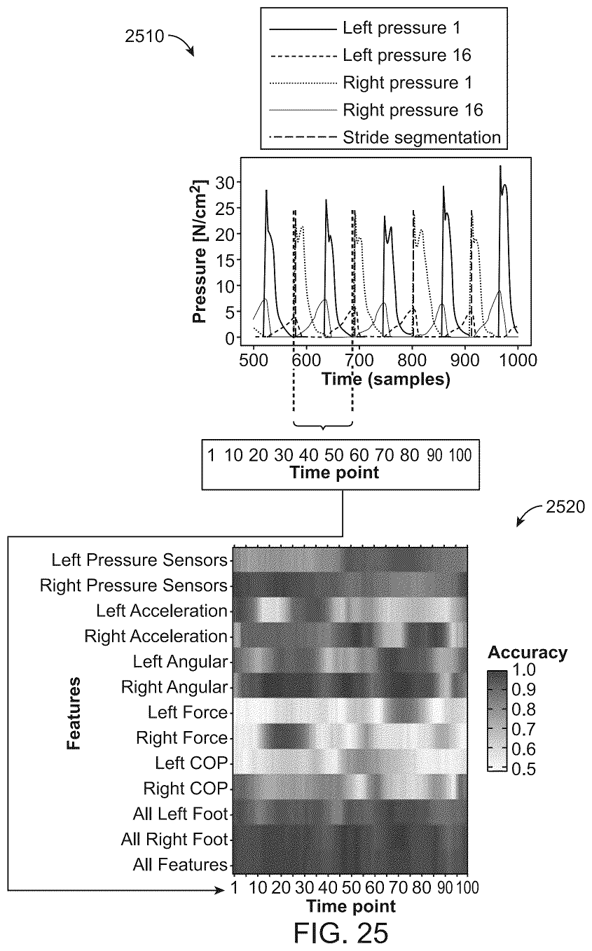

In accordance with the implementations disclosed herein, FIG. 25 shows that classification accuracy differ based on the time point of a stride, as well as the type of sensors. However, user of all features leads to the strongest predictive performance. Chart 2510 show the time point of a stride and heat map 2520 shows a heat map of the wearable device OA verses control comfortable speed accuracy based on features.

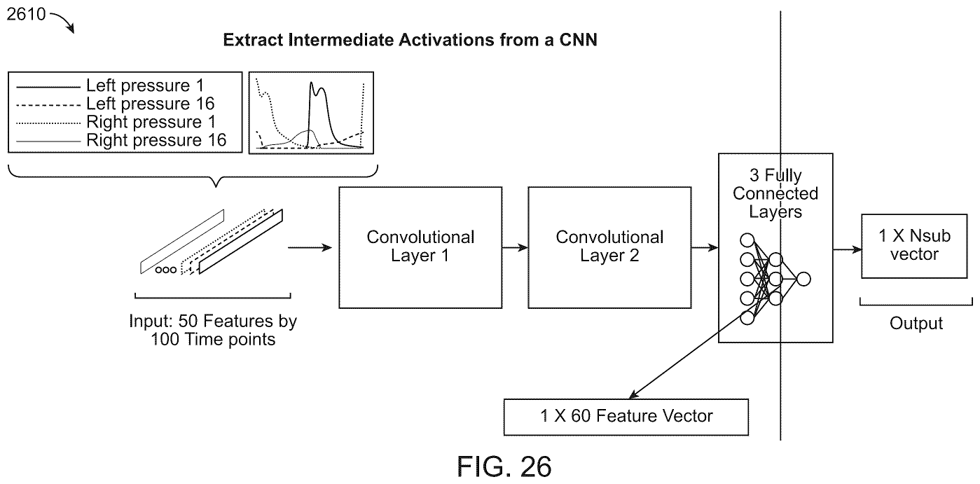

In accordance with the implementations disclosed herein, FIG. 26 shows an overview of how to build a 1D CNN model using gait data. Flow diagram 2610 shows how intermediate activations are extracted from a CNN model. The CNN model, based on gait data, can be used to determine the optimal way to identify a unique gait pattern from an individual that is generalizable to all individuals. This technique can be implemented by building a 1D CNN using a 50×100 time series vector from wearable device sensor data to predict individual subjects (independent of injury class) on a training set. Sixty latent features are extracted from the penultimate layer of the CNN, and individual subject level separation on a held-out test set is identified and/or quantified.

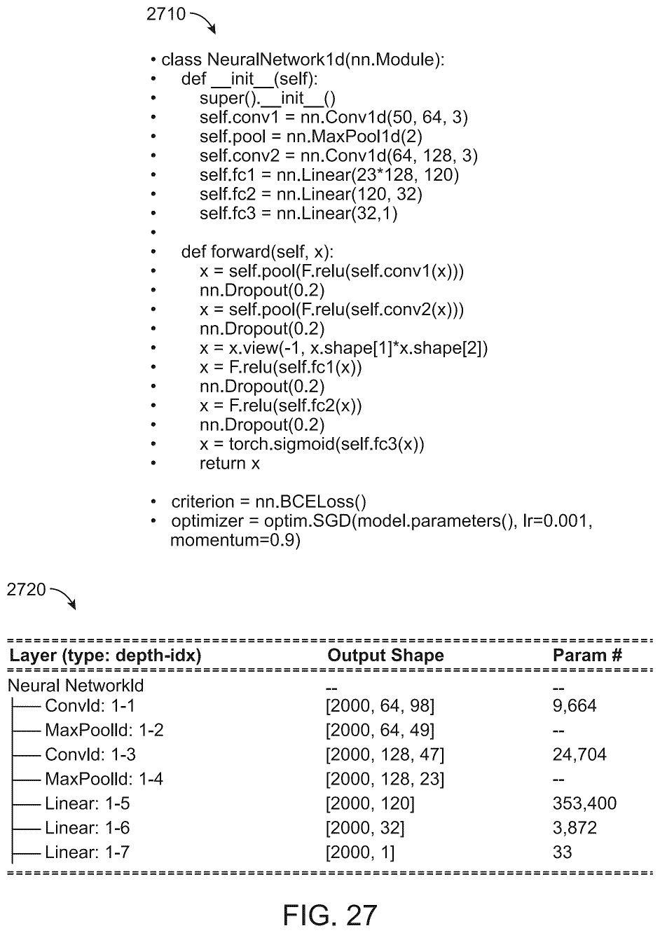

In accordance with the implementations disclosed herein, FIG. 27 shows a CNN architecture having two 1D convolution layers, flattened layer(s), and three fully connected layers. Code 2710 shows how such an architecture can be implemented and table 2720 shows the respective layers, output shape, and person information.

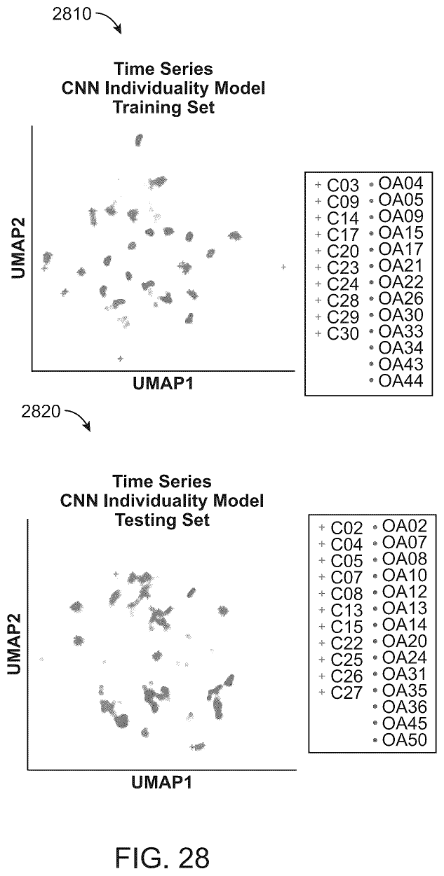

In accordance with the implementations disclosed herein, FIG. 28 shows CNN latent features generated from a training set that predicts subjects on a held-out test set of unseen subjects to show individuality. UMAP 2810 shows a time series CNN individuality model based on a training set and UMAP 2820 shows a time series CNN individuality model based on a testing set. This technique can be implemented using gait data to determine the optimal way to identify a unique gait pattern from an individual, ‘that is generalizable to all individuals by determining individuality of gait patterns. This technique can be implemented by building a 1D CNN using a 50×100 time series vector from wearable device sensor data to predict individual subjects (independent of injury class) on a training set, extracting 60 latent features from the penultimate layer of the CNN, and identifying and/or quantitating individual subject level separation on a held-out test set.

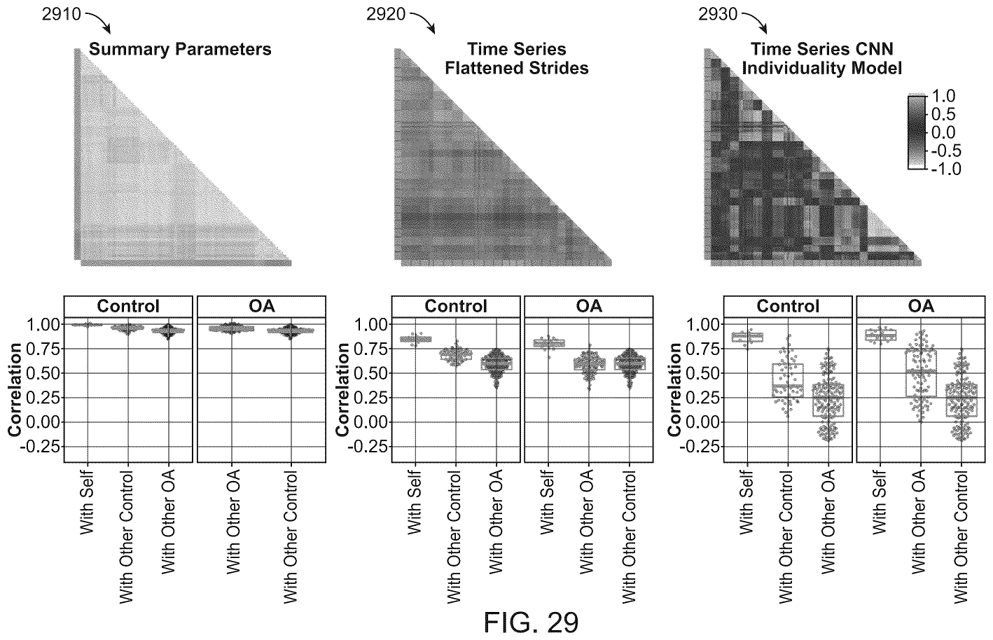

In accordance with the implementations disclosed herein, FIG. 29 shows that latent CNN representations outperform other datatypes at identifying subject-level classification patterns. Summary patterns 2910 are shown using a spearman correlation and box chart. Time series flatten strides 2920 are shown using a spearman correlation and box chart. Time series CNN individuality model 2930 is shown using a spearman correlation and box chart. The OA subjects whose data is shown in FIG. 29 had one 3MWT. Accordingly, no self-assessment for summary parameters is provided.

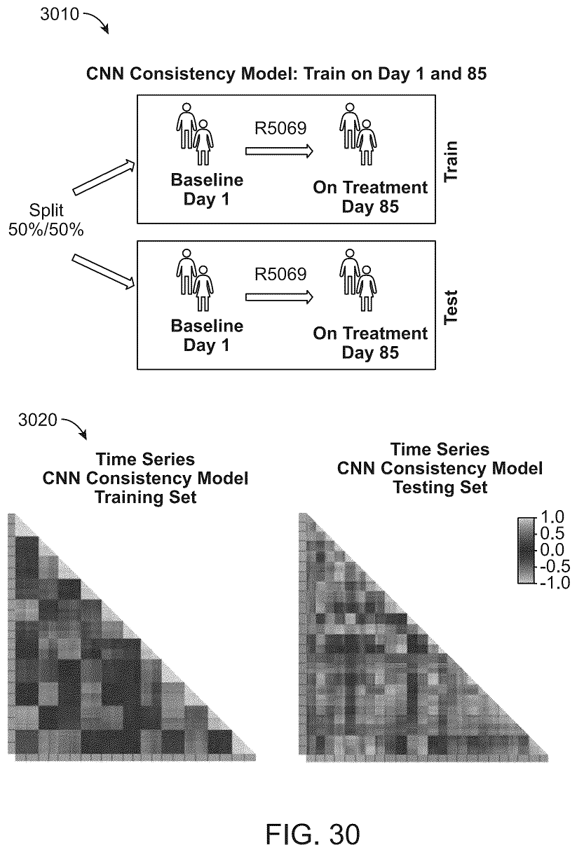

In accordance with the implementations disclosed herein, FIG. 30 shows that training from two days (e.g., day 1 and day 85) may help recognition of consistency. OA subjects with day 1 and day 85 data are split into training and testing sets. The CNN model shown is trained on both days for training participants and is applied to testing participants. Day 1 and day 85 for each participant are grouped and displayed with the same shade, separated by black partition. Diagram 3010 shows a CNN consistency model for training on day 1 and day 85. Diagram 3020 shows a time series CNN consistency model correlation based on a training set and time series CNN consistency model correlation based on a testing set.

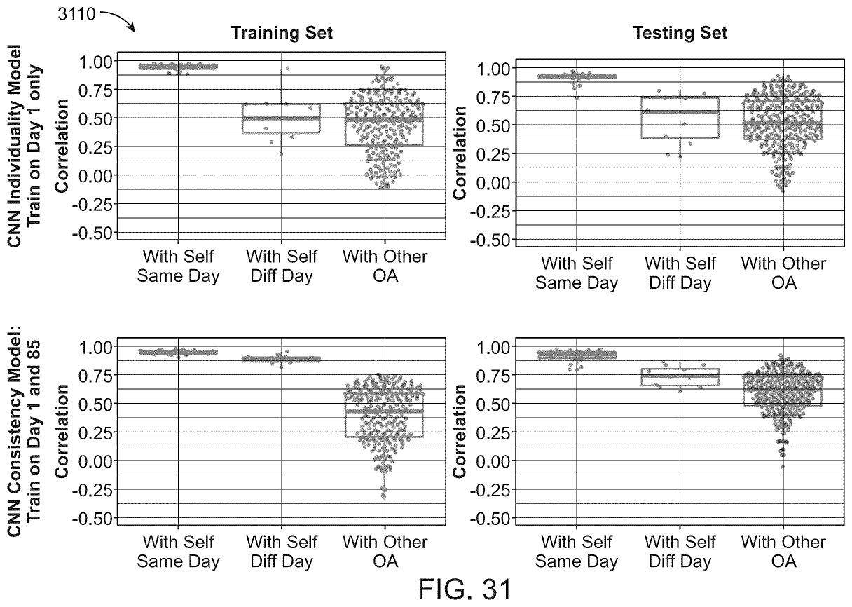

In accordance with the implementations disclosed herein, FIG. 31 shows that training on data from two days (e.g., day 1 and day 85) may help with recognition of consistency. Box charts 3110 show differences between a training set and testing set based on a CNN individuality model trained on day 1 only and a CNN consistency model trained on both day 1 and day 85.

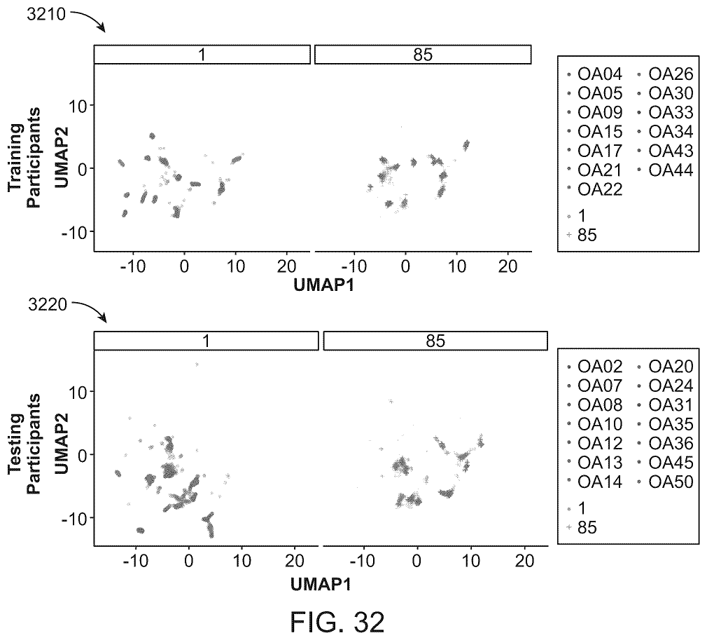

In accordance with the implementations disclosed herein, FIG. 32 shows that latent values from a CNN model trained on a first day's (e.g., day 1) data of training participants does not capture features that are preserved across multiple visits by the same OA individuals. UMAP 3210 corresponds to day 1 and day 85 data for training participants and UMAP 3220 corresponds to day 1 and day 85 testing participants. FIG. 32 may be used to determine the consistency of gait signatures using a 1D CNN. A model may be built using day 1 data and may be tested using day 85 data. As can be shown in UMAP 3210 and 3220, latent features across the days do not correlate as well as latent features within the same day. According to an implementation, a model may be trained on day 1 and day 85 data for a subset (e.g., half) the subjects and may be tested on the remaining subset of subjects.

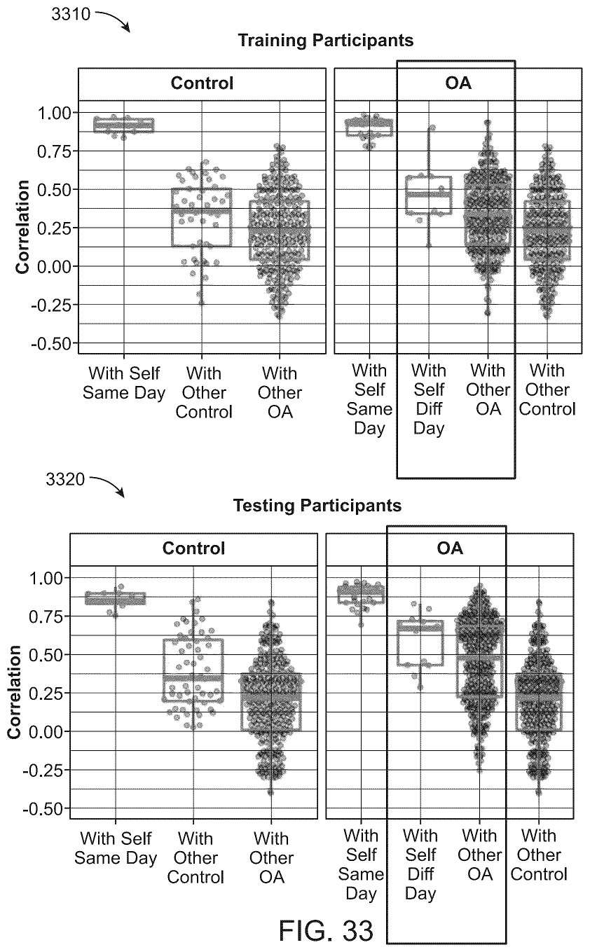

In accordance with the implementations disclosed herein, FIG. 33 shows that CNN latent values from the same OA individuals are significantly different across day 1 and day 85. Box plot 3310 shows correlation data for training participants and box plot 3320 shows correlation data for testing participants. Comparisons of day 1 vs day 1, day 1 vs day 85, day 85 vs day 1, and day 85 vs day 85 of same pairs of individuals are shown as separate dots in “With Other OA” and “With Other Control.” Day 1 of training participants are used to build the CNN model and latent values are obtained for all days for all participants.

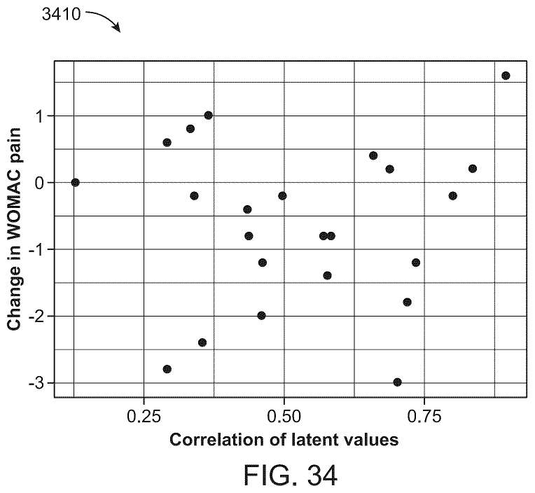

In accordance with the implementations disclosed herein, FIG. 34 includes chart 3410 that shows that correlation of CNN latent values between day 1 and day 85 is not correlated to change in WOMAC pain.

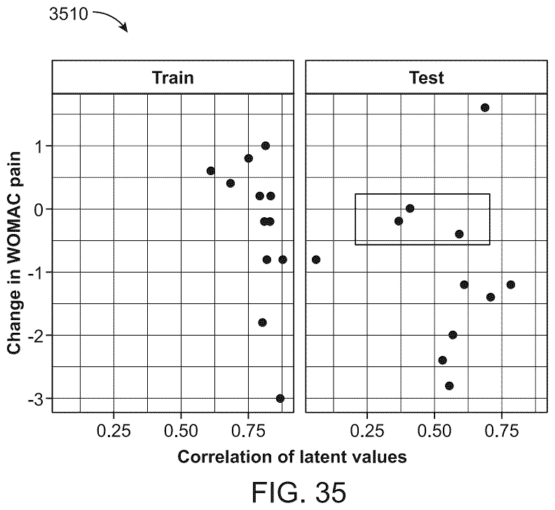

In accordance with the implementations disclosed herein, FIG. 35 includes chart 3510 that shows that correlation of CNN latent values between day 1 and day 85 is not correlated to change in WOMAC pain. The CNN model is trained on day 1 and day 85 for training participants. No significance of spearman correlation is seen in test participants.

In accordance with the implementations disclosed herein, FIG. 36 includes a heat maps 3610 of left and right foot GaitRec data. Control data, knee data, calcaneus data, hip data, and ankle data for a large-scale ground reaction force data set of healthy and impaired gaits are shown. In other words, the heat map 3610 representation may be of vGRF data from GaitRec dataset for all joints with injuries and controls. The data may be z-scored by each column (percent stance phase) across all walks. The heat map may be separated by injury class (control, knee, calcaneus, hip, and ankle), and vGRF from each walk may be unsupervised clustered within each category. The right of the heat map may annotate the joint side with the arthropathy (e.g., left joint, right joint, both joints, or no injury in the control group).

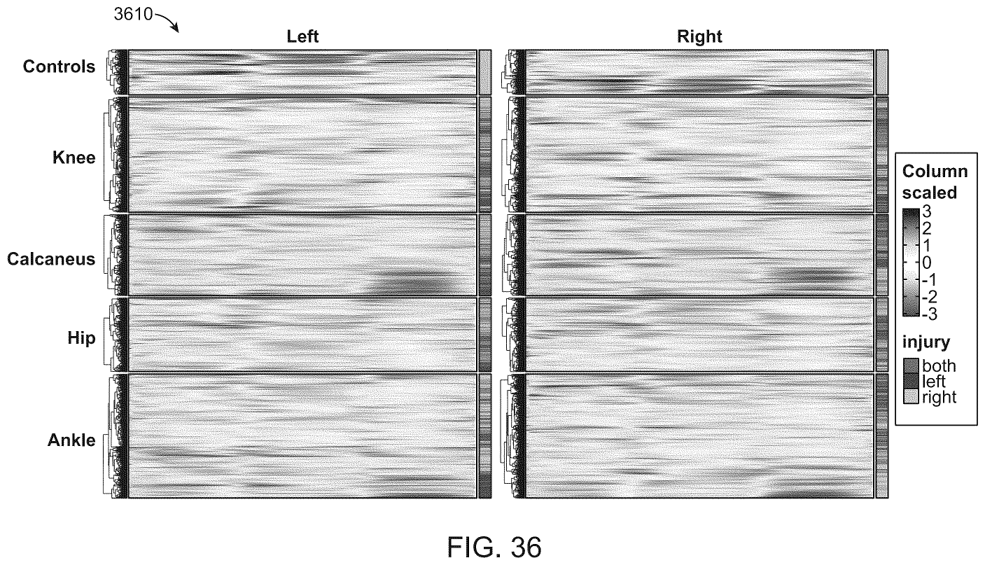

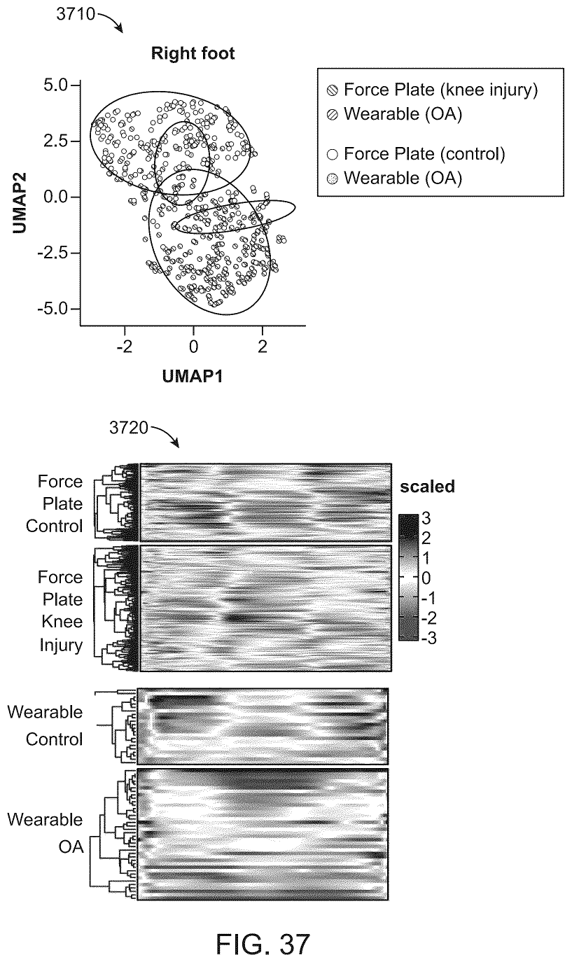

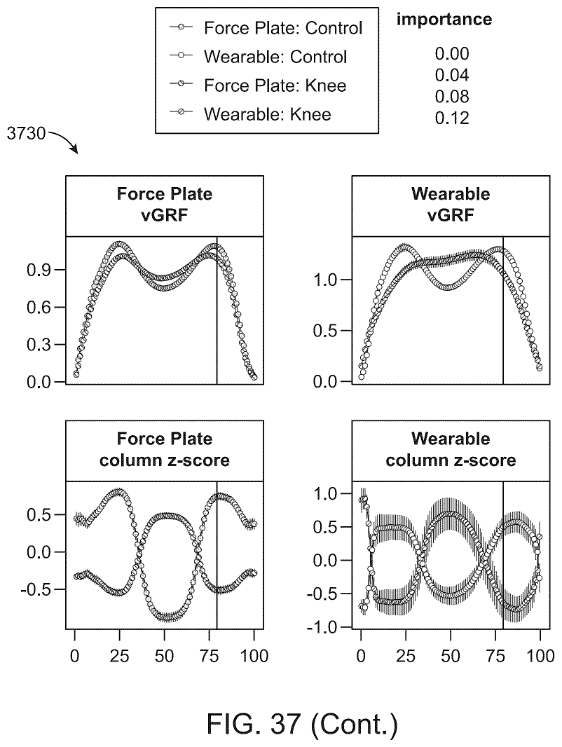

In accordance with the implementations disclosed herein, FIG. 37 shows chart 3710, heat maps 3720, and charts 3730. Chart 3710 shows right foot UMAP data and heat maps 3720 show right foot parameters. Charts 3730 show feature importance learned from a model trained on a force plate dataset.

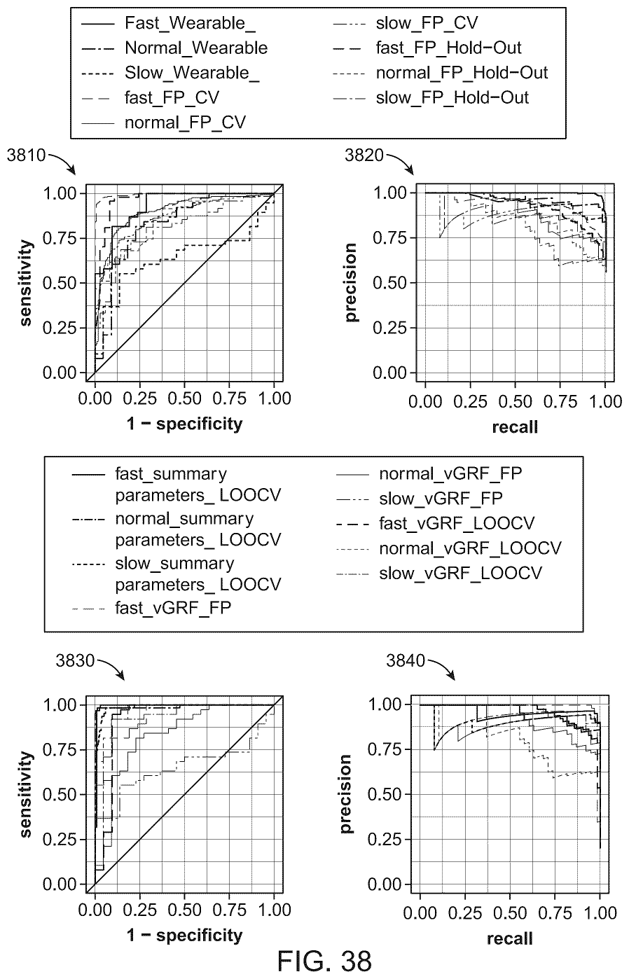

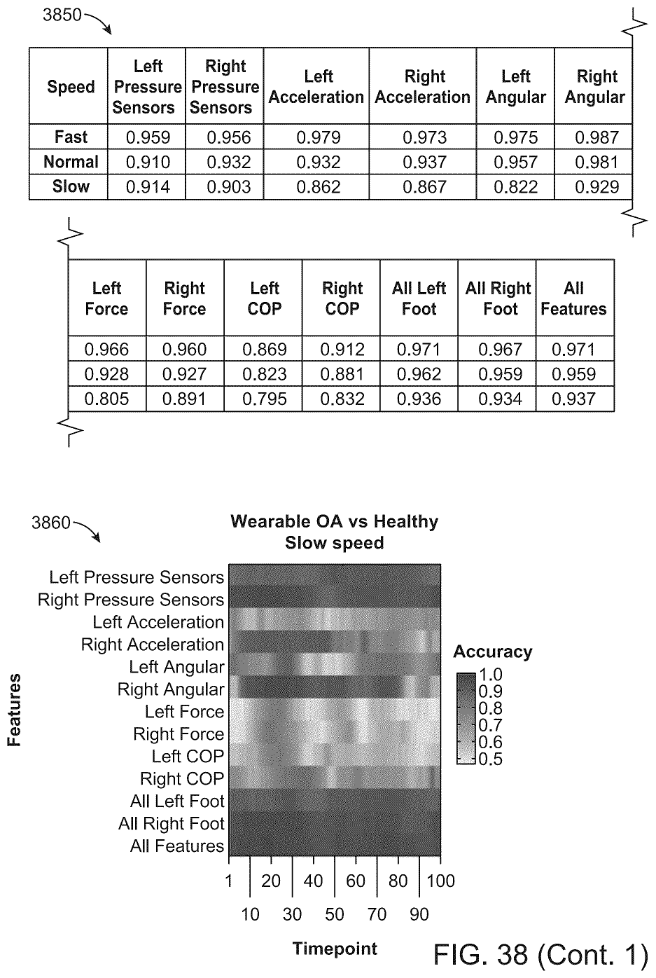

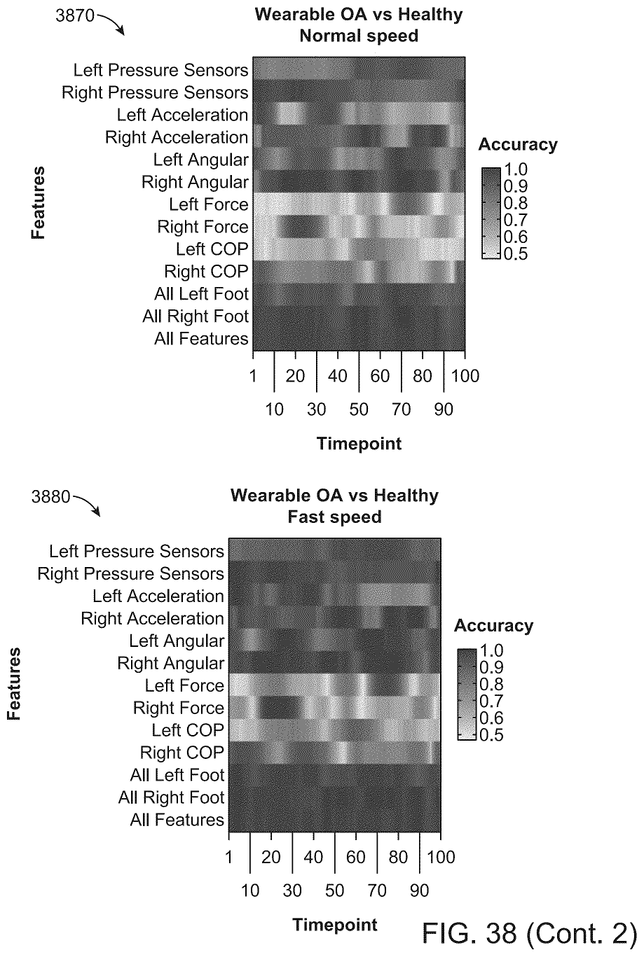

In accordance with the implementations disclosed herein, FIG. 38 shows data and analysis associated with a one or more speeds. Chart 3810 shows sensitivity verses specificity data across different speeds for production and test devices. Chart 3820 shows precision verses recall data corresponding to chart 3810. Chart 3730 shows summary parameters and vGRF sensitivity verses specificity data. Chart 3740 shows precision verses recall data corresponding to chart 3830. Table 3850 shows sensor outputs based on fast, normal, and slow speeds. Heat map 3860 shows wearable OA vs healthy data at slow speeds. Heat map 3870 shows wearable OA vs healthy data at normal speeds. Heat map 3880 shows wearable OA vs healthy data at fast speeds.

In accordance with the implementations disclosed herein, FIG. 39 shows plots 3910 with WOMAC data and plots 3920 with Kellgren and Lawrence (KL) data. Plots 3910 and 3920 show that walking speed is not correlated with disease severity.

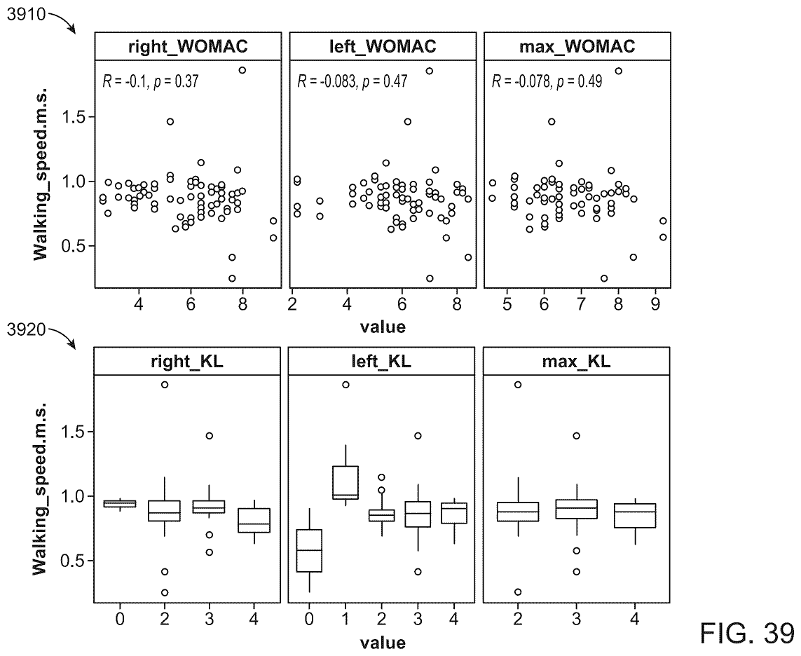

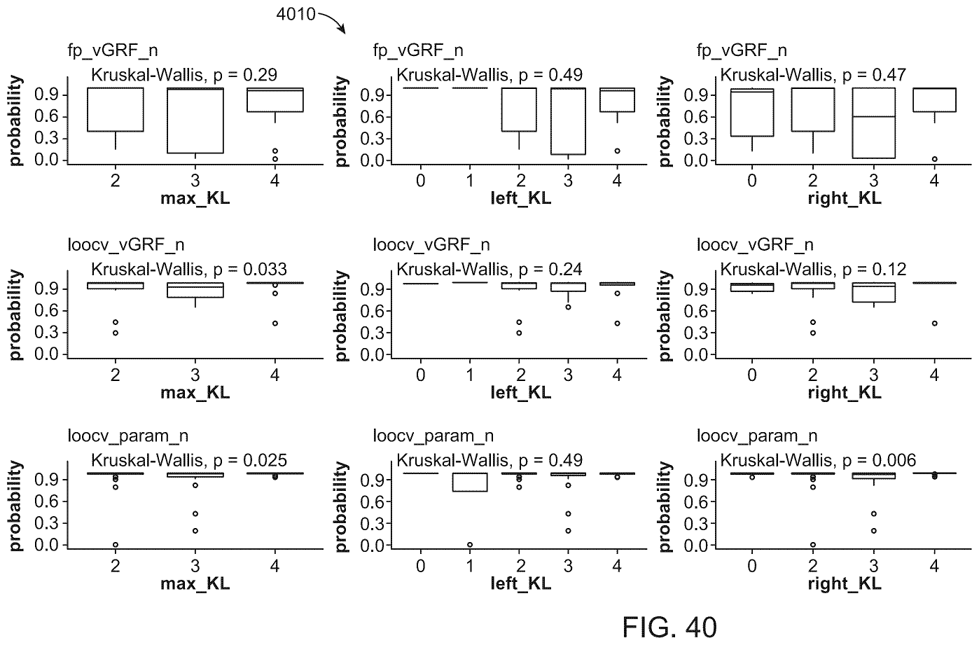

In accordance with the implementations disclosed herein, FIG. 40 includes plots 4010 that that show model predictions at comfortable speeds verses disease severity. Plots 4010 include the data plotted as a probability verses KL and include vGRF data and LOOCV data.

In accordance with the implementations disclosed herein, FIG. 41 includes tables 4110 and 4120 that shows per class binary classification metrics, auROC, auPR, and F1 scores for two datasets.

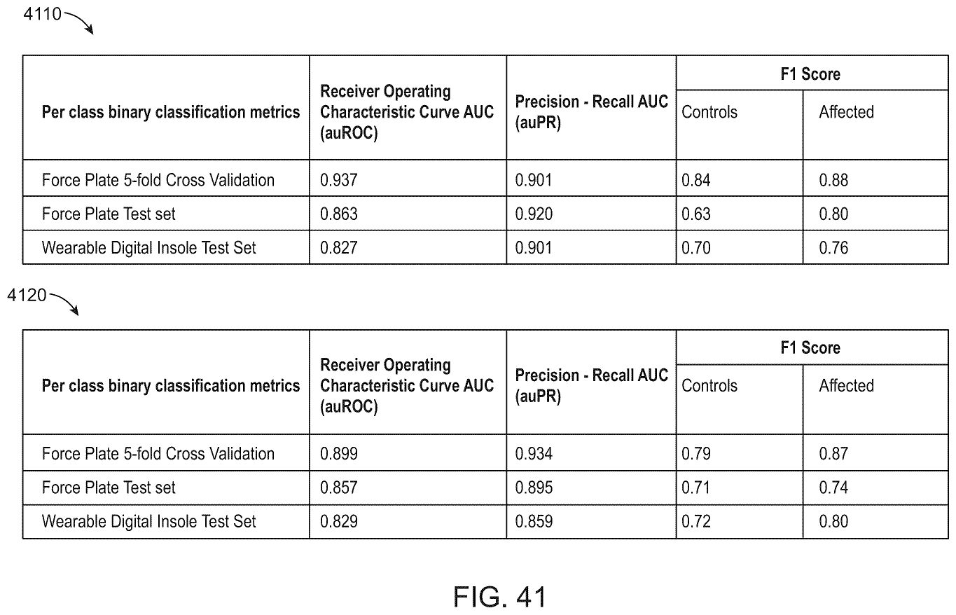

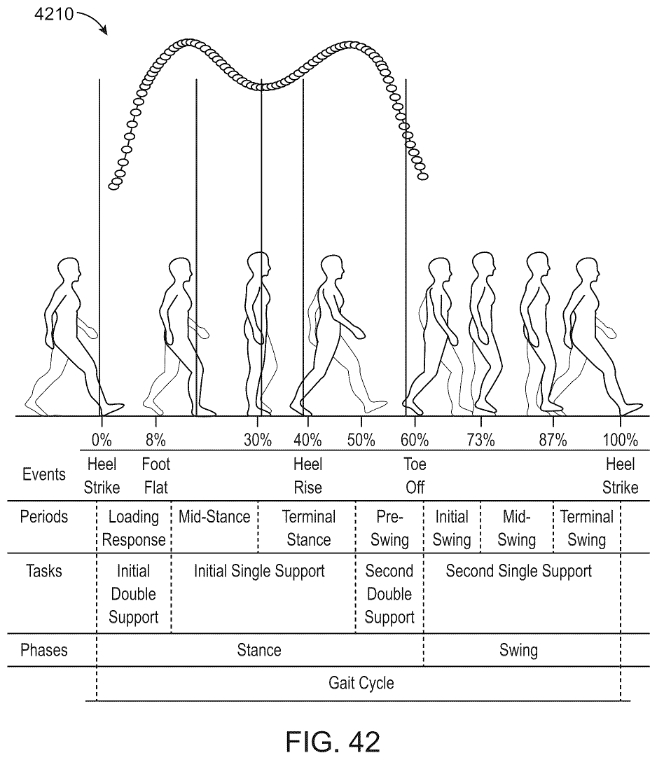

In accordance with the implementations disclosed herein, FIG. 42 depicts a diagram 4210 showing a vGRF plot with gait cycle events, periods, tasks, and phases for a stride. As shown, a single stride may be used to generate a vGRF plot.

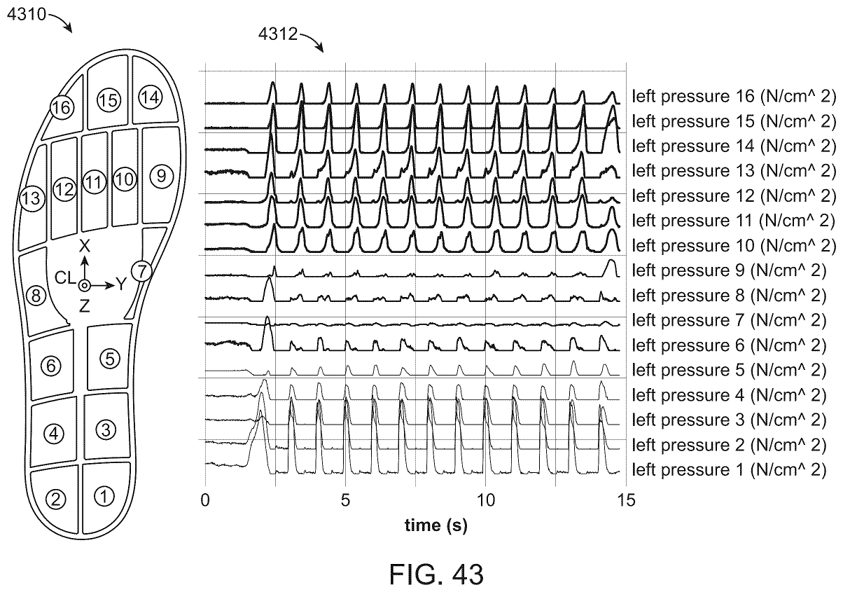

In accordance with the implementations disclosed herein, FIG. 43 shows a wearable insole device 4310 having a plurality of pressure sensors and a motion sensor (e.g., a gyroscope). Chart 4312 shows signals output by each of the sensors (e.g., over the course of a step).

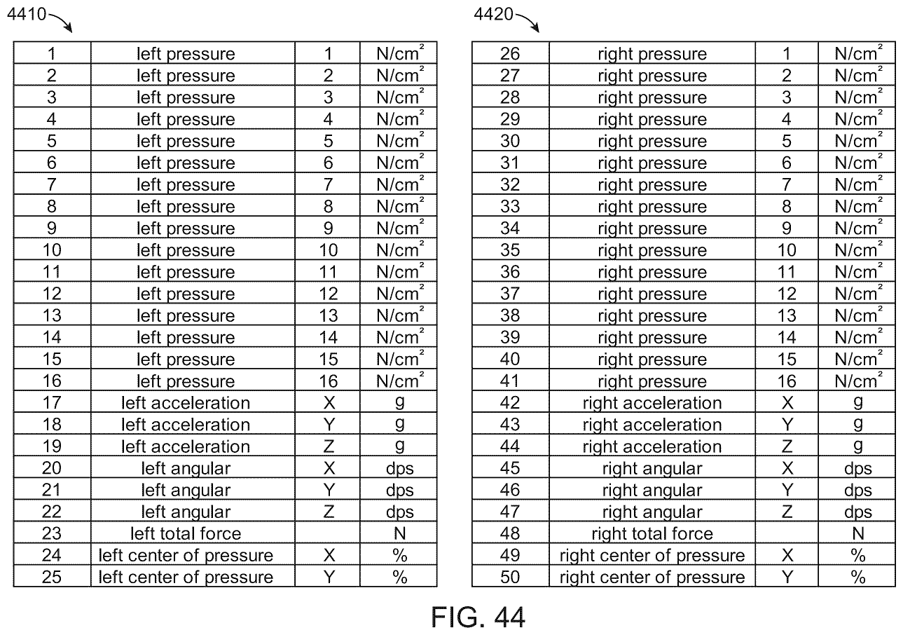

In accordance with the implementations disclosed herein, FIG. 44 shows charts 4410 and 4420 of left pressures, right pressures, acceleration, force, and angular distribution. According to an example, the data shown in charts 4410 and 4420 may be generated using the wearable insole device 4310 of FIG. 43.

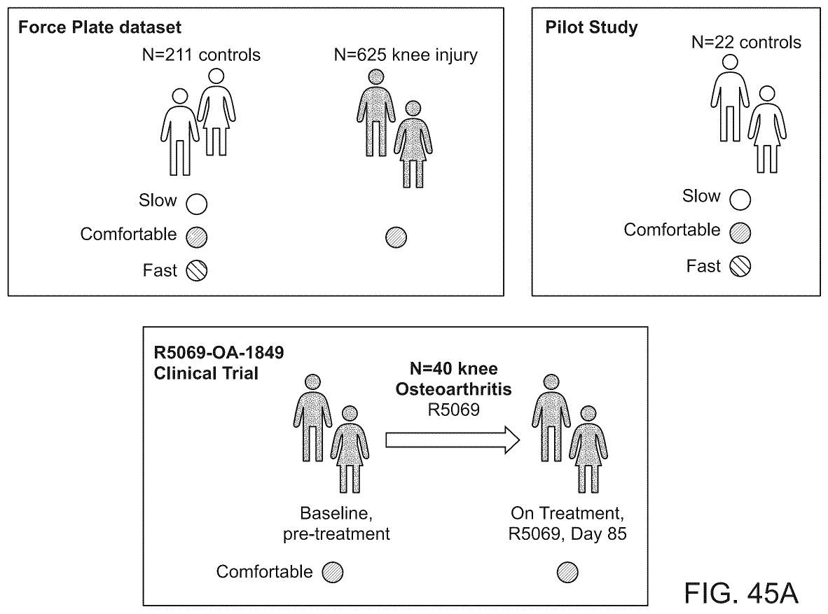

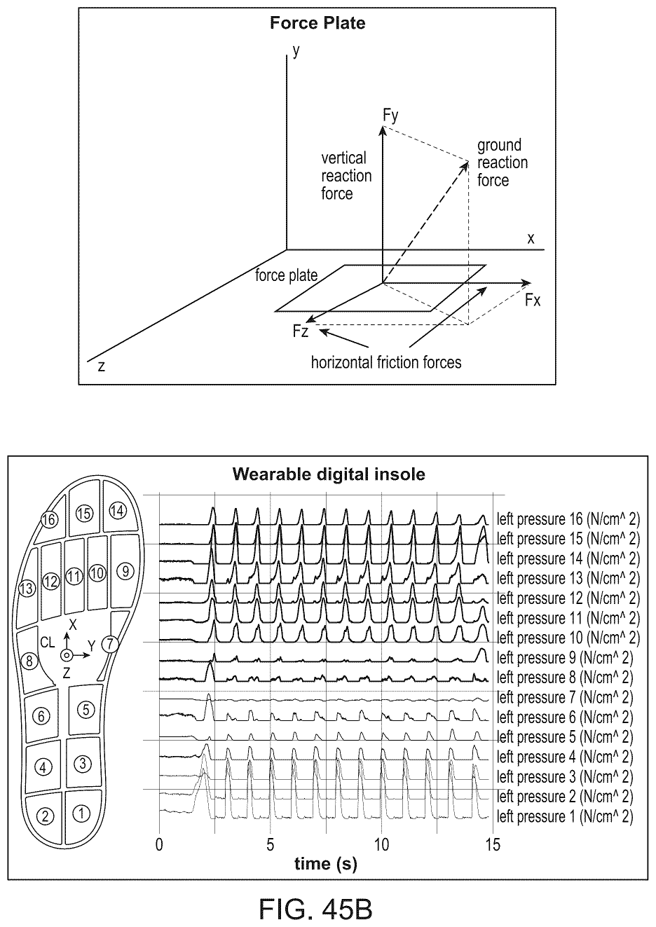

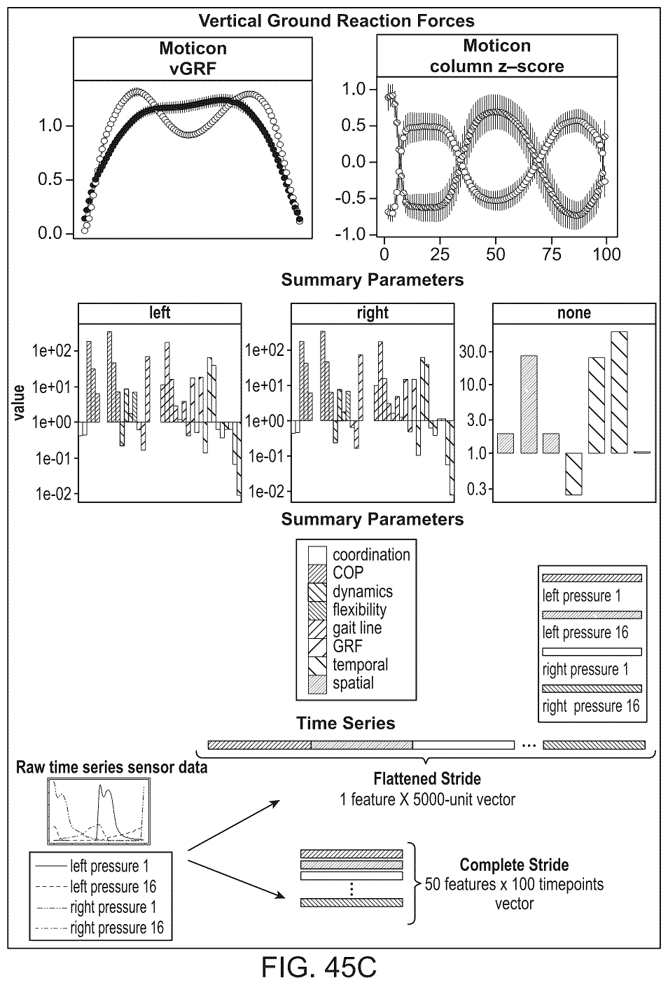

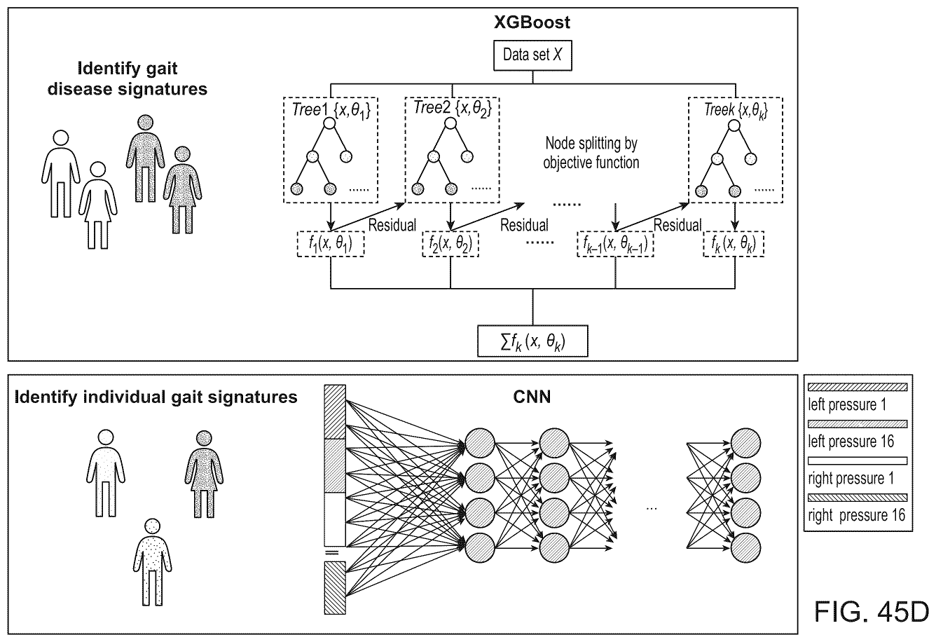

In accordance with the implementations disclosed herein, FIG. 45A shows data from a force plate dataset, a pilot study, and a clinical trial, as discussed herein. FIG. 45B shows force plate data and wearable insole device data. FIG. 45C shows vGRF data, summary parameters, and time series data based on the force plate and wearable insole device data of FIG. 45B. FIG. 45D shows identification of gait disease signatures using an XGBoost model and identification of individual gait signatures using a CNN model, as discussed herein.

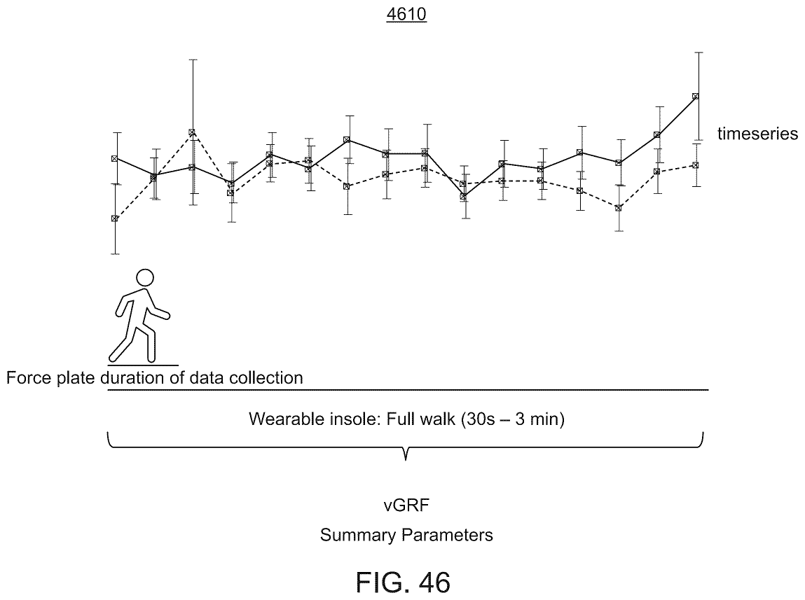

In accordance with the implementations disclosed herein, FIG. 46 shows diagram 4610 of time series data using a force plate and wearable insole device.

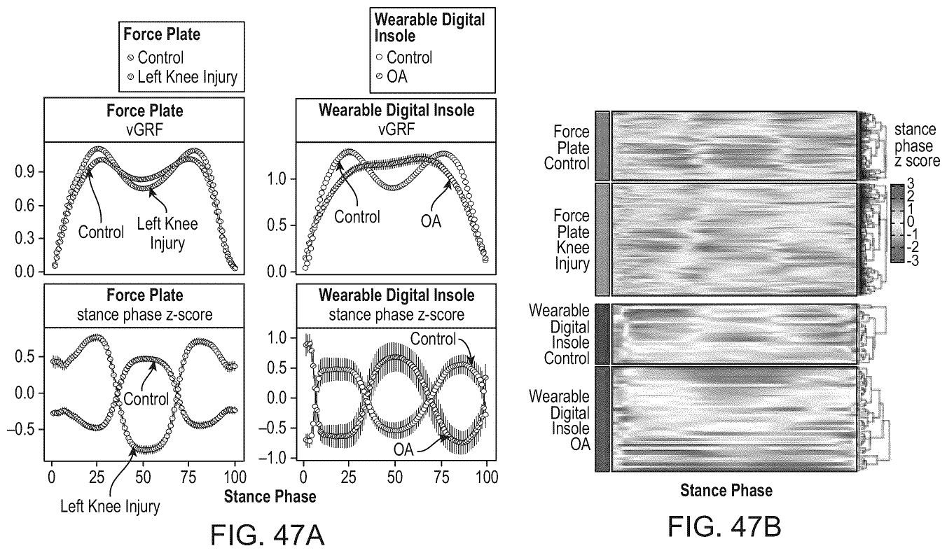

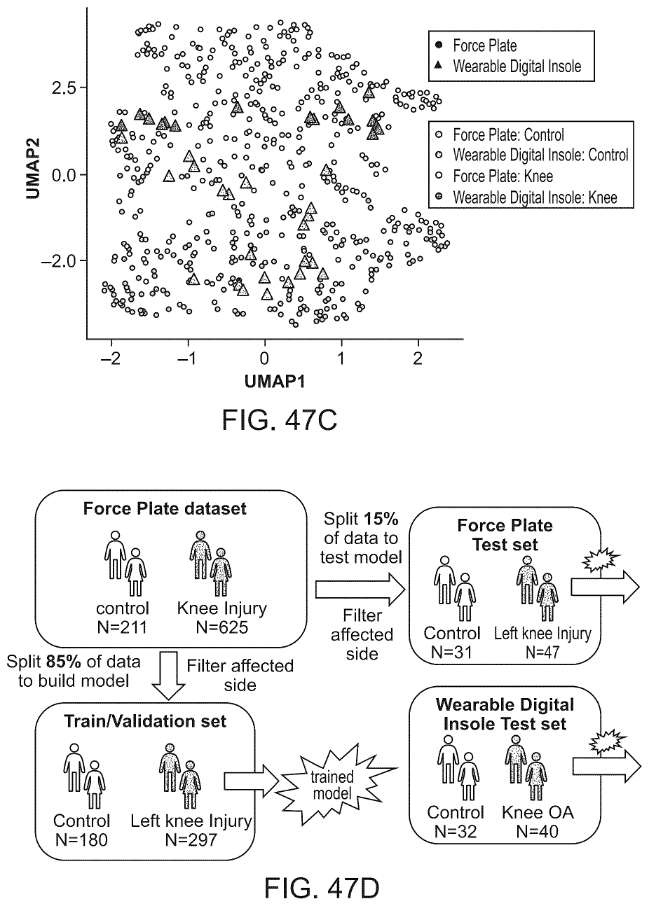

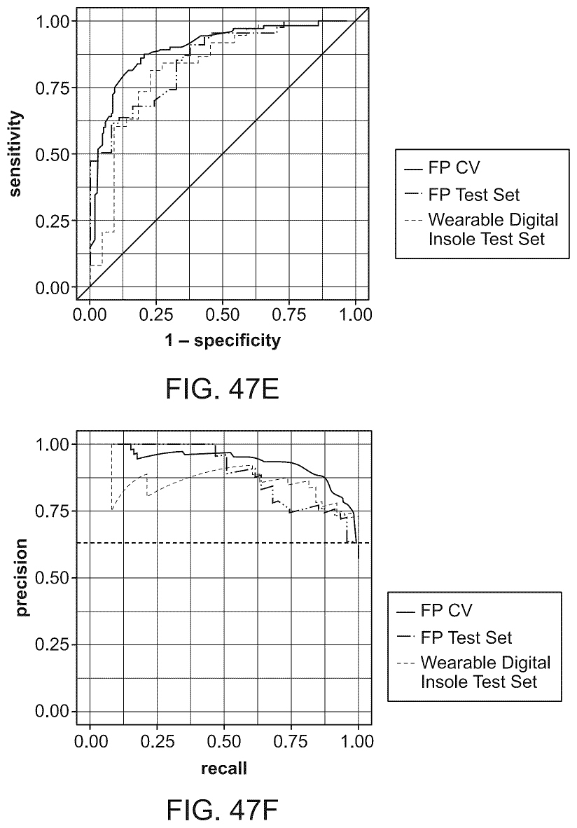

In accordance with the implementations disclosed herein, FIG. 47A-47F show vGRFs measured by force plates and a wearable insole device that distinguish control from knee injury data. FIG. 47A shows vGRF plots and stance phase z-score plots for a force plate and wearable insole device. FIG. 47B shows a heat map generated based on the z-scored data from FIG. 47A. FIG. 47C shows a UMAP plot based on force plate and digital insole device data for both knee injury and control individuals. FIG. 47D shows machine learning models based on a force plate data set, force plate test set, training/validation set, and wearable insole device set, as discussed herein. FIG. 47E shows comparison data plotted based on sensitivity and specificity for force plate and wearable digital insole sets. FIG. 47F shows comparison data plotted based on precision and recall for force plate and wearable digital insole sets.

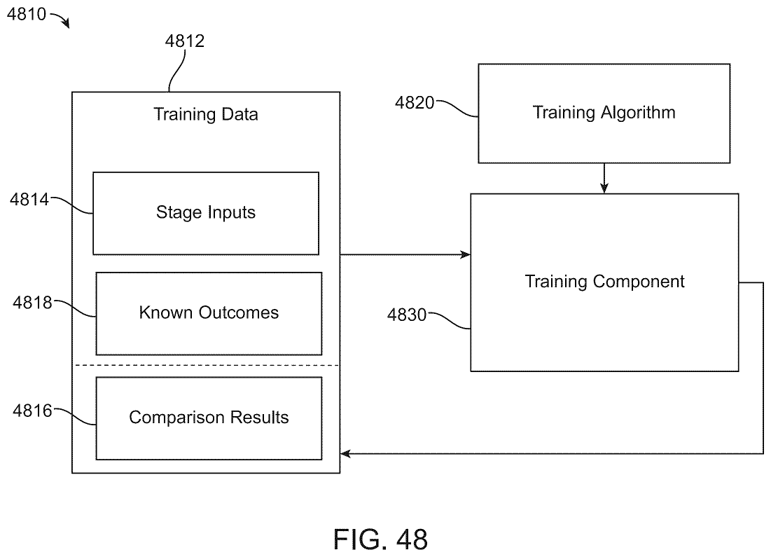

As discussed, one or more implementations disclosed herein include a machine learning model. A machine learning model disclosed herein may be trained using the data flow 4810 of FIG. 48. As shown in FIG. 48, training data 4812 may include one or more of stage inputs 4814 and known outcomes 4818 related to a machine learning model to be trained. The stage inputs 4814 may be from any applicable source including data input or output from a component, step, or module shown in FIGS. 1A-3B. The known outcomes 4818 may be included for machine learning models generated based on supervised or semi-supervised training. An unsupervised machine learning model may not be trained using known outcomes 4818. Known outcomes 4818 may include known or desired outputs for future inputs similar to or in the same category as stage inputs 4814 that do not have corresponding known outputs.

The training data 4812 and a training algorithm 4820 may be provided to a training component 4830 that may apply the training data 4812 to the training algorithm 4820 to generate a machine learning model. According to an implementation, the training component 4830 may be provided comparison results 4816 that compare a previous output of the corresponding machine learning model to apply the previous result to re-train the machine learning model. The comparison results 4816 may be used by the training component 4830 to update the corresponding machine learning model. The training algorithm 4820 may utilize machine learning networks and/or models including, but not limited to a deep learning network such as Deep Neural Networks (DNN), CNN, Fully Convolutional Networks (FCN) and Recurrent Neural Networks (RCN), probabilistic models such as Bayesian Networks and Graphical Models, and/or discriminative models such as Decision Forests and maximum margin methods, or the like.

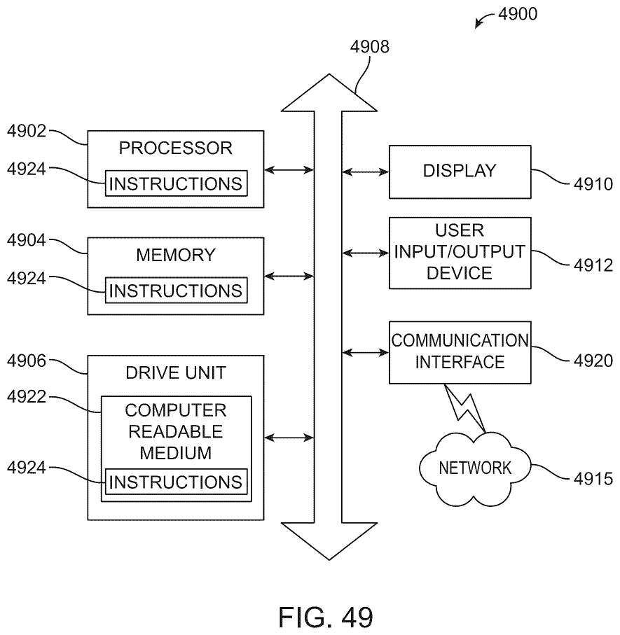

FIG. 49 is a simplified functional block diagram of a computer system 4900 that may be configured as a device for executing the techniques disclosed herein, according to exemplary embodiments of the present disclosure. FIG. 49 is a simplified functional block diagram of a computer system that may generate features, statistics, analysis and/or another system according to exemplary embodiments of the present disclosure. In various embodiments, any of the systems (e.g., computer system 4900) disclosed herein may be an assembly of hardware including, for example, a data communication interface 4920 for packet data communication. The computer system 4900 also may include a central processing unit (“CPU”) 4902, in the form of one or more processors, for executing program instructions 4924. The computer system 4900 may include an internal communication bus 4908, and a storage unit 4906 (such as ROM, HDD, SDD, etc.) that may store data on a computer readable medium 4922, although the computer system 4900 may receive programming and data via network 4915 communications. The computer system 4900 may also have a memory 4904 (such as RAM) storing instructions 4924 for executing techniques presented herein, although the instructions 4924 may be stored temporarily or permanently within other modules of computer system 4900 (e.g., processor 4902 and/or computer readable medium 4922). The computer system 4900 also may include input and output ports 4912 and/or a display 4910 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data, it should be appreciated that the presently disclosed embodiments may be applicable to any environment, such as a desktop or laptop computer, a mobile device, a wearable device, an application, or the like. Also, the presently disclosed embodiments may be applicable to any type of Internet protocol.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

Features enumerated above have been described within the context of particular embodiments. However, as one of ordinary skill in the art would understand, features and aspects of each embodiment may be combined, added to other embodiments, subtracted from an embodiment, etc. in any manner suitable to assist with controlled preparation and/or delivery of a drug.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:

Item 1. A method for validating a test device using a trained machine learning model generated based on a production device, the method comprising:

receiving sensed data from the production device for a control group;

receiving sensed data from the production device for a target group having a target condition;

training a machine learning model to identify a difference between the sensed data for the control group and the sensed data from the target group to generate the trained machine learning model;

providing test sensed data from the test device for a test group comprising a plurality of individuals to the trained machine learning model, the plurality of individuals comprising first individuals having the target condition and second individuals not having the target condition;

receiving a machine learning output from the trained machine learning model, the machine learning output categorizing the plurality of individuals as third individuals having the target condition or fourth individuals not having the target condition;

comparing at least one of the first individuals to the third individuals or the second individuals to the fourth individuals to determine a match value; and

validating the test device if the match value exceeds a match threshold.

Item 2. The method of Item 1, further comprising:

generating control analyzed data based on the sensed data from the production device for the control group;

generating target analyzed data based on the sensed data from the production device for the target group; and

training the machine learning model further based on the control analyzed data and the target analyzed data.

Item 3. The method of Item 2, further comprising:

generating test analyzed data based on the test sensed data from the test device for the plurality of individuals; and

receiving the machine learning output further based on the test analyzed data.

Item 4. The method of Item 1, wherein the test device comprises a plurality of test device sensors and the production device comprises a plurality of production device sensors.

Item 5. The method of Item 4, wherein a density of the plurality of test device sensors is lower than a density of the plurality of production device sensors.

Item 6. The method of Item 4, wherein a sampling frequency of the plurality of test device sensors is lower than a sampling frequency of the plurality of production device sensors.

Item 7. The method of Item 1, wherein the sensed data from test device and the sensed data from the production device each comprise gait sensed data.

Item 8. The method of Item 7, further comprising generating one or more of an average walking speed, a maximum force, a center of pressure, or a bounding box based on the gait sensed data.

Item 9. A method for validating a test device using a machine learning model generated based on a production device, the method comprising:

receiving a machine learning model trained to identify a difference between sensed data from the production device for a control group and sensed data from the production device for a target group, the target group having a target condition;

providing test sensed data from the test device for a test group comprising a plurality of individuals to the trained machine learning model, the plurality of individuals comprising first individuals having the target condition and second individuals not having the target condition;

receiving a machine learning output from the trained machine learning model, the machine learning output categorizing the plurality of individuals as third individuals having the target condition or fourth individuals not having the target condition;

comparing at least one of the first individuals to the third individuals or the second individuals to the fourth individuals to determine a match value; and

validating the test device if the match value exceeds a match threshold.

Item 10. The method of Item 9, further comprising:

receiving the sensed data from the production device for the control group; and

receiving the sensed data from the production device for the target group having a target condition.

Item 11. A method for validating a test device using a trained machine learning model generated using a production device, the method comprising:

receiving sensed data from the production device for a control group;

generating control analyzed data based on the sensed data from the production device for the control group;

receiving sensed data from the production device for a target group having a target condition;

generating target analyzed data based on the sensed data from the production device for the target group;

training a machine learning model to identify a difference between the control analyzed data and the target analyzed data to generate the trained machine learning model;

providing test sensed data from the test device for a test group comprising a plurality of individuals to the trained machine learning model, the plurality of individuals comprising first individuals having the target condition and second individuals as not having the target condition;

receiving a machine learning output from the trained machine learning model, the machine learning output categorizing the plurality of individuals as third individuals having the target condition or fourth individuals not having the target condition;

comparing at least one of the first individuals to the third individuals or the second individuals to the fourth individuals to determine a match value; and

validating the test device if the match value exceeds a match threshold.

Item 12. The method of Item 11, wherein at least one of the generating the control analyzed data or the generating the target analyzed data comprises:

generating a continuous function line based on sensed data; and

generating a stance phase based on the continuous function line.

Item 13. A method for validating a trained machine learning model, the method comprising:

receiving sensed data for a first subset of individuals marked as being in a control group;

receiving sensed data for a first subset of individuals marked as being in a target group having a target condition;

training a machine learning model to identify a difference between the sensed data for the first subset of individuals marked as being in the control group and the sensed data for the first subset of individuals marked as being in the target group, to generate the trained machine learning model;

providing unmarked test sensed data for a test group of individuals to the machine learning model, the test group of individuals comprising a second subset of individuals known to be in the control group and a second subset of individuals known to be in the target group;

receiving a machine learning output from the trained machine learning model, the machine learning output categorizing each of the test group of individuals as being in the control group or being in the target group;

comparing at least one of the test group of individuals categorized as being in the control group to the second subset of individuals known to be in the control group or the test group of individuals categorized as being in the target group to the second subset of individuals known to be in the target group to determine a match value; and

validating the trained machine learning model if the match value exceeds a match threshold.

Item 14. A method for validating a machine learning model, the method comprising:

receiving a machine learning model trained to identify a difference between sensed data for a first subset of individuals marked as being in a control group and sensed data for a first subset of individuals marked as being in a target group;

providing unmarked test sensed data for a test group of individuals to the machine learning model, the test group of individuals comprising a second subset of individuals known to be in the control group and a second subset of individuals known to be in the target group;

receiving a machine learning output from the machine learning model, the machine learning output categorizing each of the test group of individuals as being in the control group or being in the target group;

comparing at least one of the test group of individuals categorized as being in the control group to the second subset of individuals known to be in the control group or the test group of individuals categorized as being in the target group to the second subset of individuals known to be in the target group to determine a match value; and

validating the machine learning model if the match value exceeds a match threshold.

Item 15. The method of Item 14, wherein the machine learning model is trained based on sensed data for a first subset of individuals marked as being in the control group and sensed data for a first subset of individuals marked as being in a target group having a target condition.

Item 16. A method for extracting features using a machine learning model, the method comprising:

receiving sensed data for a first set of individuals;

training a machine learning model to identify features that distinguish each individual in the first set of individuals from each other individual in the first set of individuals, to generate a trained machine learning model; and

extracting the features from the trained machine learning model.

Item 17. The method of Item 16, wherein the sensed data is raw data output by one or more sensors.

Item 18. The method of Item 16, wherein the sensed data for each of the first set of individuals is sensed using a sensing device.

Item 19. The method of Item 18, wherein the sensing device is a wearable insole device.

Item 20. The method of Item 16, wherein the sensed data for each of the first set of individuals is sensed while each individual performs a sensing activity.

Item 21. The method of Item 20, wherein the sensing activity is a movement selected from one or more of a walk, a step, a run, or a jog.

Item 22. The method of Item 16, wherein the features are one of components or differences in the sensed data for the first set of individuals.

Item 23. The method of Item 16, wherein the features are one of components or differences in analyzed signals derived from the sensed data for the first set of individuals.

Item 24. The method of Item 16, wherein extracting the features comprises generating an output based on one or more trained machine learning model components selected from layers, networks, weights, biases, or nodes of the trained machine learning model.

Item 25. The method of Item 16, further comprising validating the features, wherein validating the features comprises:

receiving sensed data for a second set of individuals;

providing the sensed data for the second set of individuals to the trained machine learning model;

receiving a machine learning output categorizing each individual in the second set of data based on the features;

determining a characterization value based on an extent to which each individual in the second set of data is characterized as a unique individual; and

validating the features if the characterization value exceeds a characterization threshold.

Item 26. A method for characterizing unique individuals using a machine learning model, the method comprising:

receiving sensed data for a first set of individuals;

training a machine learning model to identify features that distinguish each individual in the first set of individuals from each other individual in the first set of individuals, to generate a trained machine learning model;

receiving sensed data for a second set of individuals;

providing the sensed data for the second set of individuals to the trained machine learning model; and

receiving a machine learning output characterizing each individual of the second set of individuals as unique individuals based on the features.

Item 27. The method of Item 26, wherein the sensed data is received from a sensing device.

Item 28. The method of Item 27, wherein the sensing device is a wearable insole device.

Item 29. The method of Item 26, wherein the sensed data for each of the first set of individuals is sensed while each individual performs a sensing activity.

Item 30. The method of Item 29, wherein the sensing activity is a movement selected from one or more of a walk, a step, a run, or a jog.

What is claimed is:

1. A method for validating a test device using a trained machine learning model generated based on a production device, the method comprising:
receiving sensed data from the production device for a control group;
receiving sensed data from the production device for a target group having a target condition;
training a machine learning model to identify a difference between the sensed data for the control group and the sensed data from the target group to generate the trained machine learning model;
providing test sensed data from the test device for a test group comprising a plurality of individuals to the trained machine learning model, the plurality of individuals comprising first individuals having the target condition and second individuals not having the target condition;
receiving a machine learning output from the trained machine learning model, the machine learning output categorizing the plurality of individuals as third individuals having the target condition or fourth individuals not having the target condition;
comparing at least one of the first individuals to the third individuals or the second individuals to the fourth individuals to determine a match value; and
validating the test device if the match value exceeds a match threshold.

2. The method of claim 1, further comprising:
generating control analyzed data based on the sensed data from the production device for the control group;
generating target analyzed data based on the sensed data from the production device for the target group; and
training the machine learning model further based on the control analyzed data and the target analyzed data.

3. The method of claim 2, further comprising:
generating test analyzed data based on the test sensed data from the test device for the plurality of individuals; and
receiving the machine learning output further based on the test analyzed data.

4. The method of claim 1, wherein the test device comprises a plurality of test device sensors and the production device comprises a plurality of production device sensors.

5. The method of claim 4, wherein a density of the plurality of test device sensors is lower than a density of the plurality of production device sensors.

6. The method of claim 4, wherein a sampling frequency of the plurality of test device sensors is lower than a sampling frequency of the plurality of production device sensors.

7. The method of claim 1, wherein the sensed data from test device and the sensed data from the production device each comprise gait sensed data.

8. The method of claim 7, further comprising generating one or more of an average walking speed, a maximum force, a center of pressure, or a bounding box based on the gait sensed data.

9. The method of claim 2, wherein at least one of the generating the control analyzed data or the generating the target analyzed data comprises:
generating a continuous function line based on sensed data; and
generating a stance phase based on the continuous function line.

10. A method for validating a machine learning model, the method comprising:
receiving a machine learning model trained to identify a difference between sensed data for a first subset of individuals marked as being in a control group and sensed data for a first subset of individuals marked as being in a target group;
providing unmarked test sensed data for a test group of individuals to the machine learning model, the test group of individuals comprising a second subset of individuals known to be in the control group and a second subset of individuals known to be in the target group;
receiving a machine learning output from the machine learning model, the machine learning output categorizing each of the test group of individuals as being in the control group or being in the target group;
comparing at least one of the test group of individuals categorized as being in the control group to the second subset of individuals known to be in the control group or the test group of individuals categorized as being in the target group to the second subset of individuals known to be in the target group to determine a match value; and
validating the machine learning model if the match value exceeds a match threshold.

11. The method of claim 10, wherein the machine learning model is trained based on sensed data for a first subset of individuals marked as being in the control group and sensed data for a first subset of individuals marked as being in a target group having a target condition.

12. A method for extracting features using a machine learning model, the method comprising:
receiving sensed data for a first set of individuals;
training a machine learning model to identify features that distinguish each individual in the first set of individuals from each other individual in the first set of individuals, to generate a trained machine learning model; and
extracting the features from the trained machine learning model.

13. The method of claim 12, wherein the sensed data is raw data output by one or more sensors.

14. The method of claim 12, wherein the sensed data for each of the first set of individuals is sensed using a sensing device while each individual performs a sensing activity.

15. The method of claim 14, wherein the sensing device is a wearable insole device.

16. The method of claim 14, wherein the sensing activity is a movement selected from one or more of a walk, a step, a run, or a jog.

17. The method of claim 12, wherein the features are one of components or differences in the sensed data for the first set of individuals.

18. The method of claim 12, wherein the features are one of components or differences in analyzed signals derived from the sensed data for the first set of individuals.

19. The method of claim 12, wherein extracting the features comprises generating an output based on one or more trained machine learning model components selected from layers, networks, weights, biases, or nodes of the trained machine learning model.

20. The method of claim 12, further comprising validating the features, wherein validating the features comprises:
receiving sensed data for a second set of individuals;
providing the sensed data for the second set of individuals to the trained machine learning model;
receiving a machine learning output categorizing each individual in the second set of data based on the features;
determining a characterization value based on an extent to which each individual in the second set of data is characterized as a unique individual; and
validating the features if the characterization value exceeds a characterization threshold.
